(19) United States
(12) Patent Application Publication
(10) Pub. No.: US 2026/0085358 A1
(43) Pub. Date: Mar. 26, 2026

(54) RELATIVE AND ABSOLUTE CELL-FREE DNA CONCENTRATIONS FOR CLINICAL UTILITIES

(71) Applicant: Centre for Novostics, Shatin (CN)

(72) Inventors: Yuk-Ming Dennis Lo, Homantin (CN); Kwan Chee Chan, Jordan (CN); Peiyong Jiang, Pak Shek Kok (CN); Wai Kei Lam, Kowloon (CN); Yasine Malki, Tai Po (CN); Guannan Kang, Shatin (CN)

(21) Appl. No.: 19/264,406

(22) Filed: Jul. 9, 2025

Related U.S. Application Data
(60) Provisional application No. 63/669,189, filed on Jul. 9, 2024.

(51) Int. Cl. C12Q 1/6886; C12Q 1/6869; C12Q 1/6881

(52) U.S. Cl. CPC C12Q 1/6886; C12Q 1/6869; C12Q 1/6881; C12Q 2600/166

(57) ABSTRACT

Some techniques of the present disclosure can use size and/or end motifs of cell-free DNA fragments to determine a concentration of cell-free DNA (all or clinically-relevant) in a biological sample of a subject, e.g., as a mass per volume. For example, a measured amount of cell-free DNA fragments at a particular size can be used with one or more calibration amounts determined from one or more calibration samples to determine a concentration of cell-free DNA. As another example, the measured amount can be of cell-free DNA fragments having a set of one or more sequence motifs corresponding to ending sequences of the plurality of cell-free DNA fragments. Certain sequence motif(s) can be selected.

### CROSS-REFERENCES TO RELATED APPLICATIONS

This application claims priority to U.S. Provisional Patent Application No. 63/669,189, entitled “Relative And Absolute Cell-Free DNA Concentrations For Clinical Utilities,” filed on Jul. 9, 2024, the contents of which are hereby incorporated by reference in their entirety for all purposes.

### SEQUENCE LISTING

The instant application contains a Sequence Listing which has been submitted electronically in XML format and is hereby incorporated by reference in its entirety. Said XML copy, created on Oct. 27, 2025, is named 108473-8019US1-1456107.xml and is 2,838 bytes in size.

### BACKGROUND

The demonstration of the presence of circulating cell-free DNA (cfDNA) originating from the fetus in the blood plasma and serum of pregnant women (Lo et al., Lancet 1997; 350:485-487) has completely transformed the practice of prenatal testing through the development of noninvasive prenatal testing (NIPT). NIPT has an advantage in avoiding risks associated with invasive tissue sampling, such as via amniocentesis and chorionic villus sampling (CVS). Thus far, NIPT has been used for fetal RhD blood group genotyping (Finning et al. BMJ 2008; 336:816-818; Lo et al. N Engl J Med 1998; 339:1734-1738), fetal sex determination for sex-linked disorders (Costa et al. N. Engl. J. Med. 2002; 346:1502), chromosomal aneuploidy detection (Chiu et al. Proc Natl Acad Sci USA 2008; 105:20458-20463; Fan et al. Nature 2012; 487:320-324; Chiu et al. BMJ 2011; 342:c7401; Bianchi et al. N. Eng. J. Med. 2014; 460:799-808; Yu et al. Proc. Natl. Acad. Sci. U.S.A 2014; 111:8583-8; Norton et al. N. Engl. J. Med. 2015; 462:1589-1597) and diagnosis of monogenic disorders (Lam et al. Clin. Chem. 2012; 58:1467-75; Lo et al. Sci. Transl. Med. 2010; 2:61ra91-61ra91; Ma et al. Gene 2014; 544:252-258; New et al. J. Clin. Endocrinol. Metab. 2014; 99:E1022-E1030). In particular, using massively parallel sequencing of maternal plasma DNA, NIPT for common chromosomal aneuploidies has been rapidly adopted for clinical service in dozens of countries and is used by millions of pregnant women every year (Allyse et al. Int. J. Womens. Health 2015; 7:113-26; Chandrasekharan et al. Sci Transl Med 2014; 6:231fs15).

In early validation studies (Chiu et al. BMJ2011; 342:c7401; Sparks et al. Am. J Obstet. Gynecol. 2012; 206:319.e1-9), NIPTs were performed on patients at high-risk for aneuploidy, and high positive predictive values (PPVs) have been achieved from 92% to 100%. The relative concentration of fetal DNA in a particular maternal sample, commonly referred to as the fetal DNA fraction, is an important determinant of the accuracy of NIPT (Chiu et al. BMJ 2011; 342:c7401; Jiang et al. Bioinformatics 2012; 28:2883-2890, npj Genomic Med. 2016; 1:16013). The sensitivity of trisomy 21 detection would be significantly decreased with a reduction in the fetal DNA fraction (Chiu et al. BMJ2011; 342:c7401; Canick et al. Prenat. Diagn. 2013; 33:667-674). Hence, false negative results for trisomy detection might occur in pregnancies with low fetal DNA fractions. For example, Canick et al reported that among 212 cases with Down syndrome, there were 4 false negatives, all of which had fetal DNA fractions were between 4% and 7% (Canick et al. Prenat. Diagn. 2013; 33:667-674). Similar dependence on a low tumor DNA fraction can occur for non-invasive cancer detection.

Besides fetal or tumor fraction, the concentration of all circulating cell-free DNA (cfDNA) in a sample (e.g., plasma) can be an important determinant of the robustness of liquid biopsies. Therefore, it would be useful to develop an approach for identifying, assessing, and/or improving the performance of liquid biopsies, e.g., to identify samples with law concentration of all cfDNA, not just that of a fetus, tumor, or transplant tissue.

### BRIEF SUMMARY

Some techniques of the present disclosure can use size and/or end motifs of cell-free DNA fragments to determine a concentration of all cell-free DNA in a biological sample of a subject, e.g., as a mass per volume. For example, a measured amount of cell-free DNA fragments at a particular size can be used with one or more calibration amounts determined from one or more calibration samples to determine a concentration of cell-free DNA. The particular size (e.g., a size range) can be used that (1) the first size has an upper bound less than 231 bp and a lower bound less than 161 bp or (2) the first size has a lower bound greater than 160 bp and an upper bound greater than 230 bp. As another example, the measured amount can be of cell-free DNA fragments having a set of one or more sequence motifs corresponding to ending sequences of the plurality of cell-free DNA fragments. As examples, such a set of one or more sequence motifs can be selected from any sequence motif(s) from Tables 1, 2, 3, 2000, or 2100, or combinations thereof, including equivalent 3-mers of 4-mers listed.

Other techniques of the present disclosure can use size and/or end motifs of cell-free DNA fragments to determine a fractional concentration of clinically-relevant DNA (e.g., tumor, fetal, or transplant) in a biological sample of a subject. For example, a measured amount of cell-free DNA fragments at a particular size can be used with one or more calibration amounts determined from one or more calibration samples to determine a concentration of cell-free DNA. The particular size (e.g., a size range) can be used the first size has a lower bound greater than 160 bp and an upper bound greater than 230 bp. As another example, the measured amount can be of cell-free DNA fragments having a set of one or more sequence motifs corresponding to ending sequences of the plurality of cell-free DNA fragments. As examples, such a set of one or more sequence motifs can be selected from any sequence motifs from Tables 1, 2, 3, 2000, or 2100, or combinations thereof, including equivalent 3-mers of 4-mers listed.

Machine learning models can process a feature vector generated from multiple amounts at different sizes and/or different end motifs.

In some embodiments, a method for measuring a first concentration of all cell-free DNA in a biological sample of a subject is provided. The method includes measuring a first amount of a plurality of cell-free DNA fragments having a first size in the biological sample. The first size can have (1) an upper bound less than 231 bp and a lower bound less than 161 bp or (2) a lower bound greater than 160 bp and an upper bound greater than 230 bp. The method can further include determining the first concentration of all cell-free DNA in the biological sample using the first amount and one or more calibration amounts of cell-free DNA fragments having the first size determined from one or more calibration samples. Each calibration sample may have a known concentration of cell-free DNA.

In some embodiments, a method for measuring a first concentration of all cell-free DNA in a biological sample of a subject is provided. The method can include measuring a first amount of a plurality of cell-free DNA fragments in the biological sample having a set of one or more sequence motifs corresponding to ending sequences of the plurality of cell-free DNA fragments. The method can further include determining the first concentration of all cell-free DNA in the biological sample using the first amount and one or more calibration amounts of cell-free DNA fragments having the set of one or more sequence motifs determined from one or more calibration samples, each having a known concentration of cell-free DNA.

In some embodiments, a method of measuring a fractional concentration of clinically-relevant DNA in a biological sample of a subject is provided. The biological sample can include cell-free DNA. The method can include measuring a first amount of a plurality of cell-free DNA fragments in the biological sample having a set of one or more sequence motifs corresponding to ending sequences of the plurality of cell-free DNA fragments. The set of one or more sequence motifs can be selected from a group consisting of sequence motifs listed in the top 80 sequence motifs listed in any one of Tables 2 and 3. The top sequence motifs can have the lowest p-value or highest absolute Pearson r value as determined for the training set or the testing set. The method can further include determining the fractional concentration of clinically-relevant DNA in the biological sample using the first amount and one or more calibration amounts of cell-free DNA fragments having the set of one or more sequence motifs determined from one or more calibration samples, each having a known concentration of cell-free DNA.

In some embodiments, a method for measuring a fractional concentration of clinically-relevant DNA in a biological sample of a subject is provided. The biological sample can include cell-free DNA. The method can include measuring a first amount of a plurality of cell-free DNA fragments having a first size in the biological sample. The first size may have a lower bound greater than 160 bp and an upper bound greater than 230 bp. The method can further include determining the fractional concentration of clinically-relevant DNA in the biological sample using the first amount and one or more calibration amounts of cell-free DNA fragments having the first size determined from one or more calibration samples, each having a known concentration of cell-free DNA.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows a distribution plot of plasma cell-free DNA concentration among test subjects.

FIG. 2 shows the quantification of the concentration of DNASE1L3 in plasma of subjects with lowest and highest cfDNA concentration.

FIG. 3A shows concentration of DNASE1L3 in plasma between the highest and lowest five subjects according to some embodiments of the present disclosure. FIG. 3B shows concentration of DNASE1L3 in plasma between the highest and lowest 10 subjects according to embodiments of the present disclosure.

FIGS. 4A-4B show immunoblotting for DNASE1L3 plasma protein levels in subjects according to embodiments of the present disclosure. FIG. 4A shows the normalized DNASE1L3 concentrations for each replicate for the highest five subjects. FIG. 4B shows the normalized DNASE1L3 concentrations for each replicate for the lowest five subjects.

FIG. 5A shows the normalized DNASE1L3 concentration for each replicate for the highest 10 subjects according to embodiments of the present disclosure. FIG. 5B shows the norm normalized DNASE1L3 concentration for each replicate for the lowest 10 subjects according to embodiments of the present disclosure.

FIG. 6 shows the correlation between DNASE1L3 concentration measured between two replicates according to embodiments of the present disclosure.

FIGS. 7A-7B show the tissue-origins of cfDNA of subjects with different cfDNA concentrations using the deduced tissue contribution according to embodiments of the present disclosure. FIG. 7A shows the deduced tissue contribution for Liver. FIG. 7B shows the deduced tissue contribution for Neutrophil.

FIGS. 8A-8B show the tissue-origins of cfDNA of subjects with different cfDNA concentrations using the deduced tissue contribution according to embodiments of the present disclosure. FIG. 8A shows the deduced tissue contribution for B-cell. FIG. 8B shows the deduced tissue contribution for T-cell.

FIGS. 9A-9B show the tissue-origins of cfDNA of subjects with different cfDNA concentrations using the deduced tissue contribution according to embodiments of the present disclosure. FIG. 9A shows the deduced tissue contribution for Erythroblast. FIG. 9B shows the deduced tissue contribution for Megakaryocyte.

FIGS. 10A-10B show plots of the size profile of plasma DNA fragments in subjects with the lowest and highest 10% cfDNA concentrations and the median distribution of the cohort, according to embodiments of the present disclosure. FIG. 10A shows the size profile on a linear scale. FIG. 10B shows the size profile on a logarithmic scale.

FIG. 11A shows the mean size profile of plasma DNA fragments plotted on a linear scale. FIG. 11B shows the mean size profile of plasma DNA fragments plotted on logarithmic scale.

FIG. 12A shows a boxplot of the frequency of cfDNA fragments for the 81-90 bp fragment size range. FIG. 12B shows a boxplot of the frequency of cfDNA fragments in the 301-310 bp fragment size range.

FIG. 13 shows the correlation between the frequency of DNA fragments within each 10 bp window bin and cfDNA concentration for the 20-600 bp fragment size range, according to embodiments of the present disclosure. Blue and red labels indicate 10-bp bins with a statistically significant correlation negative or positive correlation to cfDNA concentration, respectively.

FIG. 14A shows the correlation between the frequency of DNA fragments within each 10 bp window bin and cfDNA concentration for the 81-90 bp fragment size range, according to embodiments of the present disclosure. FIG. 14B shows the correlation between the frequency of DNA fragments within each 10 bp window bin and cfDNA concentration for the 301-310 bp fragment size range, according to embodiments of the present disclosure.

FIG. 15 shows the correlation between measured cfDNA concentration and cfDNA concentration predicted by size profile according to embodiments of the present disclosure.

FIG. 16 is a flowchart illustrating a method for a first concentration of all cell-free DNA in a biological sample of a subject, according to embodiments of the present disclosure.

FIG. 17 shows examples for end motifs according to embodiments of the present disclosure.

FIG. 18 is a heatmap analysis with rows indicating a particular 4-mer motif, the first base of the motif highlighted by a specific color in the left-most column (A, C, G and T colored by green, red, yellow, and blue). Each column indicates plasma DNA sample from one subject. The frequency Z-score, calculated for each end motif, is shown by the color scale.

FIG. 19 shows a heatmap analysis showing z-scores of informative end motif frequencies between lowest and highest 10% of subjects across different genomic regions (Alu regions, CpG islands and gene bodies).

FIG. 20 is a table 2000 listing 4-mer end motifs with a significant negative correlation to cfDNA concentration in the studied cohort, according to embodiments of the present disclosure.

FIG. 21 is a table 2100 listing 4-mer end motifs with a significant positive correlation to cfDNA concentration in the studied cohort, according to embodiments of the present disclosure.

FIG. 22 shows a plot between measured cfDNA concentration and cfDNA concentration predicted by end motifs, according to embodiments of the present disclosure.

FIG. 23A shows the correlation between measured cfDNA concentration and cfDNA concentration predicted by end motifs using LASSO regression, according to embodiments of the present disclosure. FIG. 23B shows the correlation between measured cfDNA concentration and cfDNA concentration predicted by end motifs using elastic net regression, according to embodiments of the present disclosure. FIG. 23C shows the correlation between measured cfDNA concentration and cfDNA concentration predicted by end motifs using support vector regression, according to embodiments of the present disclosure.

FIGS. 24A-24B show bar plots of correlation between measured and predicted total plasma cfDNA concentration by an SVR model trained using different numbers of end motifs.

FIG. 24A shows a bar plot of measured and predicted concentration using a ranked order of end motifs. FIG. 24B shows a bar plot of measured and predicted concentration using a random selection of end motifs.

FIG. 25 shows a bar plot of the correlation between measured and predicted total plasma cfDNA concentration using the top 1-5, top 6-10, top 11-15, top 16-20, and top 6-20 ranked end motifs.

FIG. 26 is a flowchart illustrating a method for measuring a first concentration of all cell-free DNA in a biological sample of a subject, according to embodiments of the present disclosure.

FIG. 27 shows a plot of the correlation between measured cfDNA concentration and cfDNA concentration predicted by end motif and size, according to embodiments of the present disclosure.

FIG. 28A shows a plot of the correlation between measured cfDNA concentration and cfDNA concentration predicted by end motifs and size using LASSO regression, according to embodiments of the present disclosure. FIG. 28B shows a plot of the correlation between measured cfDNA concentration and cfDNA concentration predicted by end motifs and size using elastic net regression, according to embodiments of the present disclosure. FIG. 28C shows a plot of the correlation between measured cfDNA concentration and cfDNA concentration predicted by end motifs and size using support vector regression, according to embodiments of the present disclosure.

FIG. 29 shows contributions of different F-profiles for the subjects having the highest and lowest cfDNA concentration for DNA fragments within a size range of 231-600 bp.

FIG. 30 shows box and whisker plots of the contributions from five of the F-profiles for DNA fragments within a size range of 231-600 bp.

FIG. 31 shows box and whisker plots of the contributions from five of the F-profiles for DNA fragments within a size range of 21-160 bp.

FIG. 32 shows box and whisker plots of the contributions from five of the F-profiles for DNA fragments within a size range of 161-230 bp.

FIG. 33 shows a plot of a heatmap analysis, with each row showing the contribution of each F-profile across subjects with different cfDNA concentrations, according to embodiments of the present disclosure.

FIG. 34 shows a plot of a heatmap analysis for visualizing F-profile contributions of plasma DNA between subjects of different cfDNA concentrations for the 21-160 bp size range, according to embodiments of the present disclosure. The F-profile contribution was normalized by Z-score (calculated for each F-profile) towards all 862 subjects.

FIG. 35 shows a plot of a heatmap analysis for visualizing F-profile contributions of plasma DNA between subjects of different cfDNA concentrations for the 161-230 bp size range, according to embodiments of the present disclosure. The F-profile contribution was normalized by Z-score (calculated for each F-profile) towards all 862 subjects.

FIG. 36 is a table showing multiple linear regression analysis of size stratified F-profile contributions to cfDNA concentration in the studied cohort, according to embodiments of the present disclosure.

FIGS. 37A-37B show an application of fragmentomic patterns based deduction of the fractional DNA concentration from fetal cell types.

FIGS. 38A-38B show the accuracy in prediction of the fetal fraction using a size profile between 231-600 bp (FIG. 38A) and using the size profile and end motifs (FIG. 38B).

FIG. 39 shows the accuracy in prediction of fetal fraction using a size profile between 20-600 bp. The data provides evidence that using a size profile to predict fetal fraction may be less accurate than using both size and end motifs.

FIGS. 40A-40B show an application of fragmentomic patterns based deduction of the fractional DNA concentration from tumor cell types.

FIGS. 41A-41B show the accuracy in prediction of the tumor fraction using a size profile between 231-600 bp (FIG. 41A) and using the size profile and end motifs (FIG. 41B).

FIG. 42 shows the correlation between tumor DNA fraction predicted by a size profile between 20-600 bp and copy number aberration (ichorCNA).

FIG. 43A is a table 4300 listing features with the highest coefficient value in the prediction of fetal fraction using LASSO regression. FIG. 43B is a table 4310 listing features with the highest coefficient value in the prediction of tumor fraction using LASSO regression.

FIGS. 44A-44B show bar plots of correlations between the measured and predicted fetal fraction using different numbers of end motifs. FIG. 44A shows a bar plot of correlations for ranked order of end motifs. FIG. 44B shows a bar plot of correlations for random selection of end motifs. FIG. 44C shows a bar plot of a selection of top ranked motifs from DNA of only within 231-600 bp for measuring a fetal fraction.

FIGS. 45A-45B show bar plots of correlation between measured and predicted tumor fraction by an SVR model trained using different numbers of end motifs. FIG. 45A shows a bar plot of correlations for ranked order of end motifs. FIG. 45B shows a bar plot of correlations for random selection of end motifs. FIG. 45C shows a bar plot of a selection of top ranked motifs from DNA of only within 231-600 bp for measuring a tumor fraction.

FIG. 46 is a flowchart illustrating a method for measuring a fractional concentration of clinically-relevant DNA in a biological sample of a subject using end motifs, according to embodiments of the present disclosure.

FIG. 47 is a flowchart illustrating a method for measuring a fractional concentration of clinically-relevant DNA in a biological sample of a subject using fragment size, according to embodiments of the present disclosure.

FIG. 48 illustrates a system according to an embodiment of the present invention.

FIG. 49 shows a block diagram of an example computer system usable with system and methods according to certain embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells.

A “biological sample” refers to any sample that is taken from a subject (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest (e.g., DNA and/or RNA). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, peritoneal dialysate, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed.

Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample, or tissue fraction can refer to the fractional concentration of DNA from one or more particular tissue(s).

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. A region can be defined around a site, e.g., a symmetric or asymmetric region around a site. As examples, a region can include at least +/−50 bases before and after a site (e.g., 101 bases), +/−60 bases, +/−70 bases, +/−80 bases, +/−90 bases, +/−100 bases, +/−150 bases, +/−200 bases, +/−300 bases, +/−400 bases, +/−500 bases, +/−600 bases, +/−700 bases, +/−800 bases, +/−900 bases, and +/−1,000 bases. As other examples a region can be at least 100 bases, 140 bases, 147 bases, or 167 bases long. One or more regions can be analyzed, e.g., to provide a level of a pathology (e.g., cancer) or a fraction of a particular tissue.

Various number of regions, sites, or loci can be analyzed, e.g., 50, 100, 200, 500, 1,000, 5,000, 10,000, 50,000, 100,000, 500,000, one million, or more. Various techniques can determine a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%. A “cutting site” can refer to a location that DNA was cut by a nuclease, thereby resulting in a DNA fragment.

The term “hypomethylation” can refer to a site (hypomethylated site) or set of sites (e.g., a region) that has below a specified threshold for a methylation level, e.g., at or below 50%, 45%, 40%, 35%, 30%, 25%, or 20% for the methylation level. A site in a genome may be considered unmethylated if the methylation level is below a threshold. The term “hypermethylation” can refer to a site (hypermethylated site) or set of sites (e.g., a region) that has above a specified value for a methylation level, e.g., at or above 95%, 90%, 80%, 75%, 70%, 65%, or 60% for the methylation level. A site in a genome may be considered methylated if the methylation level is greater than a threshold. Hypomethylation or hypermethylation can occur for a particular tissue or across a set of tissues.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location. Various alignment tools can be used, such as BLAST, BLASTZ, FASTA, G-PAS, SSEARCH, BOWTIE, AMAP, or SOAP.

A “sequence motif” may refer to a short, recurring pattern of bases in DNA fragments (e.g., cell-free DNA fragments). A sequence motif can occur at an end of a fragment, and thus be part of or include an ending sequence. An “end motif” (also referred to as a “end sequence motif”) can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif. The number of nucleotides (nt) at the fragment ends used for analysis could be, for example, but not limited to, 1 nt, 2 nt, 3 nt, 4 nt, 5 nt, 6 nt, 7 nt, 8 nt, 9 nt, and 10 nt or above. In some embodiments, the fragment end motif could be defined by one or more nucleotides across positions nearby the end of a fragment. The fragment end motif could be defined by one or more nucleotides in a reference genome surrounding the genomic locus to which the end of a fragment is aligned.

A “set of one or more sequence motifs” can correspond to ending sequences of a plurality of cell-free DNA fragments and can be selected in various ways. Various numbers of motifs can be used, e.g., at least 1, 2, 3, 4, 5, 6, 7, 8, 9, 10, 16, 20, 30, 40, 50 60, 64, 70, 80, 90, 100, 150, 200, 250, or 256 end motifs. Certain motif(s) can be selected. The selected motif(s) can be motif(s) with a most importance (e.g., top correlation) for determining a concentration of all cell-free DNA in the biological sample or for determining a fractional concentration of clinically-relevant DNA in the biological sample. For example, in various examples, the top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, or 180 can be selected. As other examples, the selected end motif(s) may not include certain top end motif(s) and only include some lower ranked ones such that the highest ranked motif in the list is less than 1, 2, 3, 4, 5, 6, 7, 8, 9, 10, 11, 12, 13, 14, 15, 16, 17, 18, 19, or 20, e.g., top 6-10, top 11-15, top 16-20, and top 6-20 ranked end motifs.

A “sequence motifpair” or “end motifpair” may refer to a pair of end motifs of a particular DNA fragment. For example, a DNA fragment having an A at the 5′ end of one strand and an A at the 5′ end of the other strand can be defined as having a sequence motif pair of A< >A. As another example, a DNA fragment having an A at the 5′ end of one strand and an T at the 3′ end of the same strand can be defined as having a sequence motif pair of A< >T, which would correspond to an A< >A fragment defined using the 5′ ends of the two strands. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site.

A “end-motif profile” may refer to the relationship of ending sequences (e.g., 1-30 bases) of cell-free DNA fragments (also just referred to as DNA fragments) in a sample. Various relationships can be provided, e.g., an amount of cell-free DNA fragments with a particular ending sequence (end motif), a relative frequency of cell-free DNA fragments with a particular ending sequence compared to one or more other ending sequences. In some instances, the end-motif profiles are determined using other types of parameters, such as size. For example, the end-motif profile can be provided in various ways that illustrate an amount of cell-free DNA fragments having one or more particular ending sequences for a given size (single length or size range). A “reference end-motif profile” or an “F-profile” refers to an end-motif profile that can be generated by applying a factorization algorithm (e.g., non-negative matrix factorization) to relative frequencies of DNA molecules of a given biological sample across a plurality of end motifs (e.g., 256 end motifs).

The terms “size profile” and “size distribution” generally relate to the sizes of DNA fragments in a biological sample. Examples sizes include length (e.g., number of bases/nucleotides) or mass. As examples, a length of a nucleic acid fragment can be determined by sequencing the entire nucleic acid fragment or by aligning paired-end sequence reads to a reference genome. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range.

A “relative frequency” (also referred to just as “frequency”) may refer to a relative value of one amount determined from nucleic acid fragments having a particular characteristic (e.g., an end motif or a size, such as a specified length) to one or more other amounts determined from nucleic acid fragments having a different characteristic. Examples include a ranking or a proportion (e.g., a percentage, fraction (ratio), or concentration). For example, a relative frequency of a particular end motif (e.g., A, CG, TAG, etc.) or end motif pair (e.g., A< >A) can provide a proportion of cell-free DNA fragments that have that end motif or that particular pair end motif pair. Such a proportion can be out of all the end motifs for a set of DNA molecules. As another example, the proportion can be a ratio of an amount for a particular end motif (or pair) relative to an amount of one or more other end motifs. As other examples, the relative frequency can be a ranking of amounts, e.g., raw counts of end motifs. The ranking can be of proportions (ratios) for each end motifs, as another example. Similar relative frequencies can be determined for size.

A “calibration sample” can correspond to a biological sample whose relative concentration or absolute concentration of DNA per volume or mass of the biological sample is known or measured. A calibration sample can also correspond to a fractional concentration of clinically-relevant DNA (e.g., tissue-specific DNA fraction) is known, measured, or determined via a calibration method. For example, for a tumor, a fetus, or transplantation, an allele present in the tissue's (e.g., donor's genome) but absent in the healthy/maternal/recipient's genome can be used as a marker for the tissue corresponding to the clinically-relevant DNA. As another example, a tissue-specific methylation pattern can be used. A calibration sample can have separate measured values (e.g., an amount of fragments with a particular end motif or with a particular size) can be determined to which the known concentration can be assigned.

A “calibration data point” includes a “calibration value” (e.g., an amount of fragments with a particular end motif or with a particular size) and a measured or known concentration of all cell-free DNA or from the clinically-relevant DNA (e.g., DNA of particular tissue type). The calibration value can be determined from relative frequencies (e.g., an aggregate value) as determined for a calibration sample, for which the fractional concentration of the clinically-relevant DNA is known. The calibration data points may be defined in a variety of ways, e.g., as discrete points or as a calibration function (also called a calibration curve or calibration surface).

The calibration function could be derived from additional transformation of the calibration data points. The fractional concentration can be determined in various ways, e.g., using a tissue-specific allele, a tissue-specific methylation value or pattern, and a size distribution of a sample with a known fractional concentration.

The term “classification” as used herein refers to any number(s) or other characters(s) that are associated with a particular property of a sample. For example, a “+” symbol (or the word “positive”) could signify that a sample is classified as having deletions or amplifications.

The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “parameter” as used herein can refer to a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A normalized amount, e.g., a relative frequency, is an example of a parameter.

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, or one million parameters. An ML model can be generated using sample data (e.g., training samples) to make predictions on test data. Various number of training samples can be used, e.g., at least 10, 100, 1,000, 5,000, 10,000, 50,000, 100,000, or at least 200,000 training samples. One example is an unsupervised learning model. Another example type of model is supervised learning that can be used with embodiments of the present disclosure. Example supervised learning models may include different approaches and algorithms including analytical learning, statistical models, artificial neural network (e.g. including convolutional and/or transformer layers), boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ordinal classification, data pre-processing, handling imbalanced datasets, statistical relational learning, or Proaftn (a multicriteria classification algorithm), or an ensemble of any of these types. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to +10%. The term “about” can refer to +5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Cell-free DNA (cfDNA) can occur naturally in the form of short fragments in various types of biological samples, such as in plasma, urine, saliva, cerebrospinal fluid, pleural fluid, amniotic fluid, peritoneal fluid, and ascitic fluid. In contrast to DNA contained in a particular tissue, plasma or other biological samples can carry cfDNA molecules released from dying cells from various tissue. Thus, examination of cfDNA from biological samples can provide minimally invasive access to DNA molecules from the various tissue. This can enable detection and analysis of abnormal or diseased tissue (e.g., organs).

Accordingly, much research effort has been made in obtaining diagnostic information from cell-free DNA (cfDNA) to investigate various physiological and pathological states in the context of pregnancy, oncology, and organ transplantation (Lo et al. 2021). There has also been growing research interest in understanding the biological life cycle of cfDNA molecules in circulation. Several key mechanisms of cfDNA release have been proposed, which include the different modes of cell death, the release of neutrophil extracellular traps, and active cellular secretion (Grabuschnig et al. 2020; Heitzer et al. 2020; Han and Lo 2021). The rapid clearance of cfDNA has been demonstrated by studying the kinetics of fetal cfDNA in pregnant women after delivery (Lo et al. 1999; Yu et al. 2013). Measurement of the cfDNA concentration in plasma at a given time reflects upon an interplay between DNA release from cells and clearance from circulation.

Many pathophysiological conditions are characterized by an altered equilibrium of plasma cfDNA concentration. An elevation of cfDNA concentration has been observed in patients with different cancers (Mattox et al. 2023), systemic lupus erythematosus (Tug et al. 2014) and infectious diseases (Han et al. 2020a; Cheng et al. 2021), when compared to healthy controls. Interestingly, simply performing physical exercise, such as a 40-minute run, could lead to a mean increase of 18-fold in the total cfDNA concentration (Fridlich et al. 2023). The concentration of cfDNA serves as an important parameter in liquid biopsy as it affects the sensitivity of disease diagnosis and the reproducibility of quantitative measurements. An enhanced understanding of the production and clearance of cfDNA may give rise to novel diagnostic approaches with greater sensitivity for early disease detection. Such a postulation has been recently supported by a study, where priming agents were developed to inhibit the clearance processes of cfDNA. This enhanced the detection of circulating tumor DNA by over 10-fold in a mouse model of lung cancer (Martin-Alonso et al. 2024).

To determine cfDNA concentration (e.g., for all cfDNA or just for clinically-relevant DNA), we develop approaches to analyze fragmentomic patterns of cfDNA (e.g., cfDNA fragment sizes, end motifs, or the combination thereof). For example, sequence reads corresponding to ends of one or more cfDNA molecules from a subject can be determined directly from a read or paired reads of a cfDNA molecule or aligned with a reference genome.

One or more nucleotides of end of the read or from the reference genome corresponding to the end of the cfDNA molecules can be an end motif. Additionally or alternatively, a distance between each end of the cfDNA molecules in a single read or from the genome can indicate the size of the cfDNA molecule. Amounts of such cfDNA molecules with a specified size (e.g., a size range) and/or having a certain set of one or more end motifs can be used to determine a cfDNA concentration (e.g., for all cfDNA or just for clinically-relevant DNA). For instance, an amount can be compared to calibration amount, as described in more detail herein.

As part of determining a cfDNA concentration, we develop models for predicting the states of a biological process. For example, a machine learning model can be trained using fragmentomic patterns (e.g., end motif frequency or sizes) of cfDNA molecules from biological samples from subjects of varying concentrations (fractional among cell-free DNA or relative/absolute per volume/mass). In a particular example, a machine learning model can be trained using relative frequencies of particular end motifs of cfDNA molecules from subjects (e.g., healthy or with a condition) having a known concentration. In another example, a machine learning model can be trained using relative frequencies of cfDNA molecules of certain sizes.

Additionally, a machine learning model can be trained using relative frequencies of end motifs for cfDNA molecules and certain sizes, as well as relative frequencies of end motifs for cfDNA molecules of certain sizes. As a result of training, the machine learning models may output a predicted concentration based on receiving input with the relative frequencies of end motifs, the relative frequencies of cfDNA molecules of certain sizes, or both. Thus, the machine learning model can utilize fragmentomic patterns to predict a cfDNA concentration in a biological sample.

In the results below, we show that the total cfDNA concentration was linked to changes in the fragmentomic profiles (i.e., the size profile and end motif distribution) of the cfDNA pool.

Our evidence also shows the involvement of nucleases, such as DNASE1L3 and DFFB, in the modulation of cfDNA concentration. The use of a machine learning model (i.e., the support vector regression model) allowed for the cfDNA concentration to be inferred from fragmentomic features. Models were also be employed in the prediction of fetal and tumoral DNA fractions in pregnant women and patients with HCC, respectively. The capability to accurately estimate the fractional contribution of specific cell types to the plasma DNA pool holds significant clinical relevance, for example, possibly facilitating the advancement in non-invasive prenatal testing, as well as cancer detection and monitoring.

We demonstrate that a selection of end motifs, which we have ranked according to the most well-correlated motifs to either the total or fractional cfDNA concentration, leads to a better prediction of cfDNA concentrations compared to a random selection. We have demonstrated this using the top 5, 10, 20, 40, 80, 160, and 256 end motifs (5′ 4-mer end motifs) in the prediction of the total cfDNA concentration, tumor fraction, and fetal fraction. Accordingly, some embodiments can use targeted approaches using a few informative end motif(s) to provide a cost effective method of assessing cfDNA concentrations. As examples, a set of one or more end motifs can be selected from any sequence motifs from Tables 1, 2, 3, 2000, or 2100, or combinations thereof.

We also demonstrate that measured amounts of certain sizes of cfDNA fragments can be used to determine the total or fractional cfDNA concentration. For example, the certain size can have an upper bound less than 231 bp and a lower bound less than 161 bp. As another example, the certain size can have a lower bound greater than 160 bp and an upper bound greater than 230 bp.

It was surprising that fragmentomic profiles (i.e., the size profile and end motif distribution) could be used to measure a concentration of all DNA and not just clinically-relevant DNA, as other techniques had only considered the different properties of the clinically-relevant DNA, which allowed measuring a concentration of just the clinically-relevant DNA as opposed to all cfDNA in a sample.

It was also surprising that longer DNA (i.e., not short DNA less than 160 bp) could be used to accurately determine a fractional concentration of clinically-relevant DNA. Such longer DNA fragments can have a lower bound greater than 160 bp and an upper bound greater than 230 bp). Other techniques had only considered using short DNA.

It was also surprising that certain end motif(s) could provide an accurate measurement of a fractional concentration of clinically-relevant DNA. Use of a relatively small amount of end motifs (e.g., of varying sequence) can enable low-cost techniques (e.g., PCR-based) to measure such a fractional concentration.

In some embodiments as part of a workflow, an initial part (initial portion) of the sample can be analyzed to determine the total cfDNA concentration or a fractional concentration of clinically-relevant DNA of that initial portion. Clinical samples with low cfDNA concentration can still sequenced and analysed if there is sufficient plasma volume remaining.

For example, the cfDNA concentration can be used along with the remaining volume to estimate the amount of cfDNA present. If the remaining cfDNA is greater than a threshold, then an assay (e.g., sequencing) can be performed on the remaining volume. If the cfDNA concentration is too low and plasma volume is scarce (e.g., estimated remaining cfDNA is below a threshold), embodiments may opt to not perform the assay on the remaining volume as there will not be sufficient data for subsequent analysis.

### I. DISTRIBUTION OF CFDNA CONCENTRATION ACROSS SUBJECTS

The concentration of cfDNA in plasma can be an important determinant of robustness of liquid biopsies and may be governed by an interplay between its release and clearance. The concentration of plasma cfDNA by nucleases may also contribute to inter-individual variations in cfDNA concentrations. In some embodiments, fragmentomic characteristics of cfDNA in individuals with different concentrations of cfDNA can be analyzed based on various techniques.

Examples techniques for measuring cfDNA concentrations are provided below. The skilled person will appreciate that various techniques may be used to measure cfDNA concentrations.

### A. Example Measurement Techniques

In the present study, we have analyzed the distribution of plasma cfDNA concentrations in 862 individuals. These subjects are part of a cohort of individuals who had undergone screening for the early detection of nasopharyngeal carcinoma (NPC) in Hong Kong (Chan et al. 2017; Chan et al. 2023). The NPC screening was performed through the detection of Epstein-Barr virus (EBV) DNA in the circulating cfDNA pool.

Subjects tested as EBV-positive may be subjected to target sequencing of plasma EBV DNA as a reflex test to enhance the specificity of NPC detection (Lam et al. 2018). The subjects were screened for NPC (Chan et al. 2017; Chan et al. 2023), specifically plasma EBV DNA testing by real-time polymerase chain reaction (PCR) for NPC screening. The targeted sequencing data of 862 EBV-positive subjects were used to explore the relationship between the fragmentomic features and cfDNA concentrations. To minimize the potential effects of target capture on the fragmentomic analysis, the ‘off-target’ reads were used to study fragmentomic features of cfDNA.

The exclusion criteria of the screening were individuals with cancer, autoimmune diseases, or those with symptoms of NPC, and the use of systemic glucocorticoids or immunosuppressive therapy. In this study, the cfDNA concentration and sequencing data from the 862 subjects with detectable EBV DNA at baseline screening were used. These subjects did not develop NPC or other types of cancer identified within one year of sample collection.

Blood collection from the screening study was done using Roche Cell-Free DNA Collection Tubes (Cat No. 07832389001). Samples were stored at 4° C. for no longer than six hours before processing. The blood was first centrifuged at 1600×g for 10 min at 4° C. The plasma portion was further subjected to centrifugation at 16,000×g for 10 min at 4° C. to pellet out residual cells and debris. Plasma was stored in aliquots at −80° C. until required for experimental work. Subsequent DNA extractions for all samples were carried out after a single freeze-thaw cycle, with samples frozen during plasma processing and thawed only for DNA extraction. No samples underwent multiple freeze-thaw cycles.

CFDNA was extracted as previously described (Chan et al. 2022; Chan et al. 2023).

Briefly, 2 mL of plasma of each sample was extracted using the MagMAX Cell-Free DNA Isolation Kit in the KingFisher Flex System (ThermoFisher) according to the manufacturer's instructions. The cfDNA concentration of each extracted sample was measured using a Qubit 4 fluorometer instrument (Invitrogen). A fluorescent dye can correlate a mass using samples of a known mass of DNA to an intensity of the signal. Such a calibration using samples manufactured to have a particular concentration can provide a measured of the cfDNA concentration. For example, a function or a table can convert a measured intensity to a specific cfDNA concentration by finding an entry having a matching intensity or inputting the measuring intensity into a calibration function determined from the calibration data points for the known samples manufactured to have a particular cfDNA concentration. Various techniques can be used to measure the concentration besides the use of a fluorescent dye. For example, cfDNA concentration can also be quantified using: 1) Quantitative PCR methods, 2) Spectrophotometer (UV-vis), 3) Capillary electrophoretic methods, 4) digital PCR methods (e.g., droplet digital PCR), and fluorometric techniques.

Clinical screening for Epstein-Barr virus (EBV) DNA for nasopharyngeal cancer (NPC) detection can involve target capture enrichment for viral DNA molecules from the total pool of plasma DNA, as it improves the positive predictive value of NPC detection (Lam et al. 2018). Enrichment in the screening study was performed by a hybridization-based capture with probes that cover the entire EBV genome and selected human autosomal DNA regions. Data from the target capture of all subjects (862 subjects) were retrieved for analysis used in this study. Only off-target DNA fragments were analyzed in this study, to avoid the effects of target capture on fragmentomic analysis. Off-target reads were defined as DNA fragments with no full or partial alignment to the human autosomal target sites.

For the selected subjects with the highest and lowest concentrations of cfDNA in the EBV-positive and EBV-negative cohort, genome-wide sequencing was performed without target capture. Extracted DNA from 2 mL of plasma was performed using TruSeq DNA Nano Library Prep Kit (Illumina), with purification steps done using MinElute Reaction Cleanup (Qiagen) according to the manufacturer's instructions. Adaptor-ligated DNA was amplified using eight cycles of PCR. Quality control of the prepared libraries was done by Qubit and Agilent 4200 TapeStation (Agilent).

As for sequencing and alignment, target-captured DNA libraries were sequenced on NextSeq500 System (Illumina) with 75 bp×2 (150 cycles) paired-end sequencing. Sequenced DNA was aligned to human genome (hg19). Fragmentomic analysis was performed using the paired-end reads uniquely aligned to the human autosomal chromosomes, which was consistent with our previous work (Jiang et al. 2020). Re-analysis of the existing data using GRCh38 (UCSC hg38) human reference genome would not significantly alter the results, as the major difference between the two versions of the human reference genomes is the sequence representation for highly repetitive regions and centromeres. Short sequencing reads obtained from those regions would have multiple alignments and would therefore not be utilized in our downstream analysis.

The P-values for all comparisons are stated in the figures and results section. The Pearson's correlation coefficient, r, was used to assess correlations. Statistical comparisons between two groups were performed using the Wilcoxon rank sum test with two-tailed comparisons. Statistical tests and plots were performed using R Studio and GraphPad Prism 9. A value of P<0.05 was considered statistically significant.

As described above, various techniques can be used to measure the cfDNA concentration, including for validation. For the dPCR option, a ddPCR assay can be performed as previously described (Gai et al. 2023), for a selection of 25 samples from EBV-negative cohort as validation. We have adopted this method to target the valosin-containing protein (VCP), using one set of primer and probe. Briefly, ddPCR reactions can be performed by the QX ONE Droplet Digital PCR System (Bio-Rad). The DNA samples used were eluted with the same volume during DNA extraction. The reaction was prepared in 20 μL, with equal volumes of DNA added per reaction. Components added to the reaction include 2× ddPCR Supermix for probes (Bio-Rad), final concentration of 900 mol/L of each primer and 250 nmol/L of the DNA probe. The ddPCR thermal profile consisted of an initial incubation at 37° C. for 30 minutes, followed by the denaturation step at 95° C. for 10 minutes. It was further followed by 45 cycles of amplification, with each cycle including a denaturation at 94° C. for 30 seconds and annealing at 57° C. for 1 minute. A final incubation at 98° C. for 10 minutes was carried out. Data and calculations were Poisson corrected. To measure the cfDNA concentration, the number of DNA templates for the VCP gene was quantified from cfDNA samples. The primer sequence used for this assay was:

VCP Forward primer:
(SEQ ID NO: 1)
5′ GGGAGGTCTGTGGACCCTATC 3′
VCP Reverse primer:
(SEQ ID NO: 1)
5′ GGGAGGTCTGTGGACCCTATC 3′
Probe sequence:
(SEQ ID NO: 2)
5′ FAM CTTCCCCAACCATCAG MGB 3′

B. Variability of cfDNA Concentration in Subjects

Previous reports of cfDNA concentration in healthy individuals showed considerable variation, ranging from 1-50 ng of DNA per milliliter of plasma (Alborelli et al. 2019; Zhu et al. 2023). We reason that characteristic fragmentomic patterns in the cfDNA pool between subjects with different cfDNA concentrations may hold clues to mechanisms that regulate the levels of cfDNA in plasma. The cohort was used to investigate whether the overall concentration of circulating DNA in plasma is associated with changes in fragmentomic features, which might provide hints towards nuclease-mediated fragmentation, or other factors, that contribute to inter-individual differences in cfDNA concentrations.

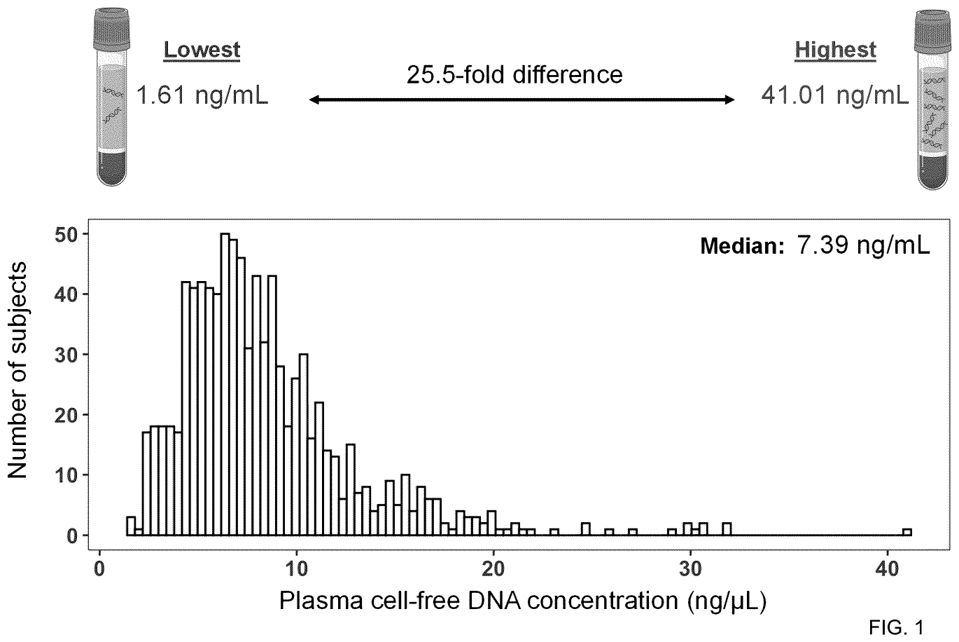

FIG. 1 shows the distribution of cfDNA concentration in test subjects. As shown, the concentration is a mass of cfDNA per volume, but other concentrations of cfDNA can be used, e.g., relative to mass of all material in the sample. As another example, a genomic equivalent per unit volume can be used. A large variation was observed in the cfDNA concentration among subjects, with the concentration ranging from 1.61-41.01 ng/mL, and therefore a 25.5-fold difference between subjects with the highest and lowest plasma cfDNA concentration.

The cfDNA concentrations were shown to have a positively skewed distribution, with a median concentration of 7.39 ng/mL. The skewness of the plasma DNA concentration was 2.043 (P<0.001), and the kurtosis of the distribution was 6.974 (P<0.001). The high positive value of skewness (beyond the range of +1 and −1) indicates a positively skewed distribution of plasma DNA concentrations. The kurtosis value from our cohort represents a leptokurtic distribution (above 3), generally indicating a large proportion of individuals on both extremes of the distribution spectrum. The values of plasma cfDNA concentration deviate from a normal distribution (Shapiro-Wilk test: P<0.001).

### C. Variability and DNASE1L3 Concentration

The activity of DNA nucleases might be at least in part responsible for regulating the concentration of cfDNA in plasma. Previous works have revealed that DNASE1L3 is secreted by macrophages and dendritic cells into plasma circulation (Shiokawa and Tanuma 1998; Sisirak et al. 2016) and plays an important role in the cell-extrinsic fragmentation of cfDNA (Sisirak et al. 2016; Chan et al. 2020). We therefore developed an assay to quantify the concentration of DNASE1L3 in plasma, to determine whether subjects with different cfDNA concentrations were associated with varied levels of DNASE1L3. We focused primarily on the subjects with the highest and lowest cfDNA concentrations, to evaluate if such a difference in DNASE1L3 protein levels was observed.

DNASE1L3 levels in plasma between selected samples were evaluated using the Jess automated Western Blotting system (ProteinSimple). 50 μL aliquots of plasma were diluted 200-fold using 1×phosphate-buffered saline (PBS) and mixed with 2.5×Fluorescent Master Mix containing DTT. The samples were boiled to 95° C. for 5 min. The samples were loaded onto the 12×230 kDa Separation Module containing 25 capillary cartridges with the following settings—Separation voltage: 465 volts, 25 min. ‘Primary Antibody Time’ and ‘Secondary Antibody Time’ as 30 min. ‘Detection Profile—Chemi’. DNASE1L3 immuno-detected using polyclonal anti-DNASE1L3 antibodies (Thermo Fisher PA5-107113) and Secondary Anti-Rabbit antibodies (Anti-Rabbit Detection Module, DM001 Protein Simple). Chemiluminescent signals were quantified by band intensity area using Compass for SW software. Samples were run in two replicates, with the correlation between two replicates.

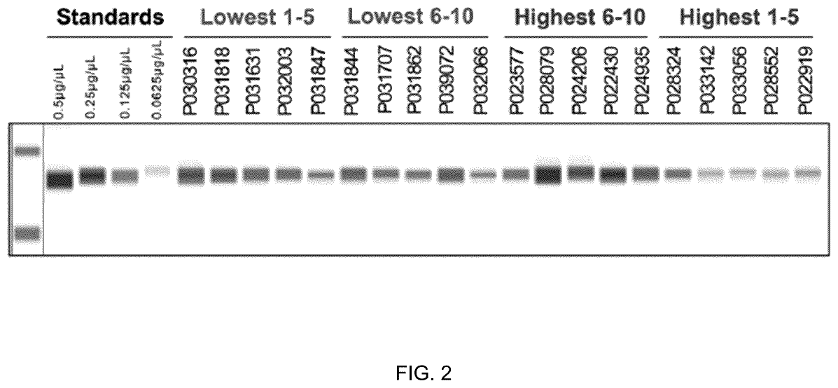

FIG. 2 shows the quantification of the concentration of DNASE1L3 in plasma of subjects with lowest and highest cfDNA concentration. Automated Western blotting was performed for each individual's plasma sample using anti-DNASE1L3 antibodies. DNASE1L3 band intensity area was quantified for each subject. FIG. 2 shows a representative immunoblot of the highest and lowest 10 subjects. Between the lowest and highest 5 subjects for cfDNA concentration, there is a difference with the lowest 5 subjects having a stronger DNASE1L3 concentration than the highest 5 subjects.

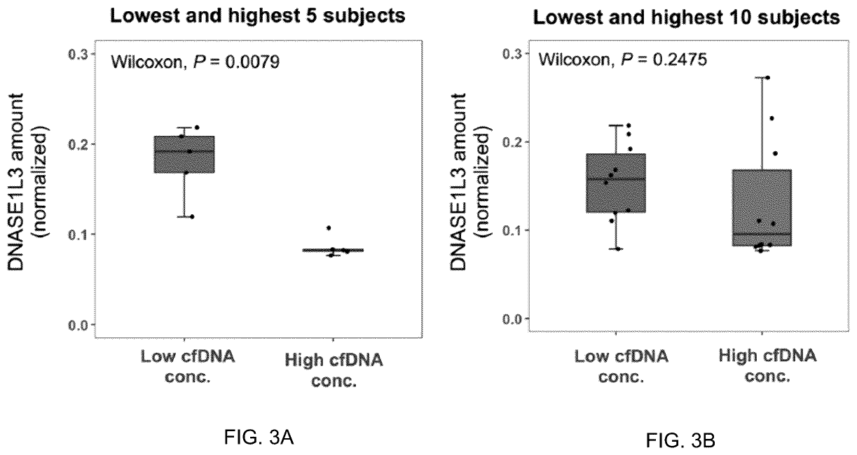

FIG. 3A shows concentration of DNASE1L3 in plasma between the highest and lowest five subjects. FIG. 3B shows concentration of DNASE1L3 in plasma between the highest and lowest 10 subjects. Quantification of band intensity area was normalized to standards with different amounts of plasma proteins. FIG. 3A shows the difference in the highest and lowest 5 subjects as was seen in FIG. 2. Thus, the cfDNA concentration may be caused by the presence of DNASE1L3.

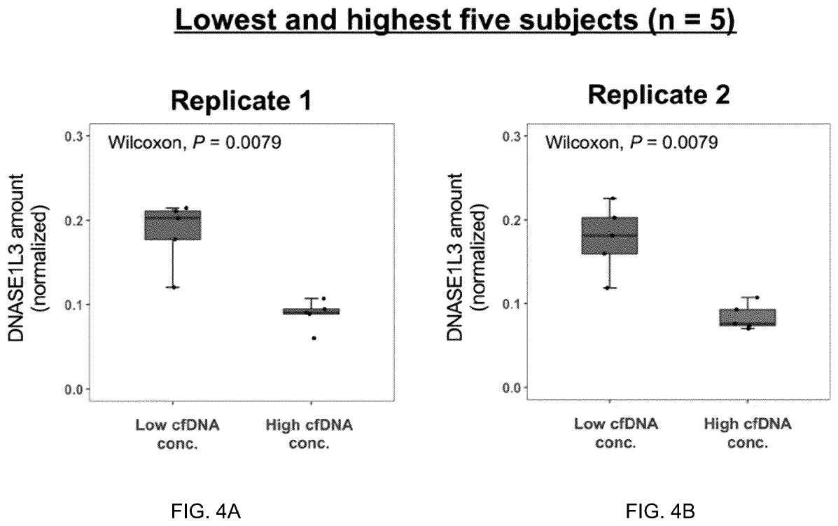

FIGS. 4A-4B show immunoblotting for DNASE1L3 plasma protein levels in subjects of both cohorts. Immunoblotting was performed in replicates. FIG. 4A shows the normalized DNASE1L3 concentrations for each replicate for the highest five subjects. FIG. 4B shows the normalized DNASE1L3 concentrations for each replicate for the lowest five subjects. Again FIGS. 4A-4B show the difference in the highest and lowest 5 subjects as was seen in FIG. 2 and FIG. 3A. Thus, the cfDNA concentration may be caused by the presence of DNASE1L3.

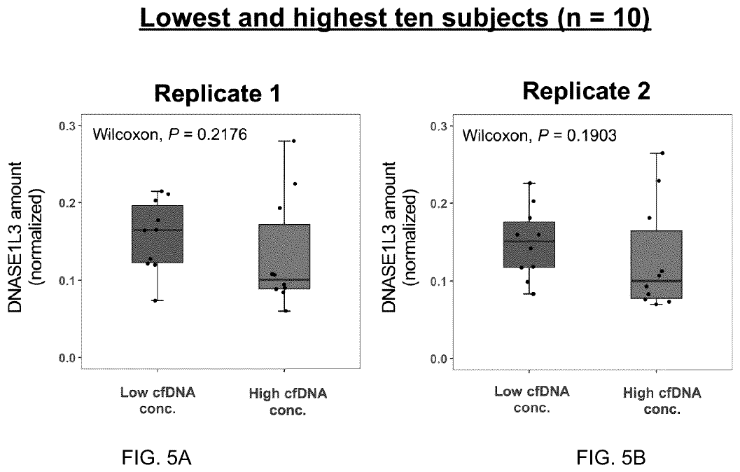

FIG. 5A shows the normalized DNASE1L3 concentration for each replicate for the highest 10 subjects. FIG. 5B shows the norm normalized DNASE1L3 concentration for each replicate for the lowest 10 subjects. The difference in DNASE1L3 amount for the low and high cfDNA subjects is still present but less so, as compared to just the highest and lowest 5 subjects.

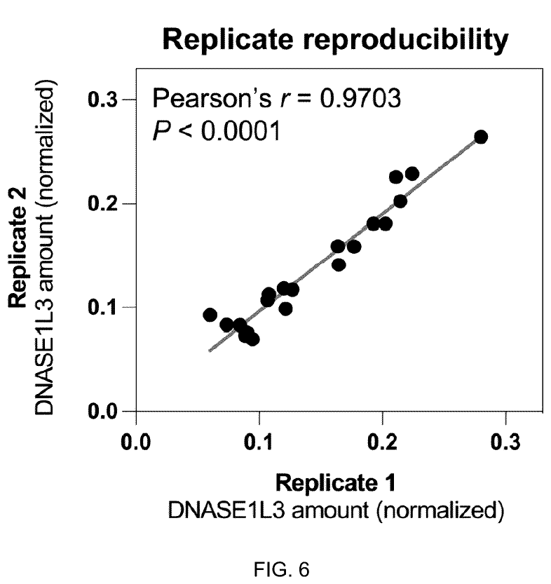

FIG. 6 shows the correlation between DNASE1L3 concentration measured between two replicates. This shows that the replication measurements are consistent.

The highest five subjects showed a significantly decreased level of DNASE1L3 protein in plasma than the lowest five subjects (52.5% reduction in DNASE1L3 levels; P=0.0079, Wilcoxon test, FIGS. 2-6). However, the significance of this trend was diminished when extending to the highest and lowest ten subjects (14.5% reduction in DNASE1L3 level; P=0.2475, Wilcoxon test, FIGS. 2-6). This analysis suggested that the levels of DNASE1L3 in plasma might partially account for the variation in cfDNA concentrations in different individuals.

This current work revealed the link between DNASE1L3-related fragmentation signatures and cfDNA concentration. Subjects with high cfDNA concentrations exhibited fragmentomic features that resembled DNASE1L3-deficient mouse model and human subjects, such as the enhanced di- and tri-nucleosomal patterns in size profile, and decreased C-end motifs (Serpas et al. 2019; Chan et al. 2020). The contribution of DNASE1L3 cleavage profile (Profile I) showed a decreasing frequency with cfDNA concentration, and was most noticeable in the 231-600 bp size range. It is possible that an attenuated DNASE1L3 activity might hinder the degradation of longer cfDNA molecules into shorter molecules.

The reduced activity of DNASE1L3 was at least partially evidenced by the decreased protein levels of DNASE1L3 in plasma from subjects with the highest cfDNA concentrations (comparing the five highest- and lowest-ranked subjects). However, this trend was diminished when expanding further to the highest and lowest 10 subjects. Only individuals with extremely elevated cfDNA concentrations (i.e., approximately 30 ng/mL or above), might be associated with a deficiency in DNASE1L3 concentrations in plasma. Such individuals represented the top 0.58% (top 5 of 862 individuals) in terms of plasma cfDNA concentrations from our cohort. It is also worth noting that the contribution of DFFB also shows a similar gradation pattern with cfDNA concentration, with a decreased contribution in subjects with higher cfDNA concentration. DFFB plays a major role in DNA fragmentation during apoptosis, and our previous work has demonstrated that newly released DNA exhibited strong A-end preference which was associated with DFFB activity (Han et al. 2020b).

An example use of properties of nucleases to determine cfDNA concentration is described in section IV.B.

### D. Tissue-of-Origin Analysis

As we observed that the activity of nucleases was linked to cfDNA concentration, we questioned whether the variation in cfDNA concentration may be associated to the release of cfDNA from a particular cellular source. We employed the fragmentomics-based methylation analysis (FRAGMA) to deduce the methylation status of cfDNA (Zhou et al. 2022). We selected cell-type specific hyper- and hypomethylated CpG sites to six cell types—liver, neutrophils, B-cells, T-cells, erythroblasts, and megakaryocytes, as they represented major cellular sources that contributed to the cfDNA pool (Loyfer et al. 2023). The deduced tissue contribution was assessed based on methylation status at these CpG sites and was correlated to the cfDNA concentration.

To deduce the tissue contributions, a FRAGMA-based tissue deconvolution analysis was used based on the deduction of the methylation status of cytosine-phosphate-guanine (CpG) sites, by the preferential cleavage of methylation sites compared to unmethylated sites, using the CGN/NCG motif ratios as previously described (Zhou et al. 2022). Hypermethylated and Hypomethylated CpG sites are defined as CpG sites with a methylation index of above 70% and below 30%, respectively. Unique hypermethylated CpG sites for each cell type (Liver, neutrophils, B-cells, T-cells, erythroblasts, and megakaryocytes) were identified by bisulfite sequencing tissue references for each cell type. The percentage contribution of each tissue was deduced by the difference in CGN/NCG motif ratio between hyper- and hypomethylated CpG sites, normalized by the CGN/NCG motif ratio from the reference tissue, using the following equation:

$$Normalized C G N / N C G Motif Ratio = \frac{Ratio M - Ratio U}{Reference M - Reference U}$$

Where ‘Ratio’ denotes the raw CGN/NCG motif ratio, ‘M’ and ‘U’ representing methylation and unmethylated sites, respectively.

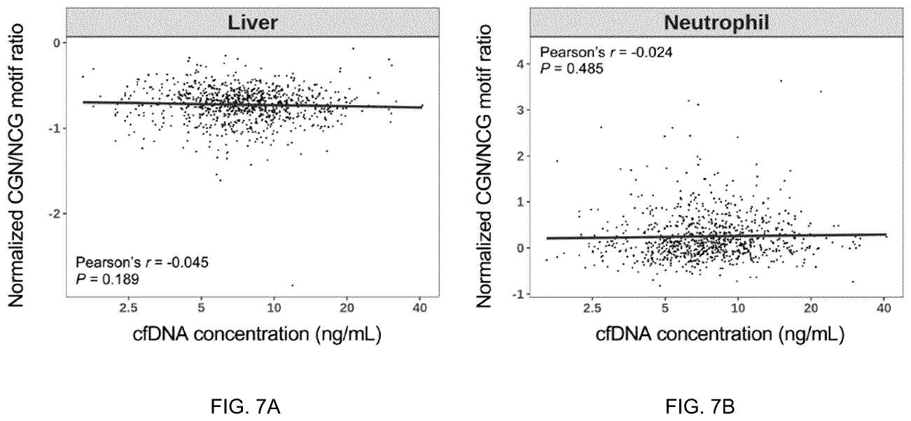

FIGS. 7A-7B show the tissue-origins of cfDNA of subjects with different cfDNA concentrations using the deduced tissue contribution according to embodiments of the present disclosure. Tissue-origins of cfDNA of subjects with different cfDNA concentrations deduced by Fragmentomics-Based Methylation Analysis (FRAGMA). The deduced tissue contribution, expressed as the normalized CGN/NCG motif ratio from selected tissue/cell-type specific CpG sites, was correlated with cfDNA concentration (n=862 healthy individuals from the EVB positive cohort). FIG. 7A shows the deduced tissue contribution for Liver. FIG. 7B shows the deduced tissue contribution for Neutrophil.

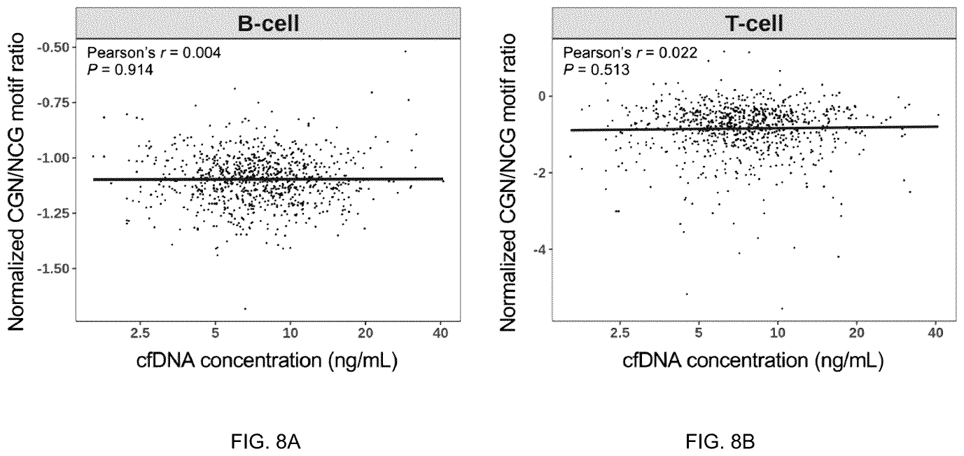

FIGS. 8A-8B show the tissue-origins of cfDNA of subjects with different cfDNA concentrations using the deduced tissue contribution according to embodiments of the present disclosure. FIG. 8A shows the deduced tissue contribution for B-cell. FIG. 8B shows the deduced tissue contribution for T-cell.

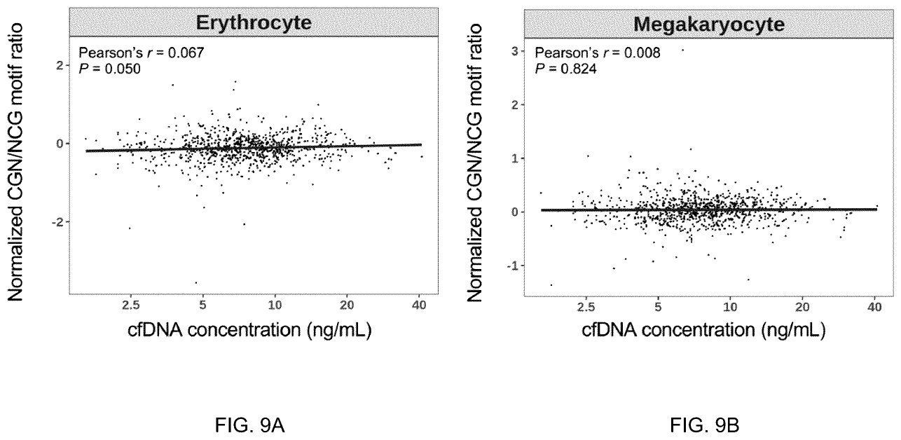

FIGS. 9A-9B show the tissue-origins of cfDNA of subjects with different cfDNA concentrations using the deduced tissue contribution according to embodiments of the present disclosure. FIG. 9A shows the deduced tissue contribution for Erythroblast. FIG. 9B shows the deduced tissue contribution for Megakaryocyte.

The results showed that the deduced contribution of each cell type was not significantly correlated to cfDNA concentration, with a weak correlation coefficient (Pearson r<0.1 for all cell types). Hence, the production of cfDNA from various cell types might not be an important factor contributing to the variation in cfDNA. Given the lack of correlation to overall cfDNA concentration, the results in section III for use of end motifs to determined cfDNA concentration for all cfDNA molecules are even more surprising

### II. SIZE ANALYSIS

A fragment size can relate to a number of base pairs (also referred to as bases for length of a single strand) that make up a cell-free DNA (cfDNA) fragment. CfDNA fragments can be relatively short. For example, cfDNA fragments may predominantly around 160-180 base pairs long. The size distribution of cfDNA fragments can provide valuable insights into their cellular origins and the physiological or pathological processes occurring within a subject. Techniques such as next-generation sequencing (e.g., of entire fragment or alignment of ends to a reference genome), electrophoresis, or other bioanalytical platforms can be used to determine the fragment sizes.

### A. Size of Fragments for Low and High Concentration Samples

In some embodiments, sizes of cfDNA fragments can be analyzed and used to determine cfDNA concentration within a subject. The sizes were measured by performing paired-end sequencing of the DNA fragments to get paired-end reads, which were then aligned to the reference genome. The coordinates of the aligned paired-end reads provide a length (an example of size) of the DNA fragment. In other examples, an entire DNA fragment can be sequenced, thereby providing the length of the DNA fragment.

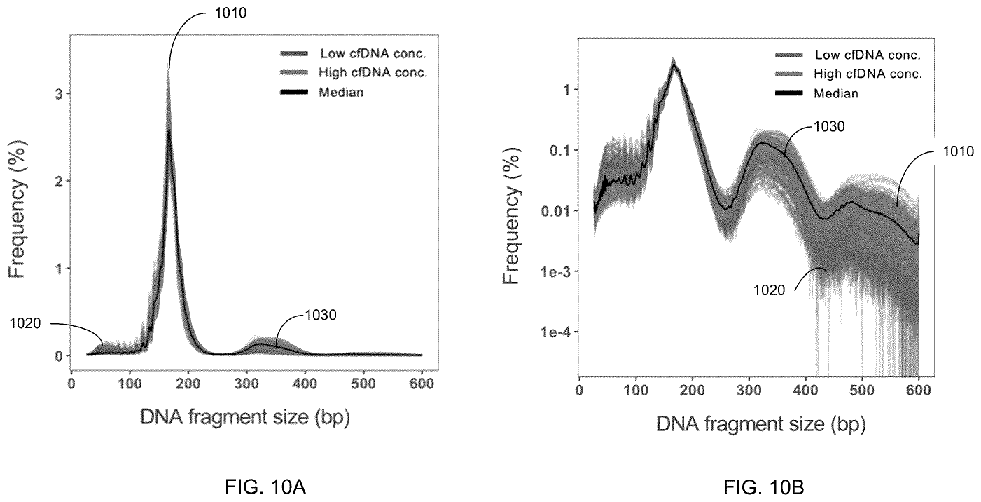

FIGS. 10A-B show the overall size distribution profiles of plasma cfDNA from the highest 10% of individuals (red) 1010 and lowest 10% of individuals (blue) 1020 and median distribution (black) 1030 in terms of cfDNA concentration. Plasma DNA of all subjects exhibited a modal peak size of approximately 166 bp, consistent with reports investigating the mono-nucleosome units of cfDNA. The logarithmic plot (FIG. 10 B) shows the presence of the di- and tri-nucleosome peaks, with a size of approximately 350 bp and 520 bp.

Compared to subjects of low cfDNA concentration, subjects with high cfDNA concentration appeared to have increased frequencies of DNA fragments above 250 bp, with an enhancement at di- and tri-nucleosomal peaks. These results suggested that the subjects with high cfDNA concentrations might exhibit an impaired DNASE1L3-mediated fragmentation process. Short DNA fragments within the size range of 20-120 bp were decreased in subjects with high cfDNA concentrations. These shorter fragments might represent intermediate products (i.e., sub-nucleosomal DNA) derived from mono-nucleosomal DNA during the degradation process.

Subjects with high cfDNA concentration had an increase of larger DNA fragments and a decrease in shorter DNA fragments.

Size distribution plots of different cfDNA concentration ranges (<5, 5-10, 10-15, >15 ng/mL) also show this gradient pattern of decreased frequency of DNA fragments within the 20-120 bp range for samples with higher concentration of total cfDNA, and increased frequency of DNA fragments above 250 bp for the samples with higher total cfDNA concentration.

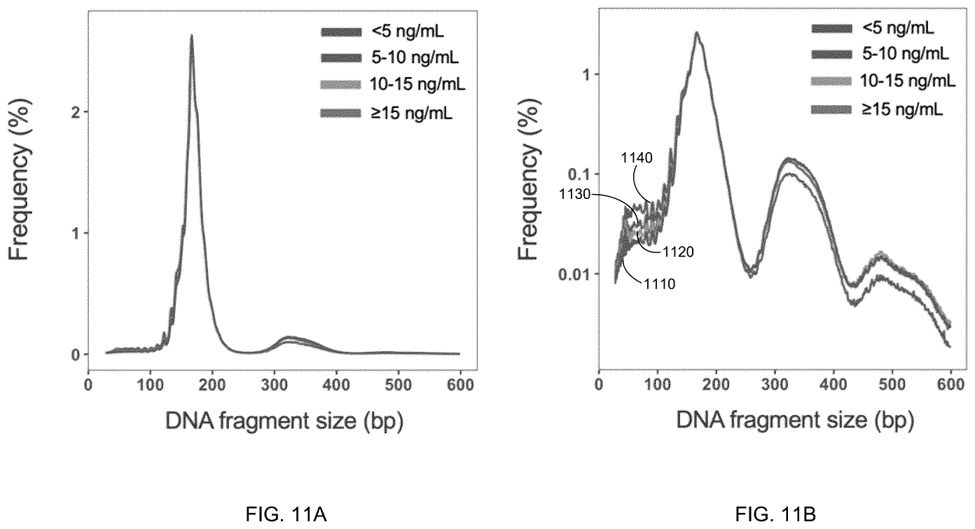

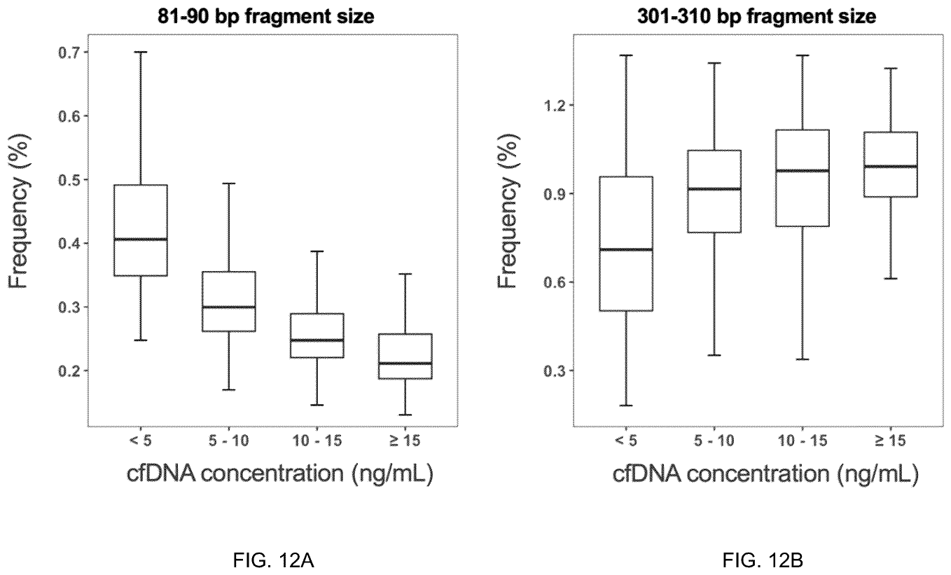

FIGS. 11A-11B and 12A-12B show analysis of size profile of cfDNA with different plasma cfDNA concentration ranges. FIG. 11A shows the mean size profile of plasma DNA fragments plotted on a linear scale. FIG. 11B shows the mean size profile of plasma DNA fragments plotted on logarithmic scale. As can been in both plots but more easily in FIG. 11B, samples with the different plasma cfDNA concentrations have different amounts of short DNA fragments (e.g., less than 160 bp) and differing amounts of long DNA fragments (e.g., greater than 230 bp). The >15 samples 1110 having a concentration equal or higher than 15 ng/ml had the lowest amount of short DNA fragments and the highest amount of long DNA fragments. The 10-15 samples 1120 having a concentration of 10-15 ng/ml had the second lowest amount of short DNA fragments and the second highest amount of long DNA fragments. The 5-10 samples 1130 having a concentration of 5-10 ng/ml had the second highest amount of short DNA fragments and the second lowest amount of long DNA fragments. The <15 samples 1140 having a concentration less than 5 ng/ml had the highest amount of short DNA fragments and the lowest amount of long DNA fragments.

FIG. 12A shows a boxplot of the frequency of cfDNA fragments for the 81-90 bp fragment size range. The vertical axis is the frequency of DNA fragments having a length of 81-90 bp (i.e., amount with that size normalized by the total number of cfDNA fragments). The horizontal axis is the cfDNA concentration, which was measured using fluorescent based quantification (Qubit). In this example, the cfDNA concentration was measured by performing fluorescent based quantification (e.g., using a qubit), although other techniques can be used.

FIG. 12B shows a boxplot of the frequency of cfDNA fragments in the 301-310 bp fragment size range. The vertical axis is the frequency of DNA fragments having a length of 301-310 bp (i.e., amount with that size normalized by the total number of cfDNA fragments).

The horizontal axis is the cfDNA concentration. These plots show the same behavior as plots above, with the higher concentrations having fewer short DNA fragments but having more long DNA fragments.

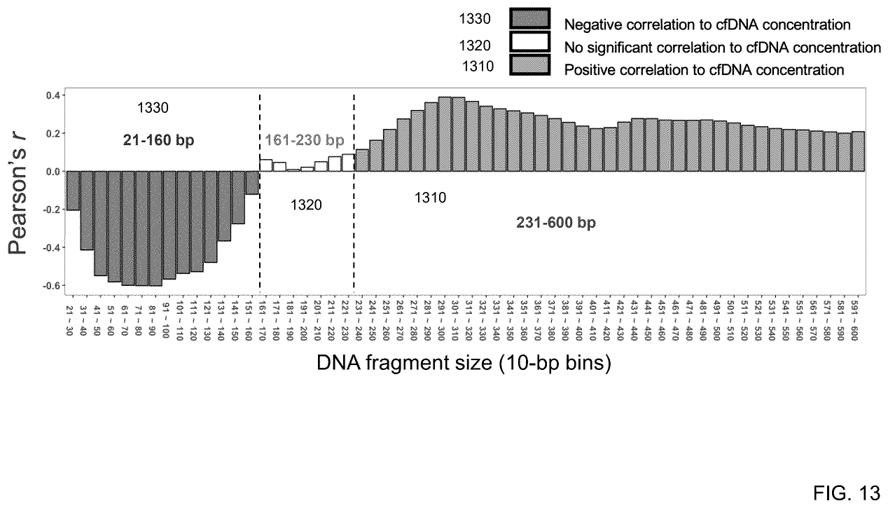

FIG. 13 shows a plot of the correlation between the frequency of DNA fragments within each 10 bp window bin and cfDNA concentration for the 20-600 bp fragment size range, according to embodiments of the present disclosure. The cfDNA frequencies in bins 1330 below 160 bp size (example of short DNA fragments) were found to be negatively correlated to cfDNA concentrations, while bins 1310 above 230 bp size (example of long DNA fragments) were positively correlated to cfDNA concentrations. Bins 1320 had no significant correlation.

The proportional quantification of DNA molecules within certain size ranges may serve as a parameter to approximate the degradation rate of cfDNA in plasma. The process of apoptosis releases DNA with a wide range of sizes (Ungerer et al. 2022; Zhu et al. 2023; Davidson et al. 2024), while subsequent cell-extrinsic cleavage occurs via nucleases in blood, namely DNase1L3 (Serpas et al. 2019). Factors that affect the degradation of cfDNA would alter the proportion of DNA molecules within certain size ranges. The overall larger size profiles in individuals with high cfDNA concentrations might suggest decreased activity of extracellular DNA clearance in blood.

B. Estimation of cfDNA Concentration Using Fragment Size

In some embodiments, cfDNA concentration can be estimated using frequencies of fragment size. The size profile frequencies in all 862 subjects were analyzed. The frequencies of cfDNA fragments for every 10 bp bin size were measured and correlated to cfDNA concentrations. As examples, the frequency of cfDNA between 81-90 bp was negatively correlated to cfDNA concentration (Pearson's r=−0.65, P<0.0001), and the frequency of cfDNA between 301-310 bp was positively correlated (Pearson's r=0.42, P<0.0001).

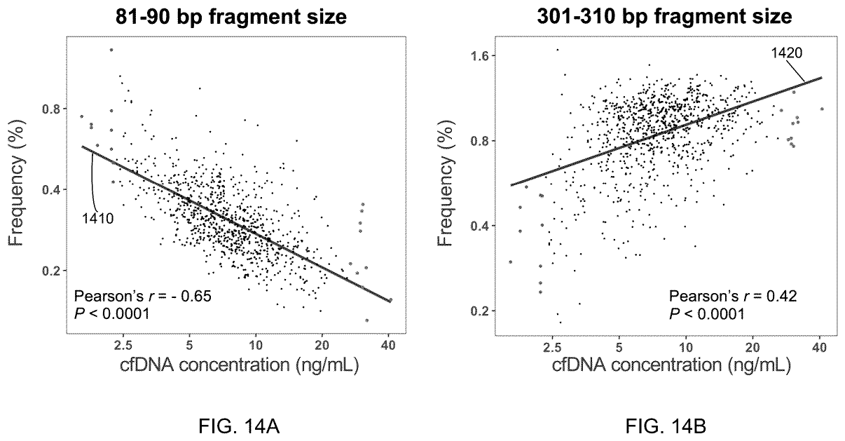

FIG. 14A shows the correlation between the frequency of DNA fragments within each 10 bp window bin and cfDNA concentration for the 81-90 bp fragment size range. FIG. 14B shows the correlation between the frequency of DNA fragments within each 10 bp window bin and cfDNA concentration for the 301-310 bp fragment size range. As can be seen, the amount (e.g., a frequency) of DNA fragments having a size in the given range can generally distinguish between samples that have higher or lower cfDNA concentration. The amount can be normalized. Various normalized amounts could be used, e.g., an amount at a first size (e.g., one size or size range) relative to an amount at a second size (e.g., a different size or size range, which may or may not overlap with the first size). For instance, the second size can be all other sizes or a particular size range, e.g., a range that does not overlap with the first size.

Accordingly, various embodiments can use amounts of DNA fragments having respective sizes to determine the cfDNA concentration of a new sample for which the amount at a certain size is known but the cfDNA concentration has not been measured yet. For example, if an amount of fragments having 81-90 bp is used, a frequency of about 0.3 would correspond to a cfDNA concentration of 10 ng/mL. Such a determination can be made by comparing a measured amount for DNA fragments having a size between 81-90 bp and comparing that amount to one or more calibration data points (e.g., measured frequency and measured cfDNA concentration) having a measured frequency around 0.3. Additionally or alternatively, an amount of fragments having 301-310 bp can be used.

In some embodiments, a calibration function can be used. For example, a calibration function 1410 or 1420 (also referred to as a calibration curve) can be generated (trained) from the training samples (calibration samples) for which cfDNA concentration was measured. Such training samples are shown as the dots in the plots. Each calibration sample has a known concentration of cell-free DNA, e.g., as measured or as a result of manufacture.

### C. Machine Learning Techniques

In some embodiments, machine learning techniques can be used to estimate cfDNA concentration using fragment size. A feature vector can be generated using the amounts (e.g., relative frequencies) of cfDNA fragments of particular sizes or size ranges. A machine learning model can then process the feature vector. Such a feature vector for a size analysis can include an amount of cell-free DNA fragments for one or more sizes. For example, any of the set of amounts in FIG. 10A-12B or 14 can be used. Thus, the sizes can be individual sizes or different size ranges, which may or may not overlap. As other examples, just 1, 2, 3, 4, 5, 6, 7, 8, 9, 10 etc. (or at least one of such numbers) of the sizes (or size ranges) can be used. For instance, just the negatively correlated sizes or the positively corrected sizes can be used. The sizes do not have to be continuous, e.g., ranges of 231-240 and 271-280 can be used. In some embodiments, at least one of the sizes can include a size between 21-160 and 231-600.

Accordingly, in some examples, a range of fragment sizes may be divided into smaller ranges representing bins of amounts (e.g., relative frequencies) of cfDNA fragments within the size range. As an example, fragment sizes may be divided into 5-bp, 10-bp, or 15-bp bins, or other sized bins. A feature vector can be generated using the amounts of cfDNA fragments of particular sizes or size ranges. The feature vector may use various ranges for fragment sizes and may use all or some of the bins.

Examples of machine learning models that may be used for cfDNA concentration estimation can include absolute shrinkage and selection (LASSO), ridge regression, support vector machine (SVM), analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm, etc.

As examples, a model (e.g., a machine learning model) may utilize linear regression, logistic regression, a deep recurrent neural network (e.g., long short-term memory, etc.), a hidden Markov model (HMM), linear discriminant analysis (LDA), k-means clustering, density-based spatial clustering of applications with noise (DBSCAN), random forest algorithm, etc. to predict cfDNA concentration based on fragment size.

In a particular example, a support vector regression (SVR) model can be used to predict cfDNA concentration. The SVR model may integrate some or all input parameters. The model may assign weights for each parameter used and may determine the weights based on feature importance for cfDNA concentration determination. The SVR model may accommodate nonlinear classifications using different kernels of relationship between fragment size and cfDNA concentration. The weighting of parameters may reflect that certain sizes have low feature importance. For example, sizes between 161-230 bp may have low weights associated with them.

A training set can be generated by measuring the values for the feature vector (e.g., the frequencies at the selected sizes) and measuring the cfDNA concentration using an established technique, as will be known to the skilled person. The measured cfDNA concentration is treated as a known value (e.g., as the ground truth). In various implementations, a random selection of 50% of subjects may be used as the training set, and the remaining 50% may be used as a validation set. The model may then be trained and validated using the training set and validation set, respectively. The trained model can then output the predicted cfDNA concentration based on the relative frequencies of the cfDNA fragment sizes.

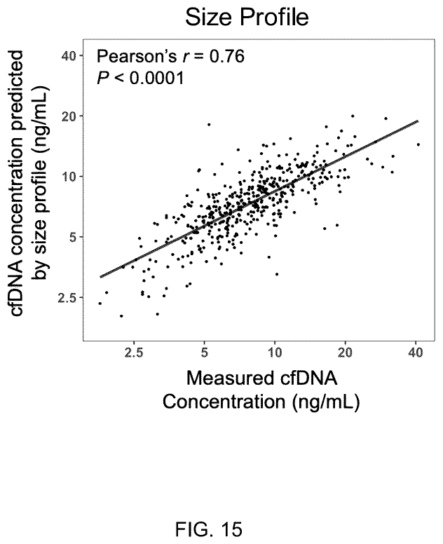

FIG. 15 shows a comparison between measured cfDNA concentration and cfDNA concentration predicted by size profile using a trained SVR model. The vertical axis provides the cfDNA concentration as predicted using the SVR model operating on a feature vector of 581 values corresponding to the frequencies for sizes 20-600. In this example, the cfDNA concentration was measured using a Qubit 4 fluorometer instrument (Invitrogen), although other techniques can be used.

As can be seen, the estimated cfDNA concentration of all DNA fragments (e.g., not just clinically-relevant DNA) provides an accurate estimation of the true cfDNA concentration without having to perform the additional step of measuring. In this manner, an assay that provides genomic sequence information (e.g., PCR or sequencing) can also provide information about the concentration (e.g., per unit volume or mass) of cfDNA.

### D. Method

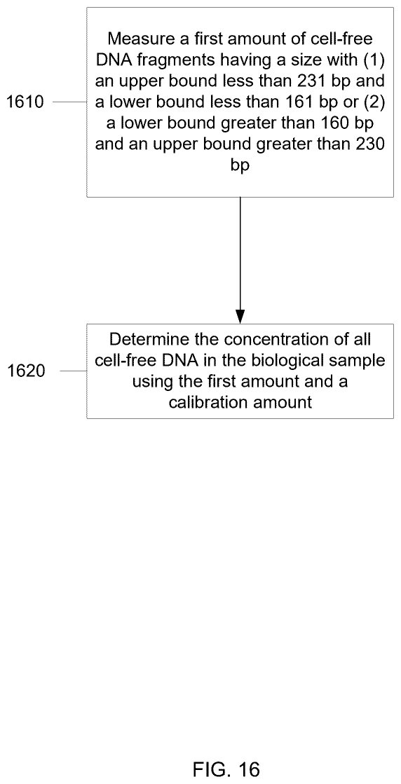

FIG. 16 is a flowchart illustrating a method 1600 for measuring first concentration of all cell-free DNA in a biological sample of a subject, according to some embodiments of the present disclosure. Portions or all steps of method 1600 can be performed by a computer system, including one or more processors. Method 1600 can use a trained ML model that was trained by the computer system or another computer system. The computer system can comprise various devices, e.g., one device that performed the training and another that uses the trained model.

The biological sample may be of various types, e.g., as described herein. The biological sample may be a raw sample (e.g., a blood sample) or a derived sample, e.g., a plasma sample that is derived from a blood sample. In some examples, plasma may be isolated from blood collected from subjects through the use of an isolation kit. For instance, plasma can be isolated from blood using centrifugation, to extract the plasma layer of blood. DNA from plasma can be extracted by silica-based membranes or bead-based methods from isolation kits.

At block 1610, the method 1600 can include measuring a first amount of cell-free DNA fragments in the biological sample having a first size with (1) an upper bound less than 231 bp and a lower bound less than 161 bp or (2) a lower bound greater than 160 bp and an upper bound greater than 230 bp. The upper bound and the lower bound may be the same, resulting in a single size being used. Alternatively, the upper bound can differ from the lower bound, resulting in a size range being used. Thus, the first size may be a size range. Multiple sizes may be used, e.g., in a machine learning model. Thus, a first size can satisfy (1) and a second size can satisfy (2).

The sizes can be individual sizes or different size ranges, which may or may not overlap. As other examples, just 1, 2, 3, 4, 5, 6, 7, 8, 9, 10 etc. (or at least one of such numbers) of the sizes (or size ranges) can be used. For instance, just the negatively correlated sizes or the positively corrected sizes can be used. The sizes do not have to be continuous, e.g., ranges of 231-240 and 271-280 can be used. In some embodiments, at least one of the sizes can include a size between 21-160 and 231-600. As a further example for (1), the upper bound can be any value between 230 bp and 22 bp. As a further example for (2), the lower bound can be any value between 161 and 600.

The measurement can be performed in various ways, e.g., in aggregate or by analyzing individual DNA fragments. Aggregate measurements can be performed via a physical separation method (e.g., electrophoresis) or biochemical assay, such as real-time PCR. Thus, the purified DNA could be assessed using electrophoretic methods, which are techniques to separate DNA fragments on the basis of size. As an example, electrophoresis may be used to measure an amount the plurality of cfDNA fragments of a particular size. Such captured cfDNA fragments of a particular size can be quantified using an intensity the plurality of cfDNA fragments corresponding to that particular size, such as by using real-time PCR. Thus, in some examples, the intensity can be indicative of relative amount of cfDNA fragments having an estimated size or size range. For a biochemical assay, different primers corresponding to different lengths of DNA fragments can be used with different probes specific to a part of the genome that only occurs when primers corresponding to a particular size are used.

The analysis of individual DNA fragments can be performed using a biochemical assay (e.g., digital PCR) or a combination assay that uses physical and in silico aspects, e.g., sequencing and alignment. In such an embodiment, one or more sequence reads can be received for each cfDNA fragment, and using the one or more sequence reads to determine the size of each cfDNA fragment. As examples, the sequence reads can be generated from paired-end sequencing, single-molecule sequencing, targeted sequencing, or the like, as well as probe-based techniques. The sequence reads can be analyzed, aligned with a reference genome, combined with other data (e.g., paired-end data), or a combination thereof to estimate the size of each cfDNA fragment. In one example, the sequence reads can be paired-end sequence reads, and using the one or more sequence reads to determine the size of the cell-free DNA fragment can include aligning the paired-end sequence reads to a reference sequence. Once aligned, a distance between at least two positions on the reference genome that correspond to each paired-end sequence read can be used to determine the size of each cfDNA fragment.

An amount of cell-free DNA fragments in a biological sample having a particular size can be normalized, e.g., a frequency. Such normalization may involve scaling (e.g., by multiplication or division) an initial amount, such as a count or intensity. As examples, such normalization can use a sample size, e.g., a total number of cfDNA fragments (as may be done implicitly by analyzing a fixed number of cfDNA fragments), a total amount of genomic material, a total volume, or a reference measurement from a known sample. Accordingly, the first amount may be normalized by a total number of the plurality of cell-free DNA fragments or any one of these other options may be used.

At block 1620, the first concentration of all cell-free DNA in the biological sample is determined using the first amount and one or more calibration amounts of cell-free DNA fragments having the first size determined from one or more calibration samples. Each calibration sample has a known concentration of cell-free DNA, e.g., as measured or as a result of manufacture. The calibration sample(s) can be from healthy subjects and/or from subjects with a condition (e.g., pregnancy or cancer).

The first concentration can be a category, e.g., high or low, or any number of classification of varying degree (level), such as out of at least 3, 4, 5, 6, 7, 8, 9, 10, or more levels. The categories can correspond to a range of values for the first concentration, e.g., a range of values for mass per volume. In other examples, the first concentration can be a numerical value, e.g., specified as a particular numerical value for a mass per volume.

Various measurement techniques may be used. As an example, a device such as a qubit may be used with a fluorescent dye to integrate into the double-stranded DNA and measure cfDNA. Other types of spectrophotometers or other measurement devices can be used.

In some embodiments, determining the first concentration of cell-free DNA in the biological sample includes comparing the first amount to the one or more calibration amounts. Such a comparison can use a calibration function, e.g., as shown in FIGS. 14A and 14B.

In some examples, the one or more calibration amounts are a plurality of calibration amounts and the one or more calibration samples are a plurality of calibration samples. The method 1600 may further include measuring other amounts of cell-free DNA fragments having other sizes. The first concentration of cell-free DNA in the biological samples can be determined using a machine learning model that operates on the first amount and the other amounts. The machine learning model may be trained using a training set that includes calibration amounts and known concentrations of the calibration samples. A feature vector derived from the measured cell-free DNA fragments sizes and frequencies may be inputted into the machine learning model. In a particular example, an SVR model may be used to determine the concentration.

In some examples, the first size and the other sizes can include all sizes within a range of 21 bp to 600 bp. In other examples, the first size and the other sizes can include all sizes within a range of 21 bp to 160 bp and 231 bp to 600 bp but not between 161 bp to 230 bp. Any subranges between these specified ranges may be included or excluded as indicated. Such usage may follow from FIG. 13 and the corresponding description.

The first size and the other sizes can include a plurality of size ranges, e.g., as depicted in FIG. 13. Each size range may be of a specified width, e.g. 10 bp or other values, such as those mentioned herein. The first amount and the other amounts may each be determined for one of the plurality of size ranges.

As described above, various sizes can be used. Additionally, other types of measurements can be used, e.g., end motifs as described in the next section. Accordingly, method 1600 may further include measuring a second amount of cell-free DNA fragments in the biological sample having a set of one or more sequence motifs corresponding to ending sequences of the cell-free DNA fragments. Determining the first concentration of all cell-free DNA in the biological sample may further use the second amount and one or more additional calibration amounts determined from one or more additional calibration samples, having a known concentration of cell-free DNA. The one or more additional calibration samples for the end motifs can be the same as the one or more calibration samples for the sizes, as would be done when both are part of a feature vector used as input to an ML model.

In some embodiments as part of a workflow, an initial part (first portion) of the sample can be analyzed to determine the total cfDNA concentration of that first portion. Clinical samples with low cfDNA concentration can still sequenced and analysed if there is sufficient plasma volume remaining. For example, the cfDNA concentration can be used along with the remaining volume to estimate the amount of cfDNA present. If the remaining cfDNA is greater than a threshold, then an assay (e.g., sequencing) can be performed on the remaining volume. If the cfDNA concentration is too low and plasma volume is scarce (e.g., estimated remaining cfDNA is below a threshold), embodiments may opt to not perform the assay on the remaining volume as there will not be sufficient data for subsequent analysis. Thus, the plurality of cfDNA fragments may be for the first portion, and then an assay can be further performed on a second portion of the cfDNA fragments from the rest of the sample.

In some examples, determining the first concentration of all cell-free DNA in the biological sample can include comparing the first amount to the one or more calibration amounts. The comparison of the first amount to the one or more calibration amounts may use a calibration function. In some examples, the first concentration may have a unit of mass per volume. In some examples, the first size is a size range.

The method 1600 may further include comparing the first concentration to a threshold. If the first concentration exceeds the threshold, the method 1600 may include determining a property of the biological sample based on an analysis of cell-free DNA fragments of the biological sample. The plurality of cell-free DNA fragments may be from a first portion of the biological sample. Cell-free DNA fragments of a second portion of the biological sample may be analyzed to determine the property of the biological sample. In some examples, the plurality of cell-free DNA fragments used to determine the first concentration are used to determine the property of the biological sample.

### III. END MOTIF ANALYSIS

Additionally or alternatively, end motifs can be used to determine a concentration of all cfDNA in a biological sample. Certain end motifs can be used, as described below. Different end motifs can be positively or negatively related to changes in cfDNA concentration. Various embodiments can use one or more end motifs that are positively related, one or more end motifs that are negatively related, or use some of both. A general description of end motifs is provided, followed by an analysis of quantities of which end motifs change in relation to changes in cfDNA concentration, with examples of machine learning.

### A. Example End Motifs

An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

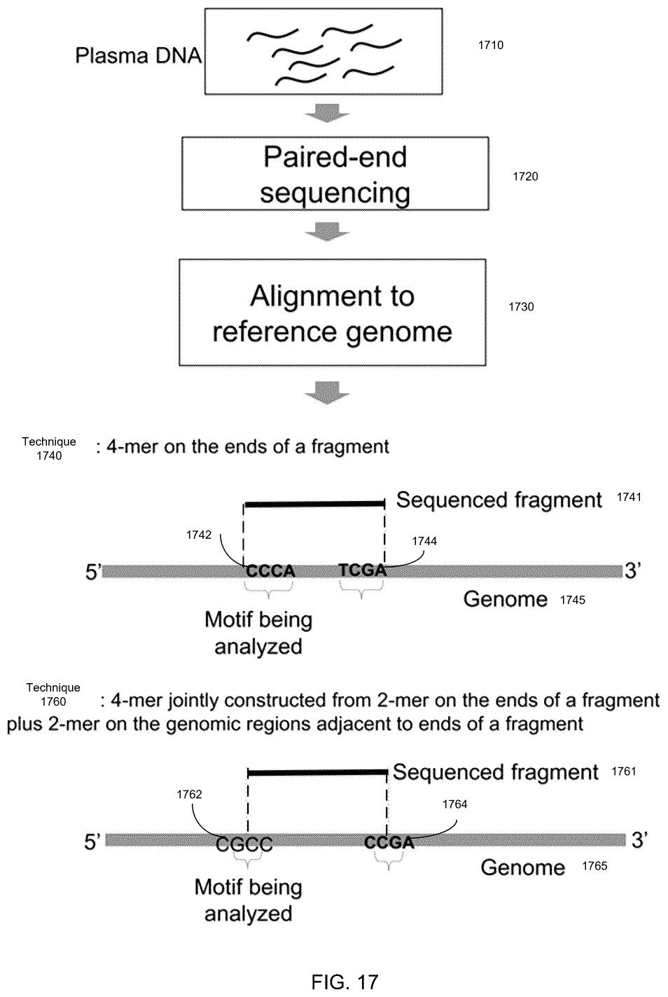

FIG. 17 shows examples for end motifs according to embodiments of the present disclosure. FIG. 17 depicts two ways to define 4-mer end motifs to be analyzed. In technique 1540, the 4-mer end motifs are directly constructed from the first 4-bp sequence on each end of a plasma DNA molecule. For example, the first 4 nucleotides or the last 4 nucleotides of a sequenced fragment could be used. In technique 1560, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, and 7-mer end motifs.

As shown in FIG. 17, cell-free DNA fragments 1710 are obtained, e.g., using a purification process on a blood sample, such as by centrifuging. Besides plasma DNA fragments, other types of cell-free DNA molecules can be used, e.g., from serum, urine, saliva, and other such cell-free samples mentions herein. In one embodiment, the DNA fragments may be blunt-ended.

At block 1720, the DNA fragments are subjected to paired-end sequencing, although the entire DNA fragment can be sequenced. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment.

At block 1730, the sequence reads can be aligned to a reference genome. This alignment is to illustrate different ways to define a sequence motif, and may not be used in some embodiments. The alignment procedure can be performed using various software packages, such as BLAST, FASTA, Bowtie, BWA, BFAST, SHRiMP, SSAHA2, NovoAlign and SOAP.

Technique 1740 shows a sequence read of a sequenced fragment 1741, with an alignment to a genome 1745. With the 5′ end viewed as the start, a first end motif 1742 (CCCA) is at the start of sequenced fragment 1741. A second end motif 1744 (TCGA) is at the tail of the sequenced fragment 1741. Such end motifs might, in one embodiment, occur when an enzyme recognizes CCCA and then makes a cut just before the first C. If that is the case, CCCA will preferentially be at the end of the plasma DNA fragment. For TCGA, an enzyme might recognize it, and then make a cut after the A.

Technique 1760 shows a sequence read of a sequenced fragment 1761, with an alignment to a genome 1765. With the 5′ end viewed as the start, a first end motif 1762 (CGCC) has a first portion (CG) that occurs just before the start of sequenced fragment 1761 and a second portion (CC) that is part of the ending sequence for the start of sequenced fragment 1761. A second end motif 1764 (CCGA) has a first portion (GA) that occurs just after the tail of sequenced fragment 1761 and a second portion (CC) that is part of the ending sequence for the tail of sequenced fragment 1761. Such end motifs might, in one embodiment, occur when an enzyme recognizes CGCC and then makes a cut in between the G and the C. If that is the case, CC will preferentially be at the end of the plasma DNA fragment with CG occurring just before it, thereby providing an end motif of CGCC. As for the second end motif 164 (CCGA), an enzyme can cut between C and G. If that is the case, CC will preferentially be at the end of the plasma DNA fragment. For technique 1760, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

The higher the number of nucleotides included in the cell-free DNA end signature, the higher the specificity of the motif because the probability of having 6 bases ordered in an exact configuration in the genome is lower than the probability of having 2 bases ordered in an exact configuration in the genome. Thus, the choice of the length of the end motif can be governed by the needed sensitivity and/or specificity of the intended use application.

As the ending sequence is used to align the sequence read to the reference genome, any sequence motif determined from the ending sequence or just before/after is still determined from the ending sequence. Thus, technique 1760 makes an association of an ending sequence to other bases, where the reference is used as a mechanism to make that association. A difference between techniques 1740 and 1760 would be to which two end motif a particular DNA fragment is assigned, which affects the particular values for the relative frequencies. But, the overall result (e.g., fractional concentration of clinically-relevant DNA, classification of a level of pathology, etc.) would not be affected by how the a DNA fragment is assigned to an end motif, as long as a consistent technique is used for the training data as used in production.

The counted numbers of DNA fragments having an ending sequence corresponding to a particular end motif may be counted (e.g., stored in an array in memory) to determine relative frequencies. As described in more detail below, a relative frequency of end motifs for cell-free DNA fragments can be analyzed. Differences in relative frequencies of end motifs have been detected for different types of tissue and for different phenotypes, e.g., different levels of pathology. The differences can be quantified by an amount of DNA fragments having specific end motifs or an overall pattern, e.g., a variance (such as entropy, also called a motif diversity score), across a set of end motifs (e.g., all possible combinations of the k-mers corresponding to the length used).

### B. Occurrence of End Motifs for Low and High Concentration Samples

We investigated whether the distribution of 5′ end motifs varied with cfDNA concentration. To this end, we systematically studied the correlations of 256 end motifs (4-mer) to the plasma cfDNA concentrations. Other sizes of end motifs can be used.

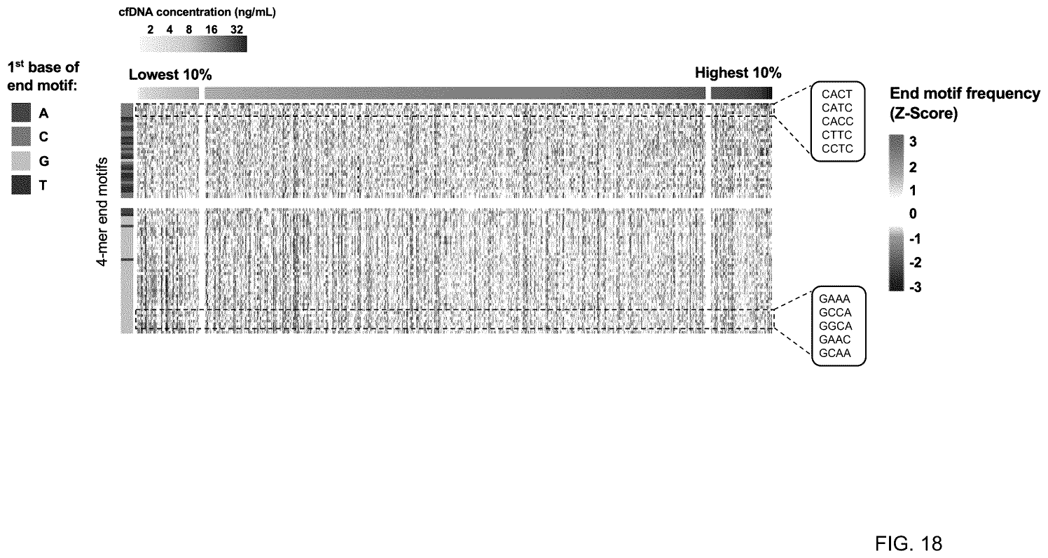

FIG. 18 is a heatmap analysis with rows indicating a particular 4-mer motif, the first base of the motif highlighted by a specific color in the left-most column (A, C, G and T colored by green, red, yellow, and blue). The heatmap analysis visualizes the pattern of the significantly correlated motifs to cfDNA concentration. Each column indicates plasma DNA sample from one subject with cfDNA concentration increasing left to right. Each row corresponds to a particular 4-mer motif, with the composition of the first base indicated, while columns represent plasma DNA from each subject. For a better visualization, column-wise normalization (Z-score) was applied to the motif frequencies. The frequency Z-score, calculated for each end motif, is shown by the color scale.

The heatmap shows end motifs with significant correlation to cfDNA concentration. The frequency of all 256 end motifs (4-mer) were correlated to the cfDNA concentration. As shown, a gradual change of motif frequencies was observed across the whole spectrum of cfDNA concentrations, with 4-mer end motifs starting with a 5′ C nucleotide appearing to gradually decrease as the cfDNA concentration increased. However, the 4-mer end motifs starting with a 5′ G nucleotide showed a rising gradation. A total of 79 end motifs were found to be significantly correlated with cfDNA concentrations, with 34 motifs being negatively correlated and 46 being positively correlated after adjustment for multiple comparisons by Bonferroni's correction.

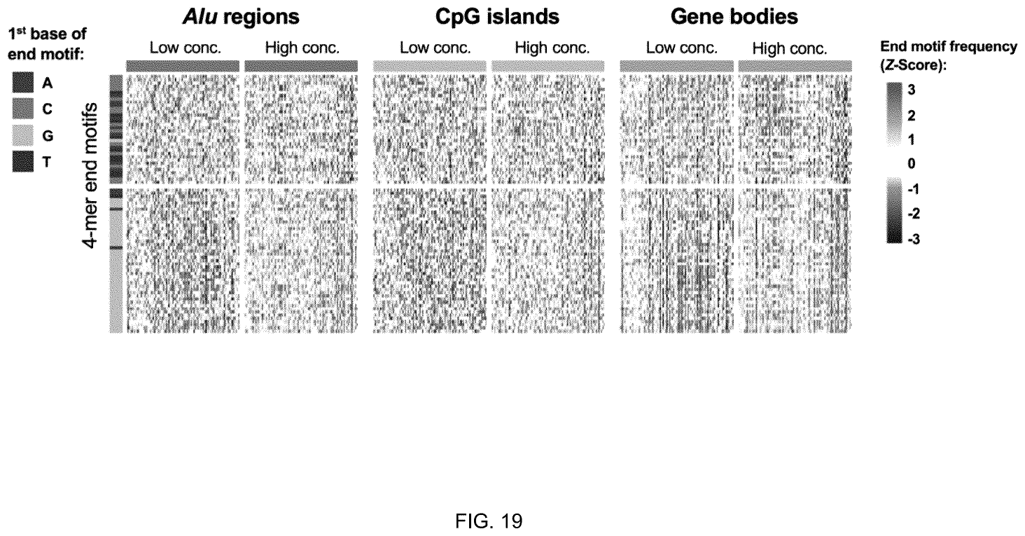

FIG. 19 shows a heatmap analysis showing z-scores of informative end motif frequencies between lowest and highest 10% of subjects across different genomic regions (Alu regions, CpG islands and gene bodies). A sequence context-based normalization method (O/E ratio—observed to expected end motif frequency) was used to minimize the potential biases in end motif analysis across different genomic regions. For example, a normalized end motif frequency can be calculated as a ratio of observed and expected frequencies (e.g., for a particular region) and then divided by the sum of the set (e.g., all 256 for 4-mers) of normalized motif frequencies. The total normalized end motif frequency can be equal to 100%.

By evaluating the highest and lowest 10% of individuals in the studied cohort, the selected 79 significantly correlated motifs also showed a consistent trend when sub-classified into Alu regions, CpG islands and gene body regions, suggesting that these changes in end motif profiles appear to be present across different genomic regions.

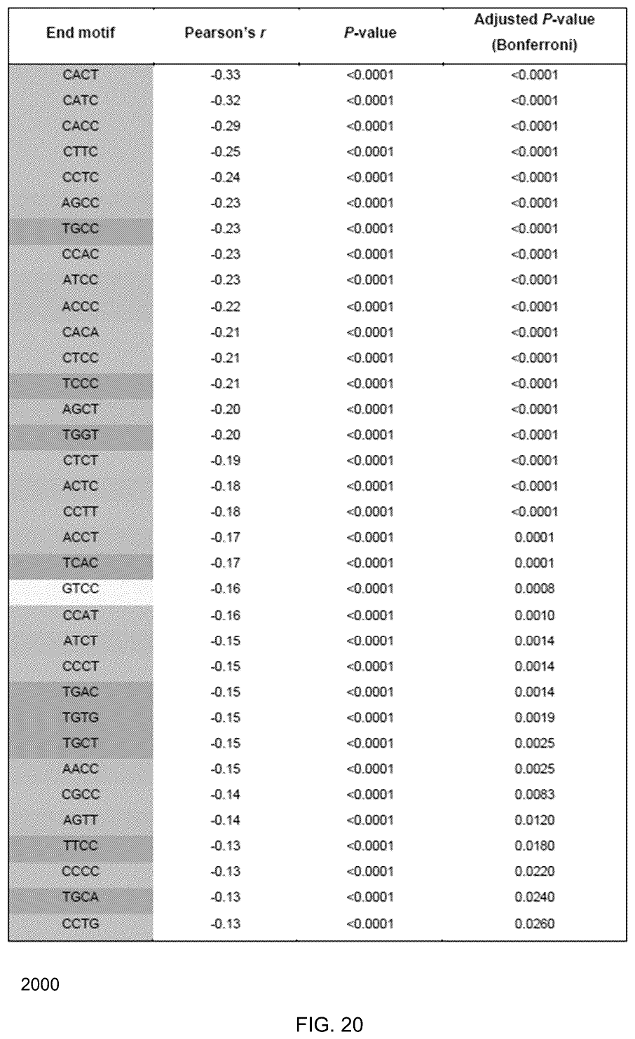

FIG. 20 is a table 2000 listing 4-mer end motifs with a significant negative correlation to cfDNA concentration in the studied cohort, according to embodiments of the present disclosure. Motifs with a negative correlation were mostly C-end motifs (such as CACT, CATC, CACC), with several T-end and A-end motifs. In various embodiments, a set of one or more sequence motifs can be selected from the group consisting of a top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs listed in table 2000.

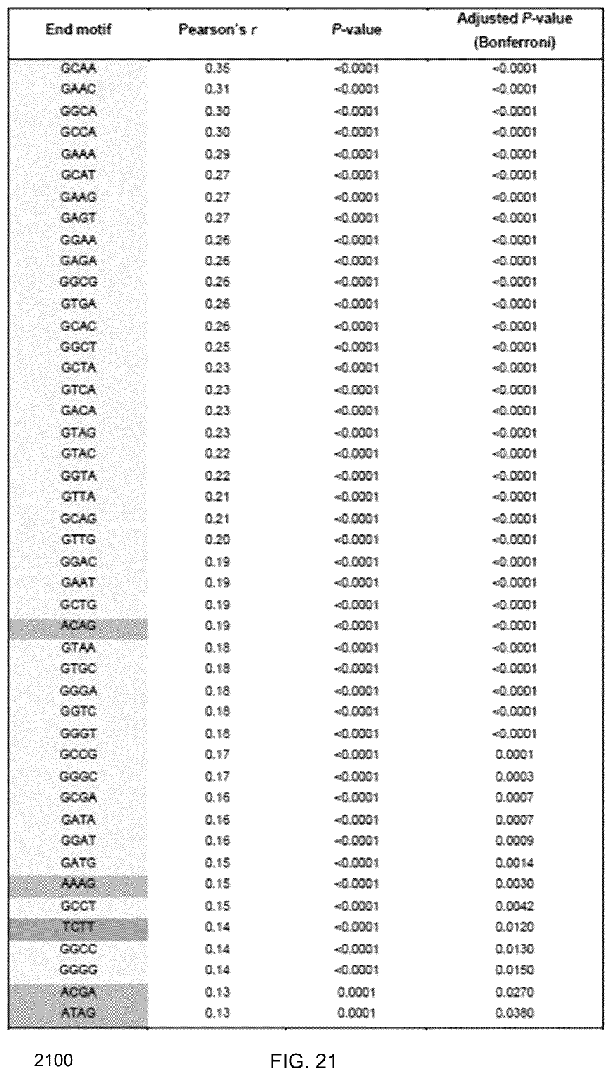

FIG. 21 is a table 2100 listing 4-mer end motifs with a significant positive correlation to cfDNA concentration in the studied cohort, according to embodiments of the present disclosure. The positively correlated motifs were nearly all G-end motifs (such as GCAA, GAAC, GGCA). In various embodiments, a set of one or more sequence motifs can be selected from the group consisting of a top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs listed in table 2100.

Table 1 in section X provides a listing of all of the 256 4-mer end motifs with corresponding p-values for positive and negative correlation. In various embodiments, a set of one or more sequence motifs can be selected from the group consisting of a top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs listed in either tables 1, 2000, or 2100. That is, the end motifs with the top N values can be selected, where N is any of the values provided here. The top values can be those with the lowest values p-value or highest absolute Pearson r value as determined for the training set or the testing set.

Similarly, the deconvolutional analysis of the end motifs revealed the increased contribution of Profile V (See section IV. B) in subjects with higher cfDNA concentrations, which is a cleavage profile characterized by a series of G-end motifs.

### C. Machine Learning Techniques

As with the size analysis, machine learning techniques can be used to estimate cfDNA concentration using end motifs. A feature vector can be generated using the amounts (e.g., relative frequencies) of cfDNA fragments of particular end motifs. A machine learning model can then process the feature vector. The same types of machine learning models for size can be used for the end motifs.

Motivated by the gradation patterns observed in the heatmap, we explored whether we could use regression to model the relationship between cfDNA concentration and 4-mer end motifs. We adopted the support vector regression (SVR) model, with a random selection of 50% of subjects used as the training set, and the remaining 50% as validation. Accordingly, in a particular example, a support vector regression (SVR) model can be developed for predicting cfDNA concentration based on the relative frequencies of each end motif. A feature vector based on the relative frequencies may be inputted into the SVR model. The SVR model may integrate some or all input parameters. The weights for each parameter may be determined based on feature importance in terms of cfDNA concentration determination. The SVR model may accommodate nonlinear classifications using different kernels of relationship between end motif and cfDNA concentration. The weighting of parameters may reflect that certain end motifs have low feature importance. For example, sequence motifs not listed in FIGS. 20 and 21 may have low weights associated with them. Having a sufficiently low weight is equivalent to not using a sequence motif at all.

A training set can be generated by measuring the values for the feature vector (e.g., the frequencies of selected end motifs) and measuring the cfDNA concentration using an established technique. The measured cfDNA concentration is treated as a known value (e.g., as the ground truth). In some embodiments, a random selection of 50% of subjects may be used as the training set, and the remaining 50% may be used as a validation set. The model may then be trained and validated using the training set and validation set, respectively. The trained model can then output the predicted cfDNA concentration based on the relative frequencies of the end motifs.

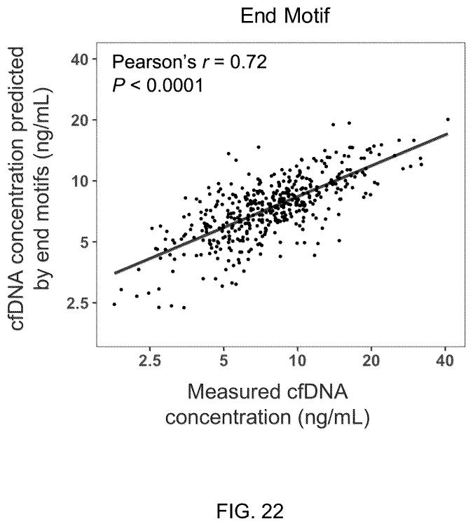

FIG. 22 shows a plot between the cfDNA concentration predicted by SVR and the cfDNA concentration measured by fluorometric quantification in the validation set (Pearson's r=0.72, P<0.0001). FIG. 22 uses all 256 end motifs for 4-mers. However, other lengths of sequence motifs can be used, and a smaller set of end motifs can be used, e.g., as listed in tables 2000 and 2100, or from the top N end motifs from table 1. The top values can be those with the lowest values p-value or highest absolute Pearson r value as determined for the training set or the testing set.

Quantification of the plasma cfDNA concentration was performed using fluorometric measurements. Fluorometric methods measure the total DNA concentration, including DNA from both nuclear and mitochondrial origins. The readings from fluorometric measurements (n=25) were well correlated with results obtained from the droplet digital PCR assay, which targeted a single-copy gene valosin-containing protein (VCP).

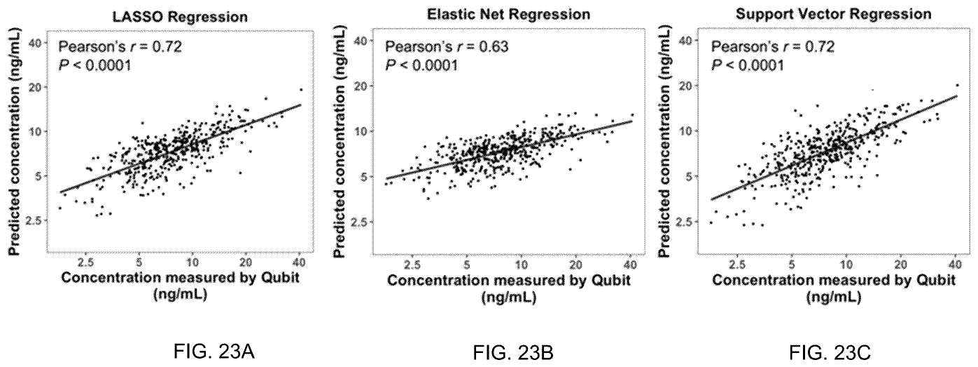

FIG. 23A shows the correlation between measured cfDNA concentration and cfDNA concentration predicted by end motifs using LASSO regression, according to embodiments of the present disclosure. FIG. 23B shows the correlation between measured cfDNA concentration and cfDNA concentration predicted by end motifs using elastic net regression, according to embodiments of the present disclosure. FIG. 23C shows the correlation between measured cfDNA concentration and cfDNA concentration predicted by end motifs using support vector regression, according to embodiments of the present disclosure.

### D. Use of Different Numbers of End Motifs

To investigate whether a selection of certain end motifs could result in more accurate total plasma cfDNA concentration predictions compared to a random selection of end motifs, an SVR model of total cfDNA prediction was trained in two ways using the EBV positive cohort of 862 individuals with 50% training and 50% testing. SVR models were trained on a ranked order of end motifs or a random selection of end motifs. The ranked order of end motifs included the top 5, 10, 20, 40, 80, 160, or 256 end motifs ordered by P-value of the correlation of individual end motifs to total plasma cfDNA concentration as listed in Table 1. The random selection of end motifs included random selections of 5, 10, 20, 40, 80, 160, or 256 end motifs. 100 random selections were performed for each number of end motifs. Samples used for training and testing were kept consistent (i.e., the same n=431 samples were used for training, and the remaining n=431 were used for testing).

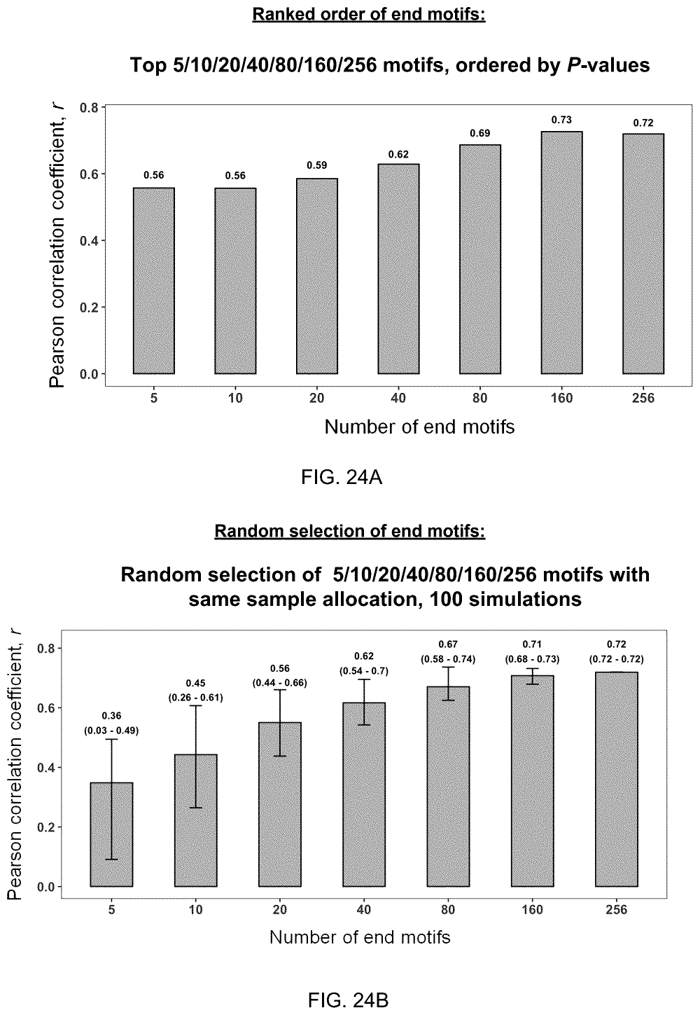

FIGS. 24A-24B show bar plots of correlation between measured and predicted total plasma cfDNA concentration by an SVR model trained using different numbers of end motifs. FIG. 24A shows a bar plot of measured and predicted concentration using a ranked order of end motifs. FIG. 24B shows a bar plot of measured and predicted concentration using a random selection of end motifs.

As shown in FIGS. 24A-24B, an SVR model constructed on ranked selections of end motifs has greater Pearson's r coefficient values compared to randomly selected end motifs for a given number of end motifs used for model construction. In particular, this was observed in models constructed using fewer end motifs. For example, the mean Pearson'r for ranked selection of five motifs was 0.56, while the mean Pearson's r for random selection of five motifs was 0.36. Furthermore, random selections of end motifs generally resulted in high variability in correlation strength. For example, random selection of only five end motifs produced a Pearson's r value ranging from 0.03-0.49.

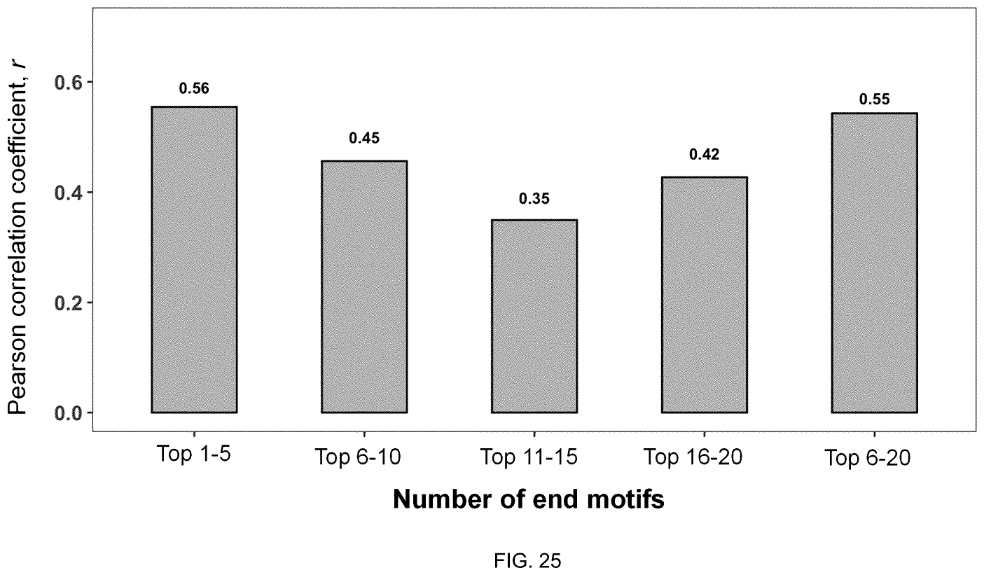

FIG. 25 shows a bar plot of the correlation between measured and predicted total plasma cfDNA concentration using the top 1-5, top 6-10, top 11-15, top 16-20, and top 6-20 ranked end motifs. The accuracy of the top 1-5 motifs is comparable to that of using the top 6-20 motifs. For total cfDNA concentrations, the Pearson's r for the top one motif (GCAA) was 0.37 in the testing set (0.35 in the training set), and the top two motifs (GCAA and CACT) was 0.53 in the testing set; which is quite comparable to the results for the top 5 motifs (Pearson's r=0.56) in the testing set.

### E. Method

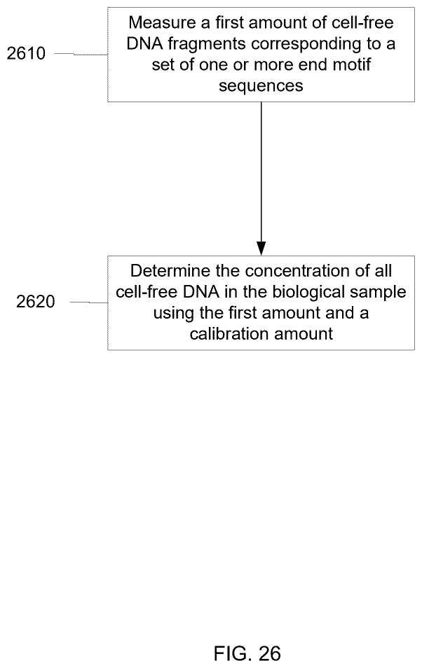

FIG. 26 is a flowchart illustrating a method 2600 for measuring first concentration of all cell-free DNA in a biological sample of a subject, according to some embodiments of the present disclosure. Portions or all steps of method 2600 can be performed by a computer system, including one or more processors. Method 2600 can use a trained ML model that was trained by the computer system or another computer system. The computer system can comprise various devices, e.g., one device that performed the training and another that uses the trained model.

Certain steps or procedures performed in method 2600 can be performed in a similar manner as method 1600.

At block 2610, the method 2600 can include measuring a first amount of a plurality of cell-free DNA fragments in the biological sample having a set of one or more sequence motifs corresponding to ending sequences of the plurality of cell-free DNA fragments. The set of one or more sequence motifs can be a set of sequence motifs.

An amount of cell-free DNA fragments in a biological sample having a particular one or more end motifs can be normalized, e.g., a frequency. Such normalization may involve scaling (e.g., by multiplication or division) an initial amount, such as a count or intensity. As examples, such normalization can use a sample size, e.g., a total number of cfDNA fragments (as may be done implicitly by analyzing a fixed number of cfDNA fragments), a total amount of genomic material, a total volume, or a reference measurement from a known sample.

The amount can be an aggregate of all of the sequence motifs in the set of one or more sequence motifs (also referred to as end motifs). In other embodiments, an amount can be determined for each sequence motif in the set, where the set of amounts can be used, e.g., in a machine learning model. Example sequence motifs can be found in Tables 2000 and 2100 of FIGS. 20-21 and in Table 1, or any combination or portions thereof. Example sequence motifs can include 3-mers related to 4-mers listed. For example, GCAA, GCAC, GCAG, and GCAT all appear in the top 80 end motifs of Table 1, and thus using an amount of GCA is related to the these 4-mers. A 3-mer could be used as long as at least one, two, or three of the related 4-mers are present. The same goes for other tables herein.

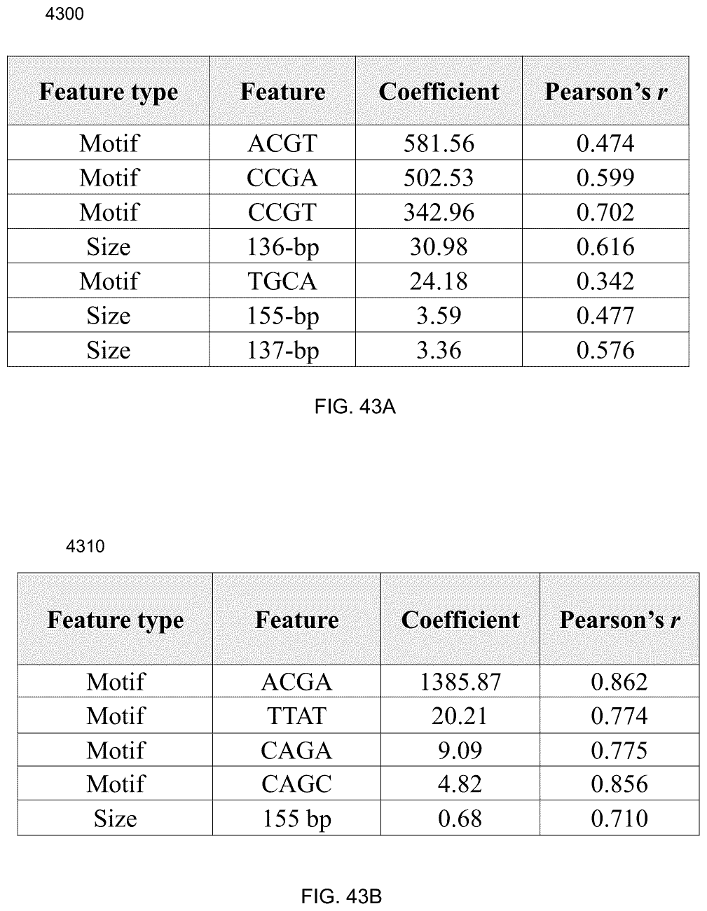

These tables list the end motifs in order starting with the most significant correlation at the top. In various embodiments, the top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80 or more of the end motifs from either, any two of, or all three tables can be used. For example, the set of one or more sequence motifs can be selected from a group consisting of a top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs listed in Table 1 for lowest p-value or highest Pearson r value as determined for the training set or the testing set. The selection of the top end motifs can be selected based on values across all three tables as well as tables 4300 and 4400 in FIGS. 43A and 43B. Accordingly, a top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs of Table 1, 2000, 2100, 4300, 4400, or a combination thereof can be used. In some implementations, all of the end motifs of a certain length can be used, e.g., all 64 3-mer end motifs or all 256 4-mer end motifs. End motif pairs can be used in addition or alternatively to single ended motifs.

The measurement can be performed in various ways, e.g., via sequencing or probe-based techniques such as PCR. One or more sequence reads can be received for each cfDNA fragment, where the one or more sequence reads are used to determine one or more end motifs of each cfDNA fragment. In other implementations, an intensity signal of a probe hybridizing to a particular end motif can provide an amount of the particular end motif. Aspects of block 2610 can be performed in a similar manner as block 1610 of method 1600.

At block 2620, the first concentration of all cell-free DNA in the biological sample is determined using the first amount and one or more calibration amounts of cell-free DNA fragments having the set of one or more sequence motifs determined from one or more calibration samples. Each calibration sample has a known concentration of cell-free DNA, e.g., as measured or as a result of manufacture. Various measurement techniques may be used. As an example, a device such as a qubit may be used with a fluorescent dye to integrate into the double-stranded DNA and measure cfDNA. Other types of spectrophotometers or other measurement devices can be used.

In some examples, method 2600 may determine the first concentration of cell-free DNA in the biological samples using a machine learning model that operates on the first amount and other amounts. The machine learning model may be trained using a training set that includes calibration amounts and known concentrations of the calibration samples, e.g., as described herein. A feature vector derived from the measured end motif frequencies may be inputted into the machine learning model. In a particular example, an SVR model may be used to determine the concentration. In some examples, the first concentration may have a unit of mass per volume.

The one or more calibration samples can be a plurality of calibration samples, and the one or more calibration amounts can be a plurality of calibration amounts, e.g., when training a machine learning model such as an SVR model, a linear regression model, or other models described herein. The method 2600 can further include measuring other amounts of cell-free DNA fragments having other sequence motifs corresponding to ending sequences of the plurality of cell free DNA fragments. Determining the first concentration of all cell-free DNA in the biological sample can include using a machine learning model that operates on the first amount and the other amounts. The machine learning model may be trained using a training set that include the plurality of calibration amounts and known concentrations of the plurality of calibration samples.

In some examples, determining the first concentration of all cell-free DNA in the biological sample can include comparing the first amount to the one or more calibration amounts, e.g., when the first concentration is a category, such as high or low concentration as described herein. For greater resolution, the comparison of the first amount to the one or more calibration amounts may use a calibration function determined using the plurality of calibration amounts and the known concentrations. Comparing the first amount to the one or more calibration amounts may be performed via input of the first amount into the calibration function determined from the plurality of calibration amounts. In such examples, the first concentration can be a numerical value, e.g., specified as a particular numerical value for a mass per volume.

The method 2600 may further use the size analysis combined with the end motif analysis, e.g., as described in section IV. For example, a second amount of cell-free DNA fragments having a first size in the biological sample can be measured. As an example option, the first size can have an upper bound less than 231 bp and a lower bound less than 161 bp. As another example option, the first size can have a lower bound greater than 160 bp and an upper bound greater than 230 bp. Determining the first concentration of all cell-free DNA in the biological sample may further use the second amount and additional calibration amounts determined from additional calibration samples, each having a known concentration of cell-free DNA. In some examples, the calibration sample(s) may be the additional calibration sample(s). The first size can be a size range.

As further described in section IV.B, an F-profile that includes the first amount can be used, e.g., when a set of sequence motifs is used. For example, method 2600 may further include storing a set of reference F-profiles. Each reference F-profile of the set of reference F-profiles may identify a proportion of cell-free DNA fragments having the same sequence motif for each sequence motif of the set of sequence motifs. Each reference F-profile may be associated with a type of fragmentation factors. A sample end-motif profile can be determined by measuring other amounts of cell-free DNA fragments having other sequence motifs corresponding to ending sequences of the plurality of cell-free DNA fragments. The sample end-motif profile can include the first amount and the other amounts. The method 2600 may further include determining proportional contributions of the set of reference F-profiles whose proportional aggregation provide the sample end-motif profile. In some examples, the proportional contributions sum to one. Then, determining the first concentration of all cell-free DNA in the biological sample can use a first proportional contribution of a first reference F-profile of the set of reference F-profiles and one or more reference contributions determined from the one or more calibration samples.

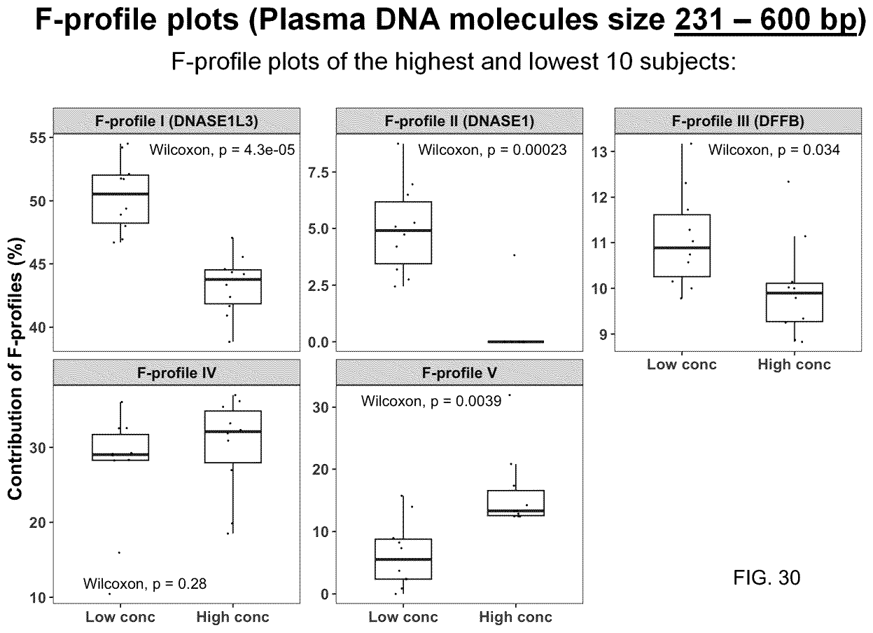

The determination of the first concentration may include comparing the first proportional contribution to the one or more reference contributions, e.g., as described FIG. 30, as may be done to classify a sample as having a low or high concentration or other levels of concentration. In implementations, where the one or more reference contributions may be a plurality of reference contributions. In such examples, comparing the first proportional contribution to the one or more reference contributions can use a calibration function determined using the plurality of reference contributions and the known concentrations. The plurality of cell-free DNA fragments analyzed for an F-profile may have a first size with a lower bound greater than 160 bp and an upper bound greater than 230 bp.

Method 2600 may further include comparing the first concentration to a threshold. If the first concentration exceeds the threshold, the method 2600 may include determining a property of the biological sample based on an analysis of cell-free DNA fragments of the biological sample. The plurality of cell-free DNA fragments may be from a first portion of the biological sample. Cell-free DNA fragments of a second portion of the biological sample may be analyzed to determine the property of the biological sample. In some examples, the plurality of cell-free DNA fragments used to determine the first concentration are used to determine the property of the biological sample

### IV. COMBINED SIZE AND END MOTIF ANALYSIS

Techniques can also use both the size and end motif analysis. For example, the independent concentrations from both techniques can be averaged. Alternatively, a multi-parameter regression can be performed, e.g., of a particular size and a particular end motif. In other implementations, a machine learning model can use a feature vector with one or more values for size (e.g., a size profile as described herein, such as in FIGS. 10A-15 and one or more values for a set of end motifs, e.g., as listed in FIGS. 20-21 and table 1. Such a machine learning method provides a unique advantage as it incorporates both the size and end motif differences associated with a particular physiological condition or disease.

### A. Machine Learning Using End Motifs and Size Profile

In some embodiments, machine learning techniques can use size and end motifs. An ML model can estimate cfDNA concentration using amount(s) of fragments having one or more sizes and amount(s) of end motifs having a set of one or more sequence motifs. A feature vector can be generated using the amounts (e.g., relative frequencies) of cfDNA fragments of particular sizes or size ranges and the relative frequencies of end motifs. A machine learning model can then process the feature vector. Same types of machine learning models for size and end motifs can be used for processing both together.

The features for both size and end motifs can be generated in various ways. For example, the two sets of features can be concatenated. Thus, if a size profile has amounts for 100 sizes and 50 end motifs are used, then the feature vector can have a length of 150.

As another example, a matrix of the sizes and end motifs can be generated, so that the number of features is 100×50. For example, for each of M sizes, a set of N relative frequencies for a set of N sequence motifs is determined. The set of N sequence motifs can correspond to the ending sequences of the plurality of cell-free DNA fragments of the size. A relative frequency of a sequence motif can provide a proportion of the plurality of cell-free DNA fragments of a specified size that have an ending sequence corresponding to the sequence motif. In other embodiments, the normalization can be done for a given end motif across different sizes.

In some examples, M can be an integer equal to or greater than, e.g., 10, 20, 30, 40, 50, 60, 70, 80, 90, 100, 200, 300, 400, 500, 600, etc., or any integer there between and N can be an integer equal to or greater than, e.g., 16, 32, 64, 70, 80, 90, 100, 110, 120, 140, 150, 160, 170, 180, 190, 200, 210, 220, 230, 240, 250, 256, or any integer there between. In other examples, M can be less than 10 and/or N can be less than 16. Thus, a feature vector can use the M sets of N relative frequencies of the set of N sequence motifs. The feature vector can include the M sets of N relative frequencies of the set of N sequence motifs in a structured form that can be ingested (input) into and understood by a machine learning model.

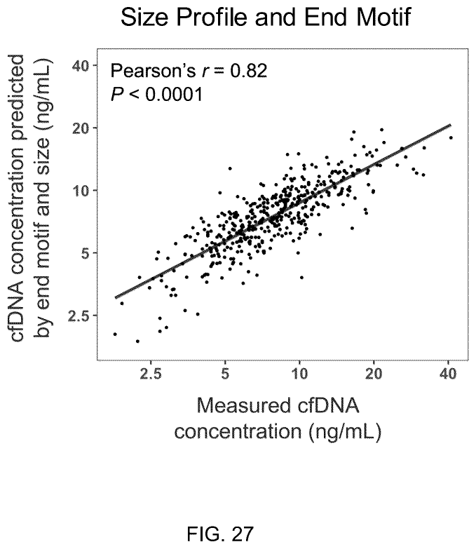

FIG. 27 shows an observed correlation between the cfDNA concentration predicted by SVR and the cfDNA concentration measured by fluorometric quantification in the validation set. Notably, by utilizing the size profile in addition to end motif frequencies, the correlation can be further improved, with a Pearson's r of 0.82 (P<0.0001). These results further validated that the overall cfDNA concentration was associated with characteristic fragmentomic patterns in the cfDNA pool.

In some embodiments, the SVR model can be built by the ‘e1071’ package in R 4.1.2. In the implementation used, the 862 subjects from the target sequencing dataset were randomly grouped as training set (n=431 subjects) and testing set (n=431 subjects) without overlap. Frequencies of 4-mer motifs (256 motifs) and frequencies of cfDNA at different sizes (20˜600 bp; 581 sizes) were used as features to build the model. The prediction target was the cfDNA concentration after logarithmic processing, e.g., values in FIG. 10B or 11B, although raw values without processing can be used such as in FIGS. 10A and 11A. The default radial kernel was used in the SVR model, and the gamma and cost values were tuned by the function ‘tune.svm’. After the model was built, the testing dataset was used to evaluate the predictive accuracy, with 20 subjects from EBV-negative cohort being used as a second testing dataset.

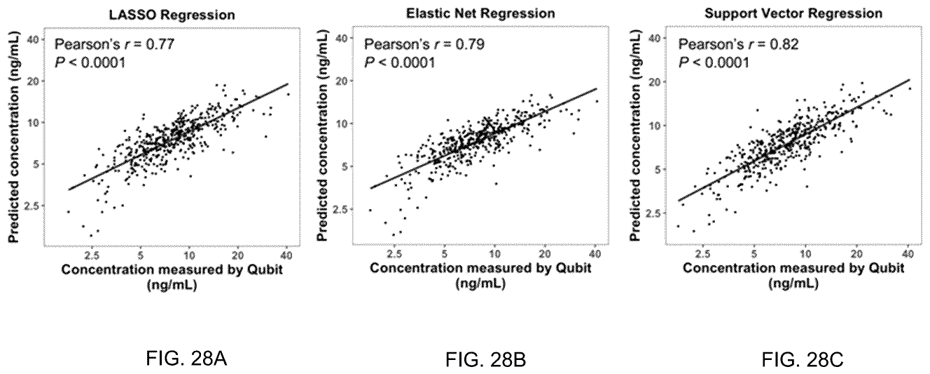

FIG. 28A shows a plot of the correlation between measured cfDNA concentration and cfDNA concentration predicted by end motifs and size using LASSO regression, according to embodiments of the present disclosure. FIG. 28B shows a plot of the correlation between measured cfDNA concentration and cfDNA concentration predicted by end motifs and size using elastic net regression, according to embodiments of the present disclosure. FIG. 28C shows a plot of the correlation between measured cfDNA concentration and cfDNA concentration predicted by end motifs and size using support vector regression, according to embodiments of the present disclosure.

### B. Gradation for F-Profiles for Different Size Ranges

Instead of specific end motifs, a profile of relative of amounts of different end motifs can be used. Such F-profiles are described in more detail below. The percentage contributions of six F-profiles were performed as previously described (Zhou et al. 2023). The percentage contribution of each F-profile in a cfDNA sample can be deduced using a deconvolution procedure, e.g., using non-negative least squares in a deconvolution analysis of the previous established data matrix. We have performed the F-profile analysis using DNA fragments stratified into different size ranges: 20-160 bp, 161-230 bp, and 231-600 bp. However, the F-profile analysis can also be performed independently, without a size analysis. Multiple linear regression was performed on all the significantly correlated F-profiles in each size range. The range 231-600 bp showed an ability to determine cfDNA concentration accurately. Various size ranges can be used, as described herein, e.g., 68 ranges of 10-bp for each, such as for FIG. 13.

### 1. F-Profiles

An F-profile (also referred to as an “end-motif profile”) can correspond to a set of relative frequencies for a set of end motifs, where the sum of the relative frequencies is 100% or 1, depending on the how the relative frequency is defined. For example, if the F-profile was for the 256 4-mer end motifs, the F-profile would have 256 frequency values, which are normalized so that they sum to 100% or 1. Each reference F-profile and sample end-motif profile can have a separate proportion for each K-mer end motif of a set of K-mer end motifs. Accordingly, each reference F-profile of the set of reference F-profiles can specify the proportion of cell-free DNA molecules that end in each K-mer end motif of a set of K-mer end motifs, wherein K is one or two or more.

An F-profile can be chosen arbitrarily or chosen based on a biological process, e.g., correspond to a particular nuclease or other fragmentation process. For example, each reference F-profile can be associated with a type of fragmentation factors. The type of fragmentation factor can identify a particular enzyme (e.g., DNASE1L3, DNASE1), protein (e.g., DFFB), or other biological components or processes that cause fragmentation in cell-free DNA molecules. For example, as was described in section I.C, cfDNA concentration can vary with any amount of DNASE1L3. In some instances, the set of reference F-profiles include 1, 2, 3, 4, 5, 6, 7, 8, 9, 10, 15, 20, 25, 30, 40, 46, 50, or more than 50 F-profiles. For example, the set of reference F-profiles can include six F-profiles I-VI.

In some embodiments, a normalized end motif frequency can be calculated as a ratio of observed and expected frequencies (e.g., for a particular region) and then divided by the sum of all 256 normalized motif frequencies. The total normalized end motif frequency can be equal to 100%. The end motif frequency was termed the normalized end motif frequency.

### 2. Deconvolution

A number of nuclease activities or other fragmentation processes can be assessed simultaneously using deduced relative contributions concerning the different types of cell-free DNA cleavage. For example, relative frequencies of DNA molecules corresponding to 256 end motifs can be determined for a subject with a known disease diagnosis (e.g., HCC). The relative frequencies of DNA molecules can be factorized to a set of “F-profiles” that identify the relationship of ending sequences (e.g., 1-30 bases) of cell-free DNA fragments (also just referred to as DNA fragments) in the sample. The set of F-profiles can then be used in deconvolution of relative frequencies of DNA molecules obtained from another subject to predict cfDNA concentration or fraction of clinically-relevant DNA molecules.

The proportional contributions can be determined by applying deconvolution to the normalized end frequencies. For example, a data matrix M of dimensions W by F can be used, in which: (i) M can represent the normalized end frequencies across 256 end motifs for a given biological sample; (ii) F can represent end frequencies of the reference F-profiles, which may be obtained from murine samples; and (iii) W can represent relative weights corresponding to the proportional contributions of each F-profile.

The F end frequencies can be determined based on the proportions of the cell-free DNA molecules of the set of reference F-profiles. The proportional contributions can be determined by solving for the W relative weights based on using non-negative least square (NNLS) on values from the data matrix M and the reference F-profiles. The proportional contributions determined using deconvolution can be used to determine to a concentration of cfDNA, e.g., total or fractional concentration of clinically-relevant DNA.

In some instances, the set of reference F-profiles are determined using one or more reference samples. The reference samples can be obtained from non-human subjects (e.g., murine samples) whose classification of genetic disorders are known (e.g., WT, DNASE1L3^{-/-}, DNASE1^{-/-}). To determine a reference F-profile of the set of reference profiles, a factorization algorithm (e.g., non-negative matrix factorization (NMF), principal component analysis (PCA)) can be used to decompose the relative frequencies of the cell-free DNA molecules of the reference samples into several F-profiles. For example, reference cell-free DNA samples with different genotypes of DNA nuclease knockouts can be selected. After obtaining the end-motif frequencies of the reference samples, a data matrix (M) can be constructed in a way that each row indicates a cell-free DNA sample (e.g., a total of 93 murine cell-free DNA samples) and each column represents a type of end motif (e.g., a total of 256 4-mer end motifs), thus having the dimension of 93×256. The data matrix can then be subjected to NMF analysis for obtaining two matrices Wand F.

$$M = W F .$$

M is the result of the product of W and F where W was the relative weight for each F-profile in a 93×n matrix, where n corresponded to the number of F-profiles. F represented the F-profiles in an n×256 matrix. W and F were determined by minimizing the objective function below:

$$‖ M - W F ‖ , subject to W \geq 0 and F \geq 0.$$

The proportional contributions of the set of reference F-profiles sum to one, which is the same as 100%.

In some instances, frequencies of 4-mer end motifs of the sample end-motif profile of the subject (e.g., a human subject) and those of reference samples (e.g., murine samples) are normalized by the genomic contexts of their respective genomes. For example, an expected 4-mer end-motif frequency can be used for the normalization step, in which the expected end-motif frequency was determined by simulating 4-mer end motifs from a reference genome using a 4-bp sliding window across each chromosome. The normalized end motif frequency was calculated as a ratio of observed and expected frequencies and then divided by the sum of all 256 normalized motif frequencies. The total normalized end motif frequency can be equal to 100%.

### 3. Contribution of F-Profile Vs CFDNA Concentration

Our group has previously reported on six distinct cleavage patterns via a non-negative matrix factorization (NMF) algorithm, termed “founder” end motif profiles (F-profile), which were linked to different biological processes (Zhou et al. 2023). F-profiles I, II and III represent the contribution of DNASE1L3, DNASE1 and DFFB, respectively. F-profile IV was a cleavage profile with a high C-end preference (e.g., CG-end preference), while F-profile V exhibited a strong G-end preference. Profile VI represents non-specific cleavage patterns, speculated to originate from chemical factors such as oxidative stress. We investigated whether the F-profile contributions varied between subjects of different cfDNA concentrations.

We stratified the DNA fragments into three size ranges for the end motif based deconvolutional analysis, which were 20-160 bp, 161-230 bp and 231-600 bp; selected based on the correlation to cfDNA concentration in FIG. 13. We reasoned that each size range might result from different stages of the fragmentation process of DNA in plasma.

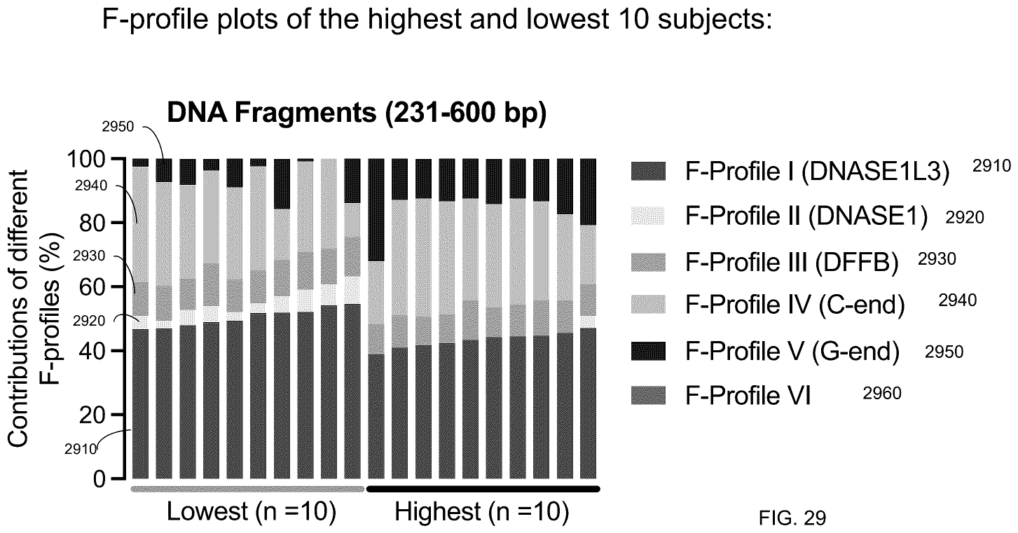

FIG. 29 shows contributions of different F-profiles for the subjects having the highest and lowest cfDNA concentration for DNA fragments within a size range of 231-600 bp. The different colors represent different F-profiles 2910-2960. The vertical height of each color corresponds to an amount of contribution for that F-profile. F-profile I 2910 had the largest contribution, with higher contributions for the samples with the lowest cfDNA contribution relative to the samples with the highest cfDNA contribution. F-profile IV 2940 generally had the next highest contributions. F-profile II 2920 had a very small contribution for the samples with the highest cfDNA contribution, but a more appreciable contribution for the samples with the lowest cfDNA contribution. In contrast, F-profile V 2920 had a small contribution for the samples with the lowest cfDNA contribution, but a larger contribution for the samples with the highest cfDNA contribution. F-profile VI had a negligible contribution.[0242] FIG. 30 shows box and whisker plots of the contributions from five of the F-profiles for DNA fragments within a size range of 231-600 bp. As one can see, the F-profile contributions between subjects with low and highest cfDNA concentrations were markedly different from F-profiles I (DNASEiL3), II (DNASE1), and III (DFFB), and V.

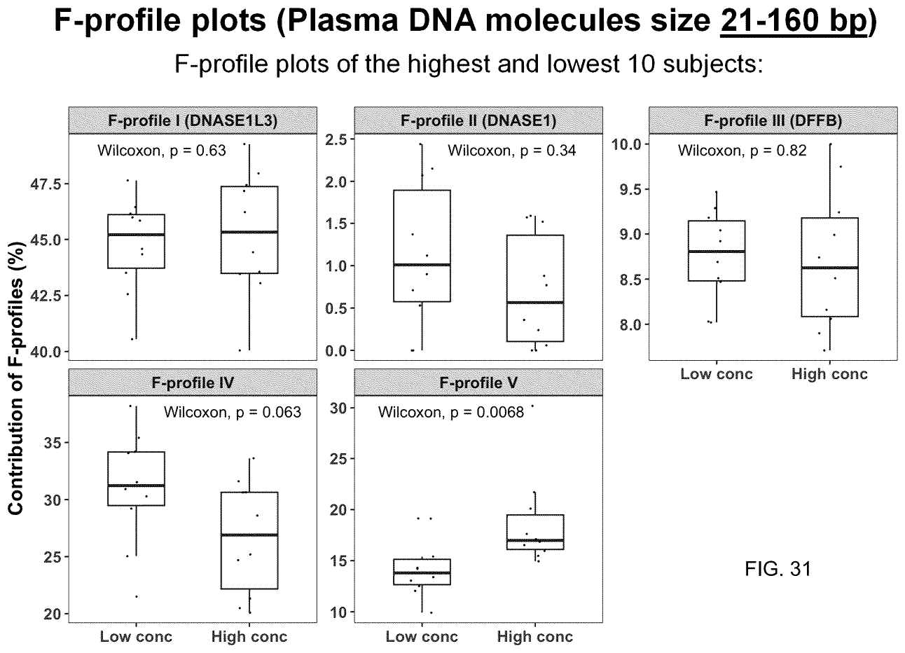

FIG. 31 shows box and whisker plots of the contributions from five of the F-profiles for DNA fragments within a size range of 21-160 bp. The F-profile contributions between subjects with low and highest cfDNA concentrations were less different for this size range compared to 231-600 bp as shown in FIG. 30.

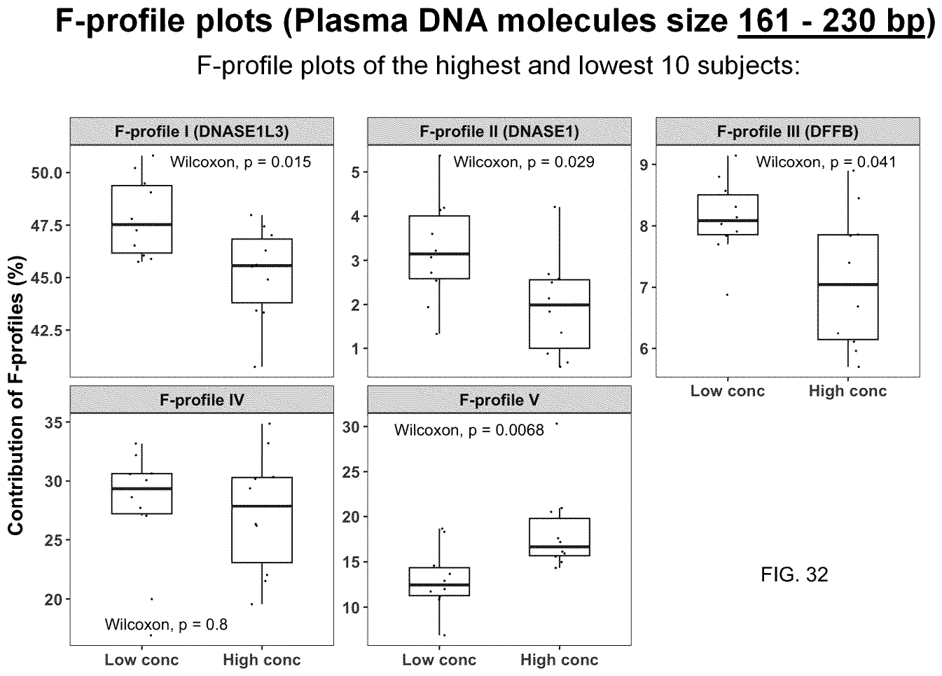

FIG. 32 shows box and whisker plots of the contributions from five of the F-profiles for DNA fragments within a size range of 161-230 bp. The F-profile contributions between subjects with low and highest cfDNA concentrations were also less different for this size range compared to 231-600 bp as shown in FIG. 30.

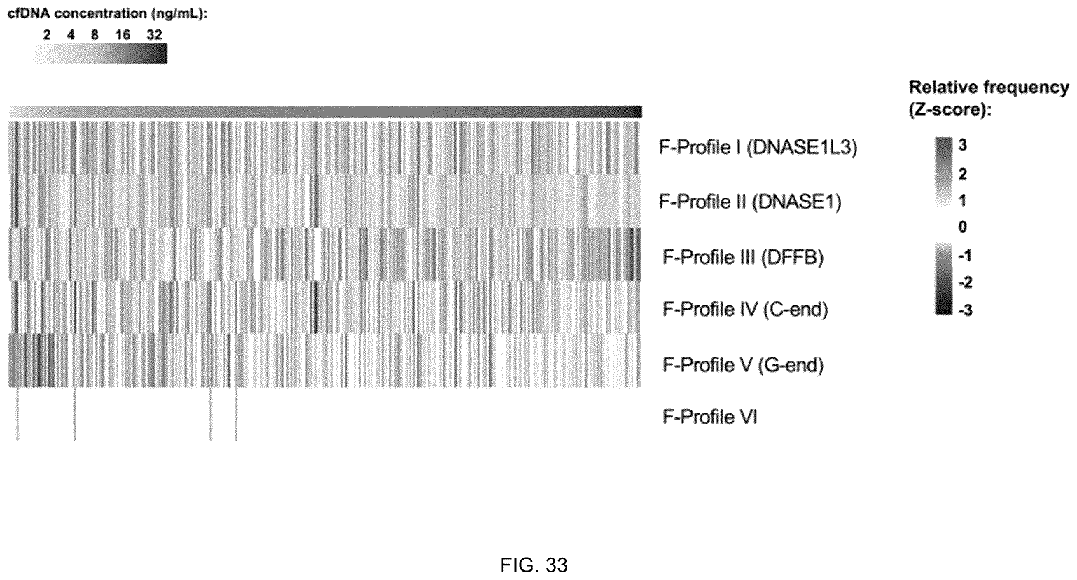

FIG. 33 shows a plot of a heatmap analysis, with each row showing the contribution of each F-profile for DNA fragments within a size range of 231-600 bp across subjects with different cfDNA concentrations, according to embodiments of the present disclosure. The cfDNA concentrations increase from left to right. As one can see, the relative frequency of F-profile I decreases from positive to negative as the cfDNA concentration increases, as does Fi-profile II. The relative frequency of F-profile V increases from negative to positive as the cfDNA concentration increases.

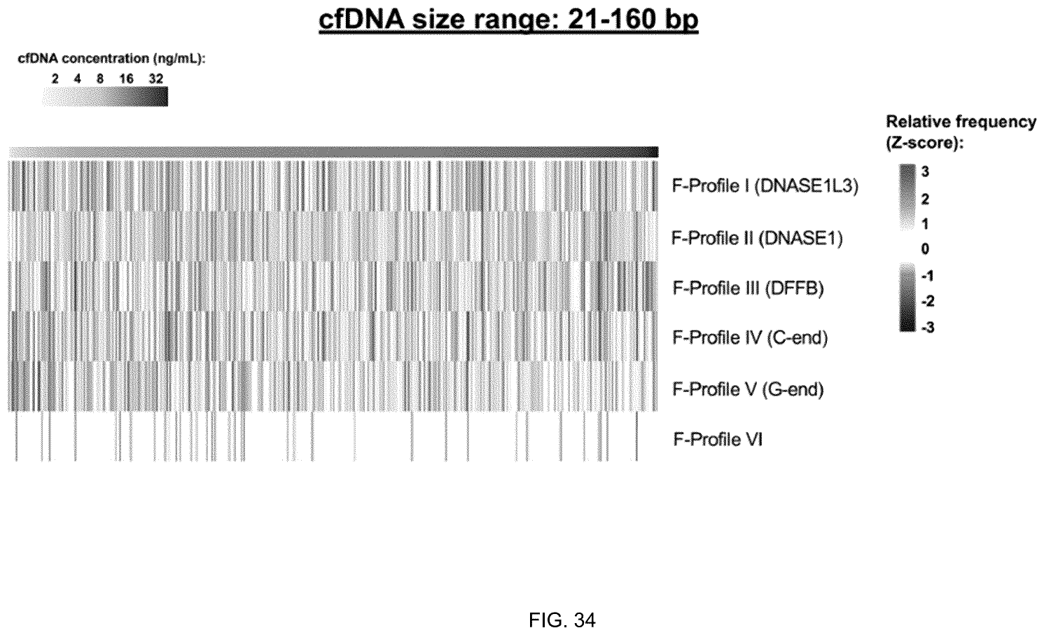

FIG. 34 shows a plot of a heatmap analysis for visualizing F-profile contributions of plasma DNA between subjects of different cfDNA concentrations for the 21-160 bp size range, according to embodiments of the present disclosure. The F-profile contributions between subjects with low and highest cfDNA concentrations were less different for this size range compared to 231-600 bp as shown in FIG. 33.

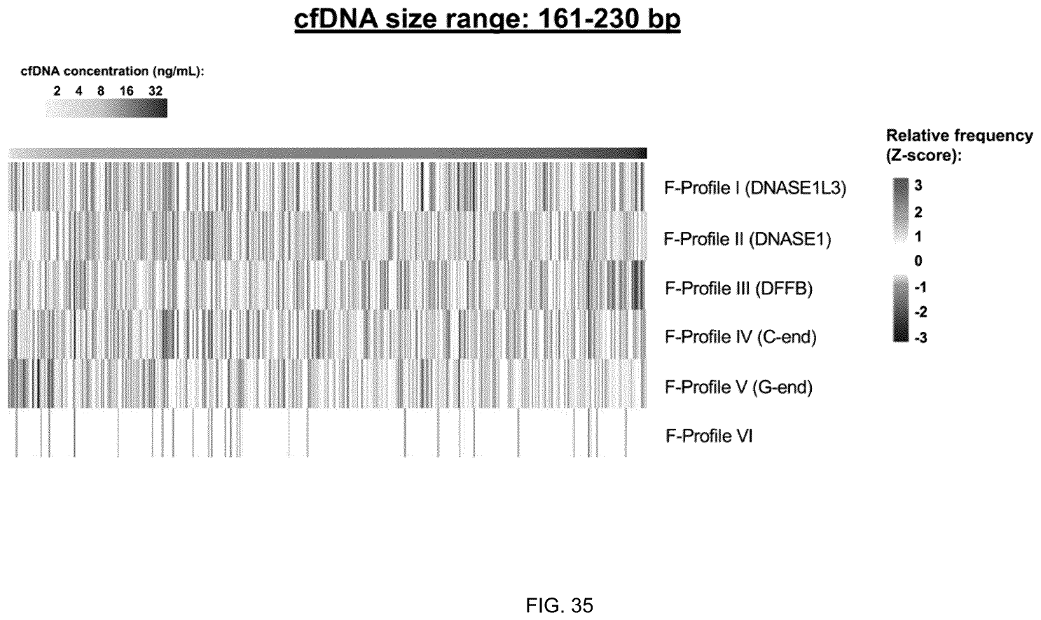

FIG. 35 shows a plot of a heatmap analysis for visualizing F-profile contributions of plasma DNA between subjects of different cfDNA concentrations for the 161-230 bp size range, according to embodiments of the present disclosure. The F-profile contributions between subjects with low and highest cfDNA concentrations were less different for this size range compared to 231-600 bp as shown in FIG. 33.

Accordingly, we visualized the differences in F-profile contributions with different cfDNA concentrations using a heatmap analysis, with Z-score normalization applied to each F-profile. Of the three size ranges, the 231-600 bp size range showed the most distinct variation in F-profile contributions with cfDNA concentration. The contribution of F-profile I (DNASE1L3), II (DNASE1) and III (DFFB) exhibited a decreasing gradation pattern with increased cfDNA concentration. In contrast, F-profile V showed increasing frequencies with cfDNA concentration, which was consistent with the higher frequency of G-end motifs with increasing cfDNA concentration. Overall, the larger plasma DNA fragments with sizes around the di- and tri-nucleosome peaks had a higher frequency of G-end motifs, and decreased end motif frequencies of those from DNA nucleases. No particular gradation pattern was observed amongst all six F-profiles in the 20-160 bp size range. Interestingly, the gradation pattern in F-profiles I, III and V was also observed in the 161-230 bp size range.

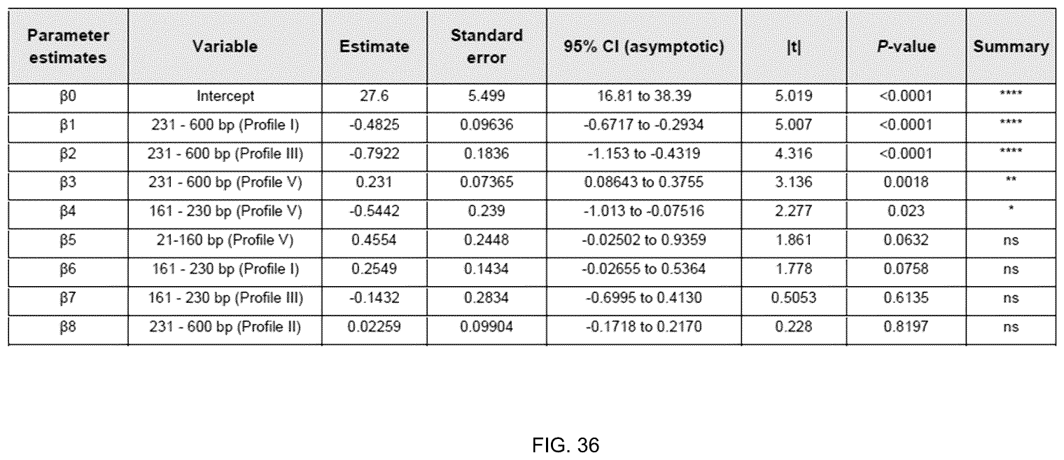

FIG. 36 is a table showing multiple linear regression analysis of size stratified F-profile contributions to cfDNA concentration in the studied cohort, according to embodiments of the present disclosure. A multiple linear regression analysis was performed on all six F-profiles across three size ranges to determine which profiles were significantly associated with the cfDNA concentration. The analysis revealed that F-profiles I (DNASE1L3), III (DFFB) and V (G-ends) of molecules within the 231-600 bp range, and F-profile V within the 161-230 bp range, were significantly associated with the cfDNA concentration. These results provide evidence that DNASE1L3 and DFFB are factors that may regulate the concentration of circulating cfDNA in plasma.

Accordingly, in some embodiments, the method 2600 (and a combination of method 2600 with method 1600) may further include storing a set of reference F-profiles. For each sequence motif of a set of sequence motifs, each reference F-profiles of the set may identify a proportion of cell-free DNA molecules having the sequence motif. Each reference profile of the set may be associated with a type of fragmentation factors. The method 2600 may also include determining a sample end-motif profile by measuring other amounts of cell-free DNA fragments having other sequence motifs corresponding to ending sequence motifs corresponding to ending sequence of the cell-free DNA fragments. The method 2600 may also include determining proportional contributions of the set of reference F-profiles whose proportional aggregation provide the sample end-motif profile. The method 2600 may determine the first concentration of all cell-free DNA in the biological sample using a first proportional contribution of a reference F-profile of the set of reference F-profiles and reference contributions determined from the calibration samples.

### V. TISSUE OF ORIGIN ANALYSIS

An extension of potential applications is whether there is a gradation fragmentomic pattern (e.g., a similar approach of machine learning) for various conditions. We integrated fragmentomic features to look at the fetal and tumor fraction. This would represent a subset of cfDNA concentration that has some clinical relevance. As examples, clinically-relevant DNA can include fetal DNA, tumor DNA, or transplant DNA.

As the total cfDNA concentration of individuals could be predicted using fragment sizes and end motifs, we investigated whether such fragmentomic features could be applied to subsets of cfDNA species. We used circulating tumor DNA in HCC patients and circulating fetal DNA in the plasma of pregnant women as examples, where fragmentomic features was used to predict tumoral and fetal DNA fractions, respectively. Our previous work has demonstrated that fetal and tumor-derived DNA have a shorter modal size of 143 bp (Yu et al. 2014; Jiang et al. 2015; Jiang and Lo 2016). This may be attributed to differences in chromatin packing and methylation density (Sun et al. 2018; Pastor and Kwon 2022). The results below are surprising in that longer cfDNA fragments (as opposed to shorter cfDNA fragments) can be used to provide a fetal fraction of the cfDNA.

Characteristic changes in the end motif profile have also been observed in cancer and pregnancy in general for an entire set of end motifs. However, it surprising that certain end motif(s) could provide an accurate measurement of a fractional concentration of clinically-relevant DNA. Use of a relatively small amount of end motifs (e.g., of varying sequence) can enable low-cost techniques (e.g., PCR-based) to measure such a fractional concentration. As such, an alteration in fetal or tumoral DNA fraction would lead to changes in fragmentomic features of the cfDNA pool. We reasoned that the integrative analysis of the whole spectrum of fragment sizes and end motifs would enable more accurate prediction of fetal or tumoral DNA fraction.

### A. SVR Results

Fragmentomic features, including the size profile and end motif distribution were used to train an SVR model in the prediction of the fractional DNA percentage, which was correlated to the proportional tissue DNA contribution. Correlation was detected between the fetal DNA fraction predicted by the fragmentomic features and SNP-based methods, using 30 pregnant subjects. Correlation was detected between the tumoral fraction predicted by fragmentomic features and by copy number aberration (ichorCNA). Other models than SVR may be used, e.g., as described herein.

As examples, the SVR models can be built using the leave-one-out strategy to predict fetal fraction in pregnant subjects (n=30) and tumor fraction in patients with HCC (n=20). The same fragmentomic profiles (i.e., end motif and size) were used as training features; with the fetal fractions and tumor fractions as target values for building the SVR model. The samples were from previously published sequencing data (Jiang et al. 2020). Raw sequencing data for HCC and pregnancy cases were obtained from a previous study (Jiang et al. 2020), with EGA accession number EGAS00001003409. The predicted fetal and tumor fraction based on fragmentomic features were correlated to the fractions quantified by FetalQuant and ichorCNA, respectively.

### 1. Fetal

We performed a combined analysis of size and end motif analysis and of an independent size analysis to determine a fetal fraction. An independent end motif analysis is provided in a later section.

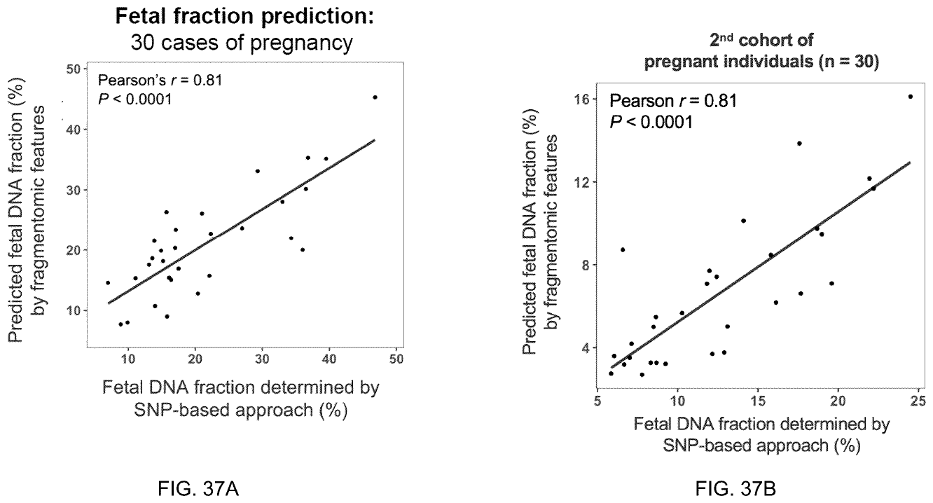

FIGS. 37A-37B show an application of fragmentomic patterns-based deduction of the fractional DNA concentration from fetal cell types. For this combined analysis, the feature vector included both the size profile (each bp from 20-600 bp) and all 256 end motif in the training of the model. Accordingly, the feature vector included 837 features (581 features corresponding to different sizes and 256 features corresponding to the end motifs).

FIG. 37A shows a plot of the correlation between the fetal DNA fraction predicted by the fragmentomic features and SNP-based methods, according to embodiments of the present disclosure. We adopted the leave-one-out procedure and compared the predicted fetal fraction (%) by fragmentomic features to the fetal fraction measured using informative single nucleotide polymorphisms (SNPs). The predicted fetal fraction was strongly positively correlated to SNP-based fetal DNA fraction (Pearson's r=0.81, P<0.0001). The magnitude of correlation exceeded a previous approach that employed the motif diversity score (Spearman r=−0.46, P=0.01) (Jiang et al. 2020).

FIG. 37B shows the correlation between the fetal fraction predicted by fragmentomic features and an SNP-based approach using a test cohort of 30 pregnant subjects. The results are the same, with Pearson's r=0.81, P<0.0001. Accordingly, we further validated our model of fetal fraction prediction by using a second cohort of pregnancy (n=30,) with an SVR model trained with the first cohort (50% training, 50% testing).

We also analyzed the performance of using 231-600 bp DNA fragment size, as well as in combination with end motifs, specifically with 256 4-mer end motifs. In the combined analysis example, the feature vector included 626 features (370 features corresponding to different sizes and 256 features corresponding to end motifs).

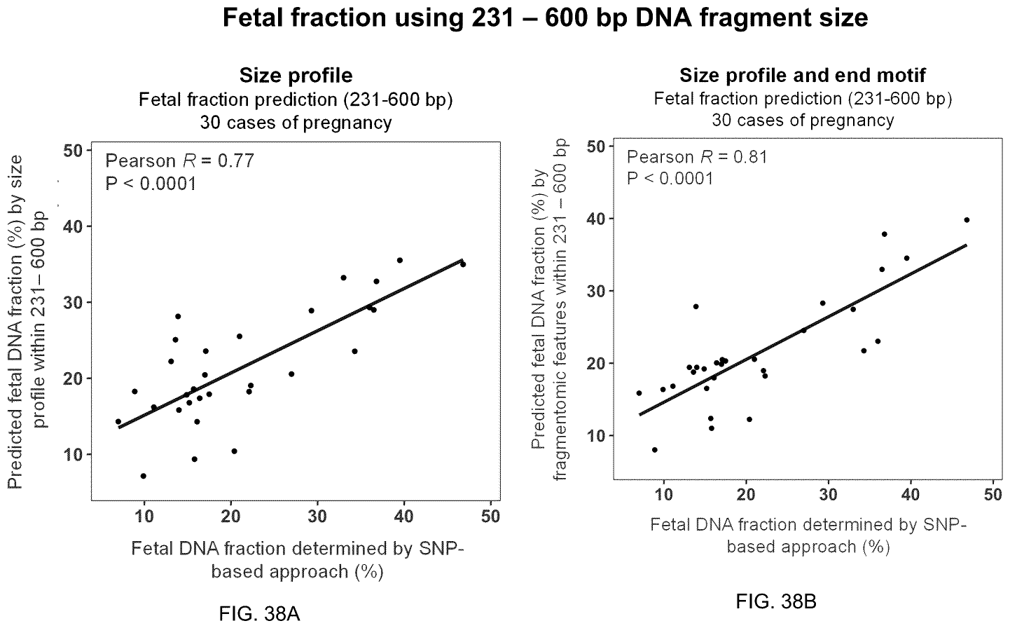

FIGS. 38A-38B show the accuracy in prediction of the fetal fraction using a size profile between 231-600 bp (FIG. 38A) and using the size profile and end motifs (FIG. 38B). This data provides evidence that long fragments is also informative of the fetal fraction. For using sizes only between 231-600 bp, a Pearson r of 0.77 was obtained (FIG. 38A), indicating a surprising result that the long DNA fragments (e.g., a lower cutoff of 231 bp for a lower end of a size range) can be used. The combined technique using size and end motif increased the accuracy slightly to an r=0.81 (FIG. 38B). The results for the combined analysis using 231-600 bp compared to the entire size profile surprisingly shows that equivalent accuracy can be obtained using less information.

Accordingly, we see a strong positive correlation between our predicted fetal DNA fraction and the fetal DNA fraction determined by an SNP-based approach, which is a conventional approach of deducing fetal fraction. Such a technique provides an improvement in not requiring a fetal-specific allele as in the SNP-based approach.

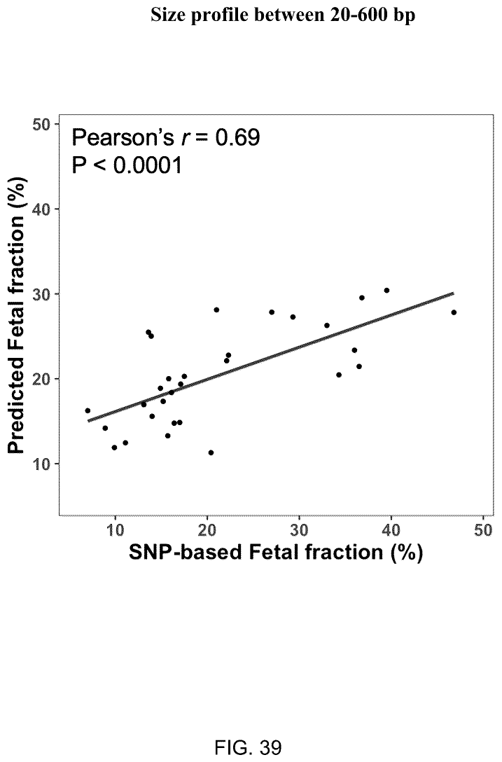

FIG. 39 shows the accuracy in prediction of fetal fraction using a size profile between 20-600 bp. The data provides evidence that using a size profile to predict fetal fraction may be less accurate than using both size and end motifs. And using size profile only with all sizes from 20-600 bp (581 features corresponding to 20-600 bp) resulted in a lower correlation strength (Pearson's r=0.69) compared to using size from only 231-600 bp (FIG. 38A, Pearson's r=0.77) or both size of 231-600 bp and end motif (FIG. 38B, Pearson's r=0.81) from the same dataset.

### 2. Tumor

We performed a combined analysis of size and end motif analysis and of an independent size analysis to determine a tumor fraction. An independent end motif analysis is provided in a later section.

show an application of fragmentomic patterns based deduction of the fractional DNA concentration from fetal cell types. For this combined analysis, the feature vector included both the size profile (each bp from 20-600 bp) and all 256 end motif in the training of the model.

Accordingly, the feature vector included 837 features (581 features corresponding to different sizes and 256 features corresponding to the end motifs).

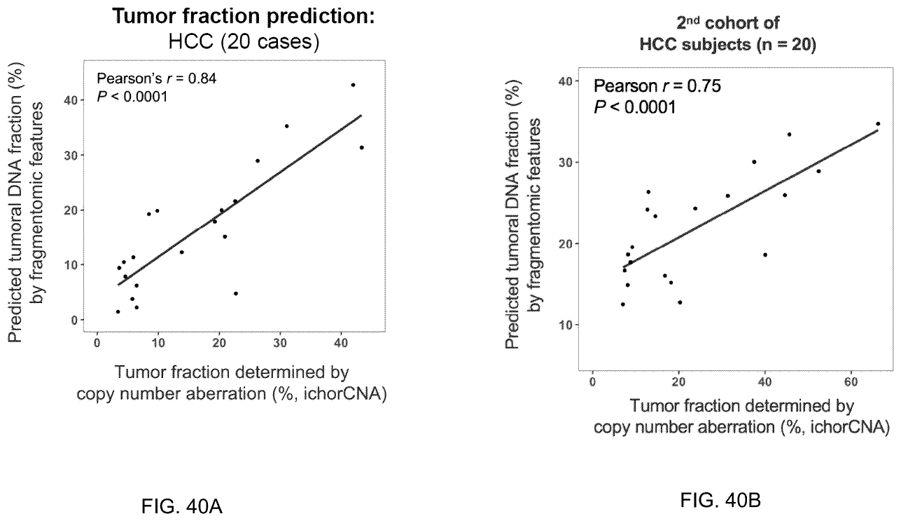

FIG. 40A shows a plot of the correlation between the tumoral fraction predicted by fragmentomic features and by copy number aberration, according to embodiments of the present disclosure. A similar analysis was performed to predict tumor fraction from 20 patients with HCC. The fragmentomic-based predicted tumoral DNA fraction was also positively correlated to the tumor fraction measured by copy number aberration (Pearson's r=0.84, P<0.0001, FIG. 40A). These results also lead to an enhanced correlation of tumor fraction when compared to motif diversity score (Pearson's r=0.65, P=0.0019) (Jiang et al. 2020). We have shown that our fragmentomic feature-based tumor fraction approach also has a strong positive correlation to the conventional approach of using copy number aberrations.

FIG. 40B shows validation of fragmentomic-based deduction of fractional DNA concentration from specific tissue types. FIG. 40B shows the correlation between tumor DNA fraction predicted by fragmentomic features and copy number aberration (ichorCNA), using a test cohort of 20 HCC patients. Our results supported the validity and robustness of tumor fraction prediction (Pearson r=0.75, P<0.0001, FIG. 38B) using fragmentomic features.

We also analyzed the performance of using 231-600 bp DNA fragment size, as well as in combination with end motifs, specifically with 256 4-mer end motifs. In such examples, the feature vector included 626 features (370 features corresponding to different sizes and 256 features corresponding to end motifs).

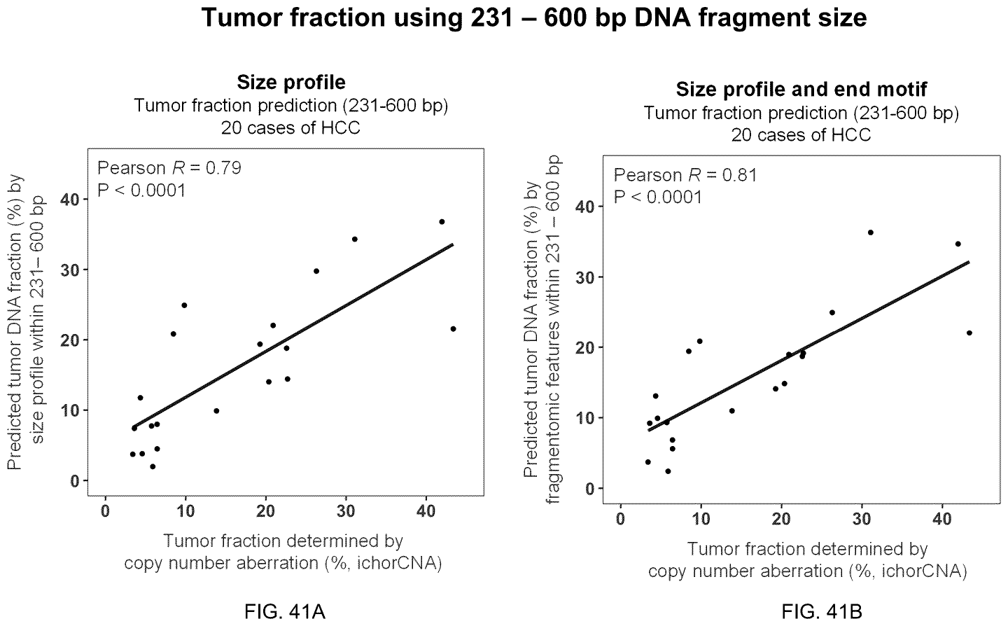

FIGS. 41A-41B show the accuracy in prediction of the tumor fraction using a size profile between 231-600 bp (FIG. 41A) and using the size profile and end motifs (FIG. 41B). For analysis using size profiles between 231-600 bp as shown in FIG. 41A, the feature vector included 370 features corresponding to the different sizes. For combined analysis as shown in FIG. 41B, the feature vector included 626 features (370 features corresponding to different sizes and 256 features corresponding to end motifs). This data provides evidence that long fragments is also informative of the tumor fraction. For using sizes only between 231-600 bp, a Pearson r of 0.79 was obtained (FIG. 41A), indicating a surprising result that the long DNA fragments (e.g., a lower cutoff of 231 bp for a lower end of a size range) can be used. The combined technique using size and end motif increased the accuracy slightly to an r=0.81 (FIG. 41). The results for the combined analysis using 231-600 compared to the entire size profile surprisingly shows that a comparable accuracy can be obtained using less information.

Taken together, the use of fragmentomic patterns not only offered a means to predict the absolute total concentration of cfDNA, but also allowed the deduction of fractional DNA contributions from particular cell types. Such a machine learning method provides a unique advantage as it incorporates both the size and end motif differences associated with a particular physiological condition or disease. These findings open up many further possibilities for analyzing the cfDNA concentration shed from various tissues using cfDNA fragmentomics.

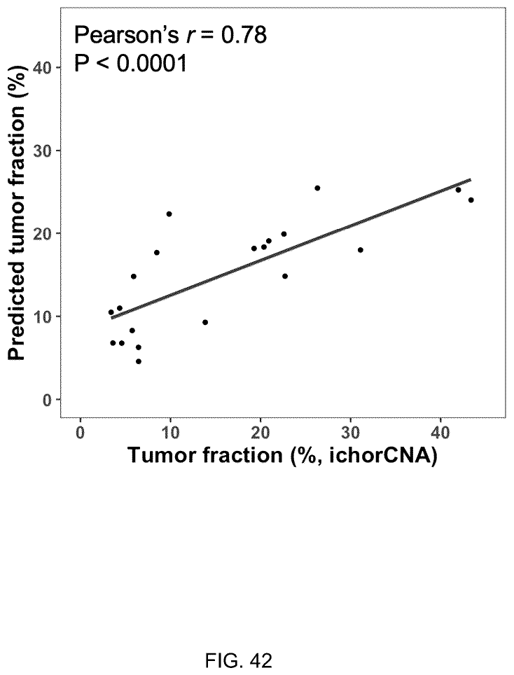

FIG. 42 shows the correlation between tumor DNA fraction predicted by using a size profile between 20-600 bp and copy number aberration (ichorCNA). The data provides evidence that using only size profile to predict tumor fraction may be less accurate than using both size and end motifs. Using size profile only with all sizes from 20-600 bp (581 features corresponding to 20-600 bp) resulted in a slightly lower correlation strength (Pearson's r=0.78) compared to using size from only 231-600 bp (FIG. 41A, Pearson's r=0.79) from the same dataset.

### B. LASSO Results

For further illustration of different machine learning techniques that can be used, we also investigated LASSO regression.

To identify the most informative fragmentomic features in fetal fraction and tumor fraction, LASSO regressions were performed using the first dataset of 30 pregnant women and 20 patients with HCC. In some examples, LASSO regression may performed as a regularization technique to determine coefficients that indicate feature importance.

FIG. 43A is a table 4300 listing features with the highest coefficient value in the prediction of fetal fraction using LASSO regression. LASSO analysis was performed using a leave-one-out method.

FIG. 43B is a table 4310 listing features with the highest coefficient value in the prediction of tumor fraction using LASSO regression.

The use of LASSO regression on the fetal and tumor prediction allowed for the identification of features which are most informative in the model (FIGS. 43A-43B). The most informative features for fetal fraction prediction were the end motifs ACGT, CCGA and CCGT, while that of tumor fraction prediction was the end motif ACGA.

Accordingly, in some embodiments, any of the end motif techniques for determining clinically-relevant DNA (e.g., for fetal tissue), the end motifs ACGT, CCGA, and CCGT can be the only features or some of the features used, e.g., as part of a limited number of end motifs used, such as those selected from Table 3.

Additionally, in some embodiments, any of the end motif techniques for determining clinically-relevant DNA (e.g., for tumor tissue), the end motifs ACGA can be the only features or some of the features used, e.g., as part of a limited number of end motifs used, such as those selected from Table 2.

### C. Use of Different Numbers of End Motifs

As described above with respect to selection of end motifs for more accurate plasma cfDNA prediction predictions, SVR models were trained with ranked order and random selections of end motifs to investigate whether selection of end motifs could result in more accurate fetal fraction and tumor fraction predictions. A first dataset was used from Jiang et al. (2020) Cancer discovery (n=30), and a second dataset was used from new sequencing data (Both datasets used in Malki et al. 2024 Genome Research).

### 1. Fetal

To investigate whether a ranked selection of end motifs could be used to more accurately predict fetal fraction from pregnant women compared to a random selection of end motifs, SVR models for predicting fetal fraction was trained in two ways using cohorts pregnant women from two datasets with 50% set as training and 50% set as testing.

SVR models were trained on a ranked order of end motifs or a random selection of end motifs. The ranked order of end motifs included the most well correlated end motifs to fetal fraction. The most well correlated end motifs to fetal fraction were identified as end motifs with the lowest P-value between the correlation of individual 4-mer end motif frequency and fetal fraction in plasma (See Table 3).

The SVR model was trained with different numbers of end motifs. The ranked order of end motifs included the top 5, 10, 20, 40, 80, 160, or 256 end motifs ordered by P-value of the correlation of each end motif to fetal fraction. The random selection of end motifs included random selections of 5, 10, 20, 40, 80, 160, or 256 end motifs. 100 random selections were performed for each number of end motifs. Samples used for training and testing were kept consistent (i.e., the same n=10 samples from each cohort were used for training, and the remaining n=10 samples from each cohort were used for testing).

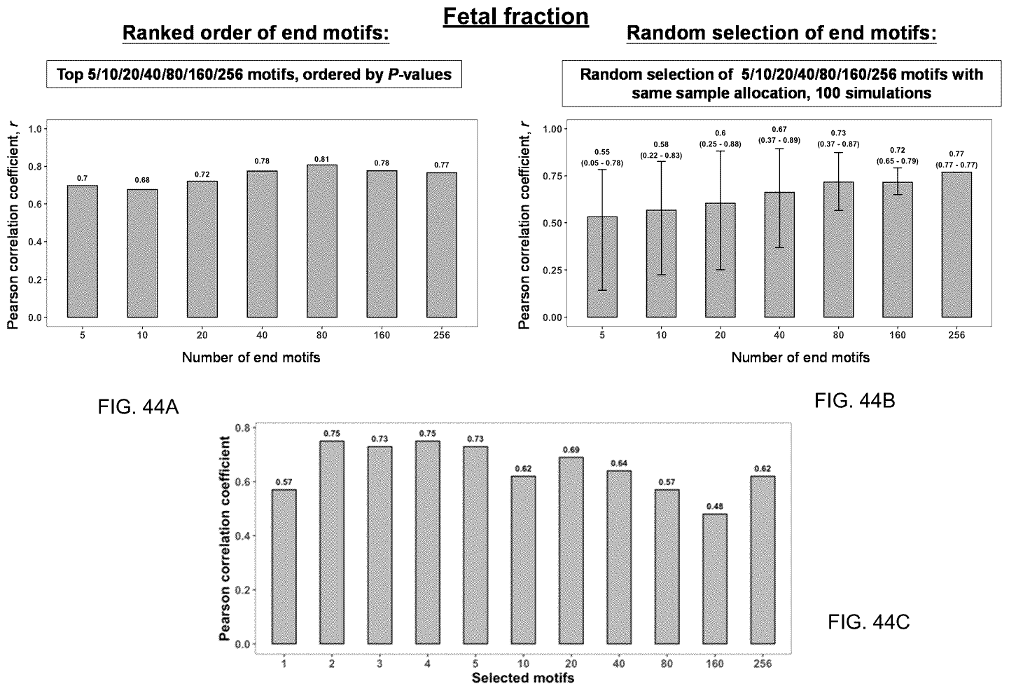

FIGS. 44A-44B show bar plots of correlations between the measured and predicted fetal fraction using different numbers of end motifs. FIG. 44A shows a bar plot of correlations for ranked order of end motifs. The data reveals that a selection of top-ranked end motifs (e.g., 5, 10, 20 motifs) is well-correlated to total cfDNA concentration and fractional cfDNA concentration, including fetal fraction.

The Pearson's r for the top one motif (CGAT) was 0.56 for the testing set, and the top two motifs (CGAT and ACGT) was 0.65 for the testing set; top five was 0.7 for the testing set. In the cohort studied, the top 5 end motif results were in strong correlation, and the addition of more end motifs (Top 6-20) did not significantly increase the accuracy of fetal fraction prediction. As shown, the top 5 end motifs showed roughly equivalent accuracy as the top 20. Thus, a targeted assay can be inexpensive with only 5 end motifs being used. Furthermore, the accuracy of using 80 end motifs was surprisingly higher than using all 256 end motifs, and even the accuracy of the top 40 end motifs was marginally higher than using all 256 end motifs. Thus, more accurate results and a more cost-effective targeted assay can be obtained.

FIG. 44B shows a bar plot of correlations for random selection of end motifs. Each value provided is an average of a 100 different random samplings, with the range of values provided in parentheses. When comparing the results from FIG. 44A, the random results are significantly lower. For example, the average r value for 5 random end motifs was 0.55 compared to 0.7 for the top 5 end motifs.

In some instances, certain random selections may provide higher accuracy (e.g., in certain simulations in the higher range of values provided in parentheses) compared to the ranked order of end motifs. Random combinations may include features that by chance fit the noise of the testing dataset better than selected features used in the ranked order of end motifs. When the number of random combinations is large enough, some combinations may fit better with the specific dataset used due to overfitting. For example, a random selection of 5 end motifs (ACCT, CCTG, AAAC, TCCC, and CCGT) produces a Pearson's r of 0.78 when using the testing set, but a Pearson's r of 0.58 when using the training set. As such, random selections may not perform well in the training set, suggesting an effect of chance in the testing dataset.

We also investigated the performance using sizes of 231-600 bp, instead of 20-600 bp, as were used for FIG. 44A.

FIG. 44C shows a bar plot of a selection of top ranked motifs from DNA of only within 231-600 bp for measuring a fetal fraction. An independent ranking of end motifs were performed for this size range. With the use of end motifs of DNA molecules within the size range of 231-600 bp, we find that the use of few motifs (2-5 motifs) generally provide a better prediction power and correlation for fetal fraction, compared to using all sizes (20-600). Table 4 provided the ranked end motifs used for FIG. 44C.

### 2. Tumor

To investigate whether a ranked selection of end motifs could be used to more accurately predict tumor fraction from patients with HCC compared to a random selection of end motifs, an SVR model for predicting tumor fraction was trained in two ways using cohorts of HCC patients from two datasets with 50% training and 50% testing.

SVR models were trained on a ranked order of end motifs or a random selection of end motifs. The ranked order of end motifs included the most well correlated end motifs to the tumor fraction of DNA determined as end motifs with the lowest P-value between the correlation of individual 4-mer end motif frequency to tumor fraction in plasma cfDNA (See Table 2). For example, the top five motifs were end motifs that were most well correlated to tumor fraction from plasma cfDNA as indicated by having the lowest P-value.

The SVR model was trained with different numbers of end motifs. The ranked order of end motifs included the top 5, 10, 20, 40, 80, 160, or 256 end motifs ordered by P-value of the correlation of each end motif to fetal fraction. The random selection of end motifs included random selections of 5, 10, 20, 40, 80, 160, or 256 end motifs. 100 random selections were performed for each number of end motifs. Samples used for training and testing were kept consistent (i.e., the same n=10 samples from each cohort were used for training, and the remaining n=10 samples from each cohort were used for testing).

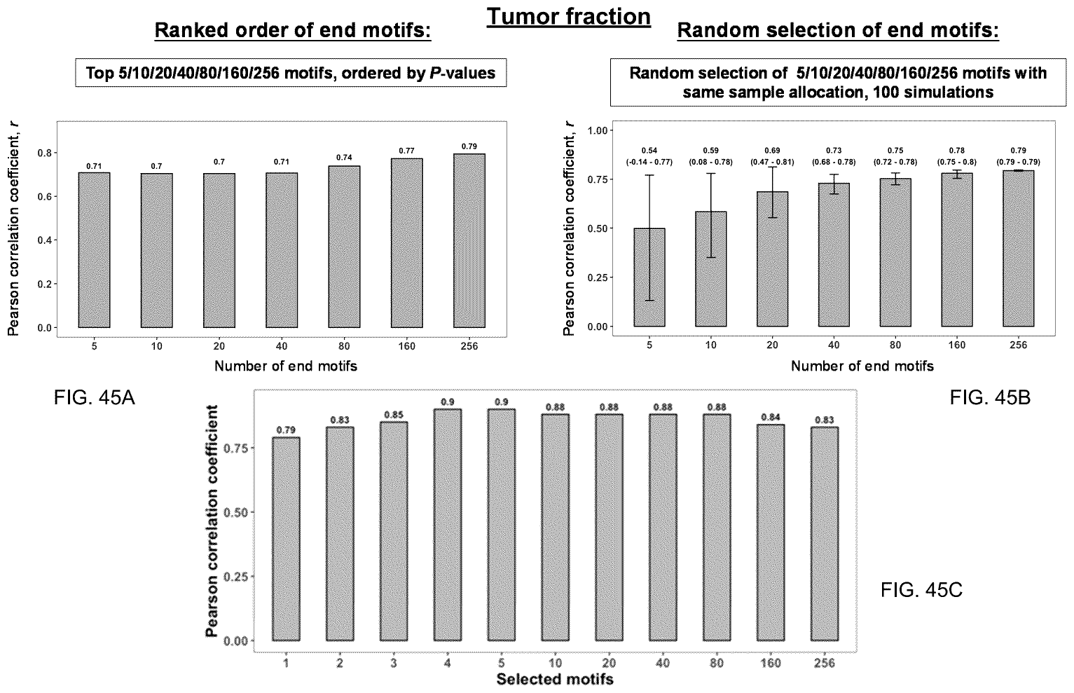

FIGS. 45A-45B show bar plots of correlation between measured and predicted tumor fraction by an SVR model trained using different numbers of end motifs. FIG. 45A shows a bar plot of correlations for ranked order of end motifs. The data reveals that a selection of top-ranked end motifs (e.g., 5, 10, 20 motifs) is well-correlated to total cfDNA concentration and fractional cfDNA concentration, including a tumor fraction.

The Pearson's r for the top one motif (TTAT) was 0.74 for the testing set, while the top two (TTAT and CAGC) was 0.70 for the testing set. In the cohort studied, the top 5 end motif results were in strong correlation, and the addition of more end motifs (Top 6-40) did not significantly increase the accuracy of tumor fraction prediction. As shown, the top 5 end motifs showed roughly equivalent accuracy as the top 20 and top 40. Thus, a targeted assay can be inexpensive with only 5 end motifs being used. Thus, an accurate and cost-effective targeted assay can be obtained. In some embodiments, only the top motif (TTAT) may be used, thereby providing a cost-effective technique (e.g., using a probe-based technique) to determine a tumor fraction.

FIG. 45B shows a bar plot of correlations for random selection of end motifs. Each value provided is an average of a 100 different random samplings, with the range of values provided in parentheses. When comparing the results from FIG. 45B, the random results are significantly lower. For example, the average r value for 5 random end motifs was 0.55 compared to 0.7 for the top 5 end motifs.

Accordingly, the SVR model constructed on ranked selections of end motifs that were most well correlated to tumor fraction generally resulted in greater Pearson's r coefficient values compared to a random selection of motifs. This was particularly observed when a fewer number of end motifs (e.g., the top 5, 10, 20) were used in model training. Furthermore, random selections of end motifs generally resulted in greater variability in correlation strength. For example, random selection of five motifs can result in a Pearson's r within a range of −0.14 to 0.77.

As described above with respect to fetal fraction, in some instances, certain random selections may provide higher accuracy (e.g., in certain simulations in the higher range of values provided in parentheses) compared to the ranked order of end motifs. For example, a random selection of 5 end motifs (GGGC, GGCC, GCCG, AACC, and GCCA) produces a Pearson's r of 0.78 when using the testing set for predicting tumor fraction, but a Pearson's r of 0.58 when using the training set. As such, random selections may not perform well in the training set, suggesting an effect of chance in the testing dataset.

FIG. 45C shows a bar plot of a selection of top ranked motifs from DNA of only within 231-600 bp for measuring a tumor fraction. An independent ranking of end motifs were performed for this size range. With the use of end motifs of DNA molecules within the size range of 231-600 bp, we find that the use of few motifs (1-5 motifs) generally provide a better prediction power and correlation for tumor fraction, compared to using all sizes (20-600). The tumor fraction overall remains quite consistent (or decreased) with the use of more end motifs in this size range, with the top ranked 4 to 5 motifs itself appearing to be quite sufficient in tumor fraction prediction. Table 5 provided the ranked end motifs used for FIG. 44C.

The data for fetal fraction and tumor fraction showed that a selection of end motif (e.g., 1, 2, 3, 4, 5, 10, 20, etc.) is well correlated to fractional cfDNA concentrations, including tumor and fetal fraction. Approaches for targeted sequencing methods can be more easily designed for a smaller dataset of informative motifs to investigate tumor or fetal fraction. Targeted sequencing of smaller numbers of DNA motifs could be a more cost-efficient method of determining fraction DNA concentration. For example, hybridization-based targeted amplification methods and target capture of selected DNA fragments can be used to specifically sequence or capture DNA fragments containing 5′ end motifs of interest. Fewer features of a target design can result in reduced complexity in experimental design, reduced cost, and improved reproducibility, e.g., as further described in section VI.

### D. Method Using Certain End Profiles

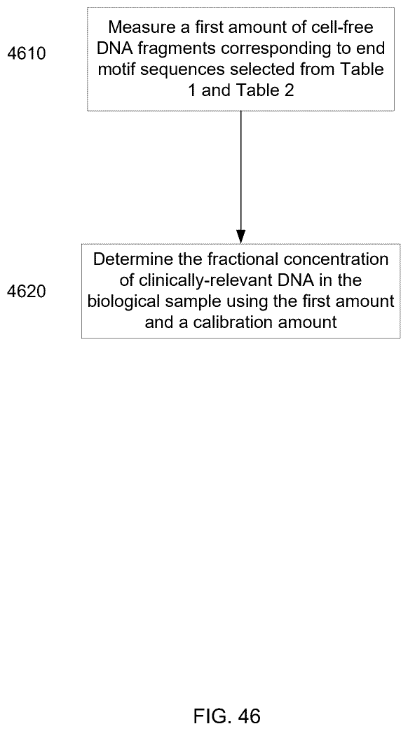

FIG. 46 is a flowchart illustrating a method 4600 for measuring fractional concentration of clinically-relevant DNA in a biological sample. Portions or all steps of method 4600 can be performed by a computer system, including one or more processors. The method 4600 can use a trained ML model that was trained by the computer system or another computer system. The computer system can comprise various devices, e.g., one device that performed the training and another that uses the trained model. Certain steps or procedures performed in method 4600 can be performed in a similar manner as methods 1600 and 2600. As examples, the clinically-relevant DNA can be from a fetus, a tumor, or transplanted tissue.

At block 4610, method 4600 can include measuring a first amount of a plurality of cell-free DNA fragments in the biological sample having a set of one or more end motifs corresponding to ending sequences of the cell-free DNA fragments. The set of one or more sequence motifs can be a set of sequence motifs.

Method 4600 can select the set of one or more sequence motifs from a group consisting of sequence motifs listed in Table 2000 and/or Table 2100 of FIGS. 20-21, as well as any one of Tables 1-3. In some implementations, method 4600 can select the set of one or more sequence motifs from a group consisting of sequence motifs listed in only the top 80 end motifs of Tables 2-5. The measurement can be performed in various ways, e.g., via sequencing or probe-based techniques such as PCR.

Aspects of block 4610 can be performed in a similar manner as block 2610 of method 2600. For example, the set of one or more sequence motifs can be selected from the group consisting of sequence motifs listed in Table 2000. Or, the set of one or more sequence motifs can be selected from the group consisting of sequence motifs listed in Table 2100. Or, the set of one or more sequence motifs can be selected from the group consisting of sequence motifs listed in Table 2 or Table 5 for determining a tumor fraction. Or, the set of one or more sequence motifs can be selected from the group consisting of sequence motifs listed in Table 3 or Table 4 for determining a fetal fraction. Additionally, the top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 or more of these tables can be used. In other examples, the top 1, 2, 3, 4, 5, 6, 7, 8, 9, 10, 11, 12, 13, 14, 15, 16, 17, 18, 19, 20, top 1-5, top 6-10, top 11-15, top 16-20, and top 6-20 ranked end motifs. The top sequence motifs may be the top sequence motifs for a p-value (e.g., adjusted p-value) or a Pearson r value as determined for a training set, a testing set, or both the training set and testing set.

At block 4620, the method 4600 can include determining the fractional concentration of clinically-relevant DNA in the biological sample using the first amount and one or more calibration amounts of cell-free DNA having a set of sequence motifs determined from a calibration sample. Aspects of block 4620 can be performed in a similar manner as block 2620 of method 2600.

For example, the method 4600 may determine the fractional concentration of cell-free DNA in the biological samples using a machine learning model that operates on the first amount and other amounts. The machine learning model may be trained using a training set that includes calibration amounts and known concentrations of the calibration samples. A feature vector derived from the measured cell-free DNA fragments sizes and frequencies may be inputted into the machine learning model. In a particular example, an SVR model may be used to determine the concentration.

Method 4600 may further use the size analysis combined with the end motif analysis, e.g., as described in FIGS. 37A, 37B, 38B, 40A, 40B, and 41B. Accordingly, method 4600 may further include measuring a second amount of the plurality of cell-free DNA fragments having a first size in the biological sample. The first size may have (1) an upper bound less than 231 bp and a lower bound less than 161 bp or (2) a lower bound greater than 160 bp and an upper bound greater than 230 bp. The second amount and one or more calibration amounts determined from one or more additional calibration samples may be used to determine the fractional concentration of clinically-relevant DNA in the biological sample. Each calibration sample may have a known concentration of cell-free DNA. In some examples, the one or more calibration samples are the one or more additional calibration samples.

The method 4600 can further include storing a set of reference F-profiles. For each sequence motif of a set of sequence motifs, each reference F-profile of the set of reference F-profiles may identify a proportion of cell-free DNA molecules having the sequence motif. Each reference F-profile may also be associated with a type of fragmentation factors. The plurality of cell-free DNA may have a first size with a lower bound greater than 160 bp and an upper bound greater than 230 bp.

The method 4600 can further include determining a sample end-motif profile by measuring other amounts of cell-free DNA fragments having other sequence motifs corresponding to ending sequences of the plurality of cell-free DNA fragments. The sample end motif profile can include the first amount and other amounts. Proportional contributions of the set of reference F-profiles whose proportional aggregation provide the sample end motif profile may be determined. The proportional contributions may sum to one. Determining the fractional concentration may use a first proportional contribution of a first reference F-profile of the set of reference F-profile and one or more reference contributions determined from the one or more calibration samples.

Determining the fractional concentration can include comparing the first proportional contribution to the one or mor reference contributions. The one or more reference contributions may be a plurality of reference contributions and comparing the first proportional contribution to the one or more contributions can use a calibration function. The calibration function may be determined using the plurality of reference contributions and the known concentration.

The amount can be an aggregate of all of the sequence motifs in the set of one or more sequence motifs (also referred to as end motifs). In other embodiments, an amount can be determined for each sequence motif in the set, where the set of amounts can be used, e.g., in a machine learning model.

As an example, the method 4600 may further include measuring other amounts of cell-free DNA fragments having other sequence motifs corresponding to ending sequences of the plurality of cell-free DNA fragments. Determining the fraction concentration can include using a machine learning model (e.g., an SVR model) that operates on the first amount and the other amounts. The machine learning model may be trained using a training set that includes the calibration amounts and known concentrations of the calibration samples.

Method 4600 may further include comparing the fractional concentration to a threshold. If the fractional concentration exceeds the threshold, the method 4600 may include determining a property of the biological sample based on an analysis of cell-free DNA fragments of the biological sample. The plurality of cell-free DNA fragments may be from a first portion of the biological sample. Cell-free DNA fragments of a second portion of the biological sample may be analyzed to determine the property of the biological sample. In some examples, the plurality of cell-free DNA fragments used to determine the fractional concentration are used to determine the property of the biological sample.

### E. Method Using Size Profile

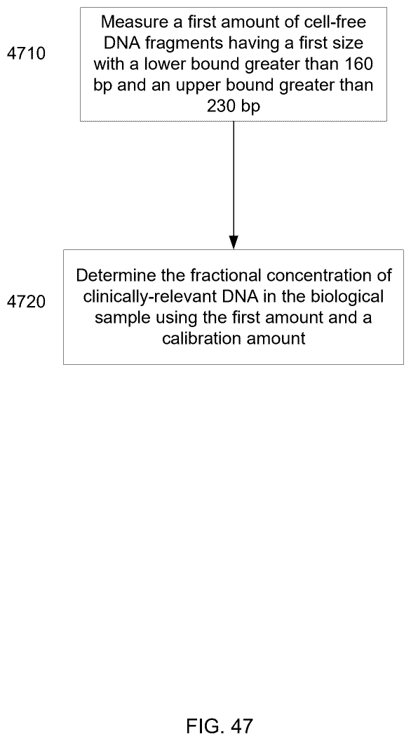

FIG. 47 is a flowchart illustrating a method 4700 for measuring fractional concentration of clinically-relevant DNA in a biological sample. Portions or all steps of method 4700 can be performed by a computer system, including one or more processors. Method 4700 can use a trained ML model that was trained by the computer system or another computer system. The computer system can comprise various devices, e.g., one device that performed the training and another that uses the trained model. Certain steps or procedures performed in method 4700 can be performed in a similar manner as methods 1600, 2600, and 4600. As examples, the clinically-relevant DNA can be from a fetus, a tumor, or transplanted tissue.

At block 4710, the method 4700 can include measuring a first amount of a plurality of cell-free DNA fragments in the biological with a first size having a lower bound greater than 160 bp and an upper bound greater than 230 bp. As examples, the lower bound can be any value between 161 bp to 500 bp. As further examples, the upper bound can be any value between 231 bp to 600 bp. As detailed in the data provided herein, it is surprising that the longer cfDNA fragments can provide accurate results. CFDNA fragments of other sizes can be excluded, e.g., such that the first amount (not counting any normalization) is not determined using amounts of other sizes. Thus, amount(s) of cfDNA fragments below the lower bound are not used, except for via normalization.

Aspects of block 4710 can be performed in a similar manner as block 1610 of method 1600, e.g., the assay for how the measurements is performed. As another example, other amounts of cell-free DNA fragments having other sizes can be used (e.g., for normalization). Determining the fractional concentration of cell-free DNA in the biological sample can include using a machine learning model that (a) operates on the first amount and the other amounts and (b) is trained using a training set that include the plurality of calibration amounts and known concentrations of the plurality of calibration samples. In various embodiments, the other sizes can be within the range 21 bp to 600 bp, the first size and the other sizes can include all sizes within the range 21 bp to 600 bp, and the first size and the other sizes can include all sizes within the range 21 bp to 160 bp and 231 bp to 600 bp.

An amount of a set of one or more sequence motifs can be measured as part of a combined analysis, in a similar manner as for method 1600 and other methods described herein.

At block 4720, the method 4700 can include determining the fractional concentration of clinically-relevant DNA in the biological sample using the first amount and one or more calibration amounts of cell-free DNA having a set of sequence motifs determined from a calibration sample.

In some examples, the method 4700 may determine the fractional concentration of cell-free DNA in the biological samples using a machine learning model that operates on the first amount and other amounts. The machine learning model may be trained using a training set that includes calibration amounts and known concentrations of the calibration samples. A feature vector derived from the measured cell-free DNA fragments sizes and frequencies may be inputted into the machine learning model. In a particular example, an SVR model may be used to determine the concentration.

As another example, determining the fractional concentration can include comparing the first amount to the one or more calibration amounts. When the one or more calibration amounts are a plurality of calibration amounts, comparing the first amount to the one or more calibration amounts uses a calibration function determined using the plurality of calibration amounts and the known concentrations.

Method 4700 may further include comparing the fractional concentration to a threshold. If the fractional concentration exceeds the threshold, the method 4700 may include determining a property of the biological sample based on an analysis of cell-free DNA fragments of the biological sample. The plurality of cell-free DNA fragments may be from a first portion of the biological sample. Cell-free DNA fragments of a second portion of the biological sample may be analyzed to determine the property of the biological sample. In some examples, the plurality of cell-free DNA fragments used to determine the fractional concentration are used to determine the property of the biological sample.

### VI. TARGETED APPROACH

As described above, some embodiments can use fewer than all end motifs for a particular k-mer. Using fewer end motifs can enable a more cost effective targeted approach.

Some assay designs are provided below. For example, 5 to 40 end motifs can be targeted, including the top 1-5, 1-10, 1-20, 1-40, 6-20, and 6-40.

Regarding a targeted approach for specific end motifs, several examples are provided below, e.g., as described in U.S. Pat. Pub No.: US 2023/0374601 and U.S. Pat. Pub No. US 2025/0171858.

### A. Target Based Methods for Selected 5′ End Motifs:

These example methods can specifically sequence or capture DNA fragments containing 5′ end motifs of interest. Several example methods are described for this targeting.

A first approach includes hybridization-based targeted amplification methods, whether specific primer designs can selectively bind DNA fragments containing 5′ end motifs of interest. Primers can contain complementary sequences to common adaptors, with additional nucleotides that bind to the end portion of DNA fragments. Primer extension during PCR would selectively amplify these DNA fragments of interest. Further details are provided U.S. Pat. Pub No.: US 2023/0374601. An alternative design would be to use this hybridization-based method in single stranded library preparations. The splint adaptor design can contain DNA fragments complementary to the end motifs of interest. Only selected end motifs of interest would successfully ligate to DNA adaptors and be amplified during subsequent library preparation and PCR steps.

Another approach includes target capture of selected DNA fragments. Using a similar concept to the primer extension method, another approach would be to design oligos to specifically bind to DNA fragments with specific 5′ end motifs. The oligos can be conjugated with selected chemical moieties (e.g., biotin), which can be targeted by beads-based approaches.

The oligos will contain complementary sequences to the common adaptor, with additional nucleotides binding to selected end motifs. Captured DNA fragments can contain the end motif of interest and be amplified by PCR for sequencing.

### B. Selection of 5′ End Motifs:

To make use of targeted capturing systems to quantify the total cell-free DNA concentration or fractional DNA concentration including the tumor or fetal fraction, embodiments can determine a ‘motif ratio’, e.g., to normalize end motif frequencies in way that is informative of the absolute of fractional DNA concentration.

One approach can select the most (e.g., top N) significantly positively correlated and negatively correlated end motifs to the concentration of cell-free DNA of interest. Taking tumor fraction as an example, the motif frequencies of significant positively correlated end motifs should be increased with higher tumor fraction, while negatively correlated end motifs should be relatively decreased. As such a ratio of motif frequencies of ‘positively correlated motifs/negatively correlated motifs’ should be strongly positively correlated to tumor fraction. Using a targeted approach, a sequencing read count of selected end motifs of interest may accurately inform tumor fraction, without the interference of non-informative motifs in the cfDNA pool. A direct read-out of tumor fraction can be provided. This type of assay requires initial training set of known tumor fraction prior to testing new samples.

Such an approach would allow a quantitative PCR (qPCR) readout. Probes for qPCR can be designed to specifically bind to the 5′ end motifs of interest, and subsequent extension of DNA would allow for accurate quantification of DNA fragments containing specific end motifs. Using the concept of motif ratios described previously, probe oligos targeting positively correlated end motifs can carry one type of fluorescent signal, while probes targeting negatively correlated end motifs with another fluorescent signal. The read-out of both fluorescent signals could serve as a proxy to motif ratio. This may provide a cheaper, non-sequencing-based method of determining cfDNA quantities. A similar design was previously proposed in FIG. 4 of FRAGMA patent (US 2023/0374601 A1).

### VII. EXAMPLE USE CASES

After determining a concentration of all cfDNA or a fractional concentration of clinically-relevant DNA, various embodiments can analyze the cfDNA fragments, e.g., as part of an assay on the sample or the subject. For instance, if the measured concentration is greater than a threshold, then the sample can be deemed valid for further analysis. The cfDNA fragments used to determine the concentration or a different set of cfDNA fragments (e.g., from a second portion of the sample) can be subjected to an assay (e.g., sequencing or probe-based techniques) or data from such an assay can be received and analyzed. Examples assays, example markers (e.g., other than size or end motif), and example results are provided below.

### A. Example Assays

Various techniques can be used for such analysis in any of the methods described in the present disclosure. For example, the analysis can be performed using sequencing, such as massively parallel sequencing, targeted sequencing, and single molecule sequencing (e.g., using a nanopore or using real-time single molecule sequencing (e.g., from Pacific Biosciences)). Example PCR techniques include real-time PCR and digital PCR (e.g., droplet digital PCR). The analysis can include the physical steps of performing such assays and receiving of the measurement data obtained from such assays, or may just include receiving the measurement data.

Analyzing a cell-free DNA molecule can include determining a genomic position in a reference genome corresponding to at least one end of the cell-free DNA molecule. For example, one or more sequence reads of a DNA molecule (e.g., paired reads at the ends or a read for the entire molecule) can be aligned to the reference genome using any of various alignments techniques as will be appreciated by the skilled person. The alignment can be to some or all of the reference genome. As another example, probe-based techniques can identify a DNA molecule as being from a particular position, e.g., by emitting a particular color for a particular probe that corresponds to a particular genomic position. The position determination can be to some or all of the reference genome, e.g., if only part of the genome is being analyzed. As examples, the amount of the genome analyzed can be greater than 0.01%, 0.1%, 1%, 5%, 10%, or 50%. Such an analysis may be performed for other methods described herein.

As with other methods described herein, analyzing the plurality of cell-free DNA molecules can includes measuring a size of the cell-free DNA molecule. The measurement can be performed in various ways, e.g., using physical separation (such as electrophoresis) and/or sequencing (such as whole molecule sequencing or alignment using paired-end reads).

### B. Example Markers

Various markers can be identified through the assays described above, such as copy numbers, sizes, end motifs, jagged ends, sequence variations (mutations), methylation changes, nucleosome signal patterns, preferred ending positions, and nucleosome footprints.

For example, we can detect copy number aberrations from a cell-free DNA sample by counting analysis. The aberration of a region can be determined by counting an amount of DNA fragments (molecules) that are derived from the region. As examples, the amount can be a number of DNA fragments, a number of bases to which a DNA fragment overlapped, or other measure of DNA fragments in a region. The amount of DNA fragments for the region can be determined by sequencing the DNA fragments to obtain sequence reads and aligning the sequence reads to a reference genome. In one embodiment, the amount of sequence reads for the region can be compared to the amount of sequence reads for another region so as to determine overrepresentation (amplification) or underrepresentation (deletion). In another embodiment, the amount of sequence reads can be determined for one haplotype and compared to the amount of sequence reads for another haplotype. Further details of counting analysis to identify aberrant regions is described in U.S. Patent Publication No. 2009/0029377 entitled “Diagnosing fetal chromosomal aneuploidy using massively parallel genomic sequencing” by Lo et al. filed on Jul. 23, 2028; and U.S. Patent Publication No. 2016/0201142 entitled “Using Size and Number Aberrations in Plasma DNA For Detecting Cancer” by Lo et al, filed on Jan. 12, 2016, the disclosures of which are incorporated by reference in their entirety for all purposes.

We can also use a size-based analysis to perform a prenatal diagnosis of a sequence imbalance (e.g. a fetal chromosomal aneuploidy) in a biological sample obtained from a pregnant female subject. For example, a size distribution of fragments of nucleic acid molecules for an at-risk chromosome can be used to determine a fetal chromosomal aneuploidy. The size-based analysis can be also used for a cancer diagnosis. Some embodiments can also detect other sequence imbalances, such as a sequence imbalance in the biological sample (containing mother and fetal DNA, or containing body and cancer DNA), where the imbalance is relative to a genotype, mutation status, or haplotype of the mother. Such an imbalance can be determined via a size distribution of fragments (nucleic acid molecules) corresponding to a particular sequence relative to a size distribution to be expected if the sample were purely from the mother, and not from the fetus and mother, or from a healthy subject. A shift (e.g. to a smaller size distribution) can signify an imbalance in certain circumstances. Details of size-based analysis are described in U.S. Patent Publication No. 2011/0276277 entitled “Size-Based Genomic Analysis” by Lo et al. filed Nov. 5, 2010 (focused on fetal DNA analysis), U.S. Patent Publication No. 2013/0237431 entitled “Size-Based Genomic Analysis of Fetal DNA Fraction in Maternal Plasma” by Lo et al. filed Mar. 7, 2013 (focused on fetal or cancer DNA analysis), and U.S. Patent Publication No. 2019/0130065 entitled “Using Nucleic Acid Size Range For Noninvasive Prenatal Testing And Cancer Detection” by Lo et al. filed Nov. 1, 2018 (focused on cancer DNA analysis), the contents of which are incorporated herein by reference for all purposes.

The present disclosure also provides techniques for measuring quantities (e.g., relative frequencies) of sequence end motifs of cell-free DNA fragments in a biological sample from a subject for measuring a property of the sample (e.g., fractional concentration of clinically relevant DNA) and/or determining a condition of the subject based on such measurements. An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments. Details of end motif analysis are described in U.S. Patent Publication No. 2020/0199656 entitled “Cell-Free DNA End Characteristics” by Lo et al. filed Dec. 19, 2019, the contents of which are incorporated herein by reference for all purposes.

The present disclosure further provides techniques for jagged end analysis. Double-stranded cell-free DNA fragments may often have two strands that are not exactly complementary to each other. One strand may extend beyond the other strand, creating an overhang. These overhangs are often repaired to form blunt ends in analysis. However, the “jagged ends” created by these overhangs may be useful in analyzing biological samples. As an example, jagged ends in cell-free DNA from a urine sample may be used to diagnose or detect a condition noninvasively and accurately. The degree of jagged ends, which may be the quantity or the length of jagged ends, in a sample may reflect the level of a condition in an individual. For example, the degree of jagged ends may be related to a disease (e.g., cancer), a disorder, a pregnancy-related condition, or a transplant condition. Details of jagged end analysis are described in U.S. Patent Publication No. 2020/0056245 entitled “Cell-Free DNA Damage Analysis and Its Clinical Applications” by Lo et al., filed Jul. 23, 2019, and U.S. Patent Publication No. 2022/0177971-A1 entitled “Methods Using Characteristics of Urinary and Other DNA”, filed Dec. 7, 2021, the contents of which are incorporated herein by reference for all purposes.

The present disclosure further provides techniques for sequence variation (mutation) analysis. Details of sequence variation (mutation) analysis are described in U.S. Patent Publication No. 2014/0100121 entitled “A Method of Measuring a Fractional Concentration Of Tumor DNA”, filed Mar. 13, 2013, and U.S. Patent Publication No. 2017/0073774-A1 entitled “Detecting Mutations For Cancer Screening”, filed Nov. 28, 2016, the contents of which are incorporated herein by reference for all purposes.

The present disclosure further provides techniques for methylation analysis. Details of methylation analysis are described in WO 2014/043763, entitled “Non-Invasive Determination of Methylome of Fetus Or Tumor From Plasma”, filed Sep. 20, 2013, the contents of which are incorporated herein by reference for all purposes.

The present disclosure further provides techniques for nucleosome signal pattern analysis. Details of nucleosome signal pattern analysis are described in U.S. patent application Ser. No. 18/883,637, entitled “Uses of Cell-Free DNA Fragmentation Patterns Associated With Epigenetic Modifications”, filed Sep. 12, 2024, the contents of which are incorporated herein by reference for all purposes.

### C. Example Results

The detection of these molecular markers described above can be useful for the screening, detection, monitoring, management, and prognostication of cancer patients, or patients with other diseases or disorders. The exemplary output results include, but not limited to, level of cancer, aneuploidy (copy number) for tumor or fetal, and fraction of clinically-relevant DNA (e.g., tumor, fetal, or transplant).

For example, the assays described herein can be used to detect fetal inheritance, as detailed in U.S. Patent Publication No. 2011/0105353, entitled “Fetal Genomic Analysis from A Maternal Biological Sample, filed Nov. 5, 2010, and U.S. Patent Publication No. 2017/0029900 entitled “Methylation Pattern Analysis Of Haplotypes In Tissues In A DNA Mixture”, filed Jul. 20, 2016 (focused on methylation pattern analysis), the contents of which are incorporated herein by reference for all purposes.

We can also use the assays described herein to measure levels of a cancer, as detailed in U.S. Patent Publication No. 2014/0100121, entitled “A Method Of Measuring A Fractional Concentration Of Tumor DNA”, filed Mar. 13, 2013, and PCT Patent Publication No. WO 2014/043763 entitled “Non-Invasive Determination Of Methylome Of Fetus Or Tumor From Plasma”, filed Sep. 20, 2013, the contents of which are incorporated herein by reference for all purposes.

We can also use the assays described herein to measure fractional concentration, as detailed in U.S. Patent Publication No. 2013/0237431 entitled “Size-Based Analysis Of Fetal Or Tumor DNA Fraction In Plasma”, filed Mar. 7, 2013, and US Patent Publication No. 2020/0199656 entitled “Cell-Free DNA End Characteristics”, filed Dec. 19, 2019, the contents of which are incorporated herein by reference for all purposes.

We can further use the assays described herein to measure copy number (sequence imbalance), as detailed in U.S. Patent Publication No. 2009/0029377 entitled “Diagnosing Fetal Chromosomal Aneuploidy Using Massively Parallel Genomic Sequencing”, filed Jul. 23, 2008, the contents of which are incorporated herein by reference for all purposes

### VIII. TREATMENTS

### A. Further Screening Modalities

Based on any classification, e.g., regarding a pathology or fractional concentration of clinically-relevant DNA, the subject can be referred for additional screening modalities, e.g. using chest X ray, ultrasound, computed tomography, magnetic resonance imaging, or positron emission tomography. Such screening may be performed for cancer.

### B. Treatment Selection

Various embodiments of the present disclosure can accurately predict disease relapse, occurrence, and/or severity thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Various embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### IX. EXAMPLE SYSTEMS

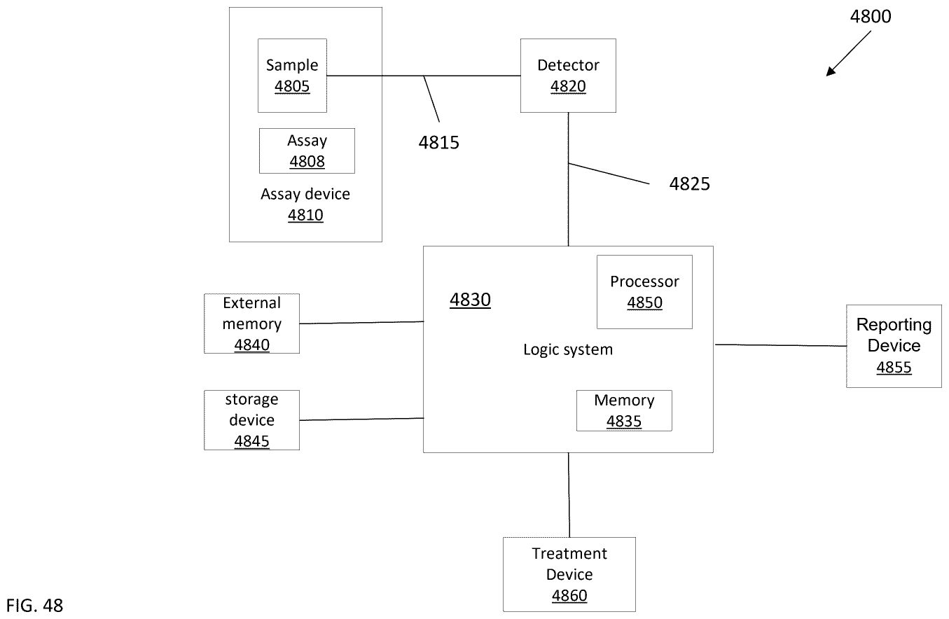

FIG. 48 illustrates a measurement system 4800 according to an embodiment of the present disclosure. The system as shown includes a sample 4805, such as cell-free nucleic acid molecules (e.g., DNA and/or RNA) within an assay device 4810, where an assay 4808 can be performed on sample 4805. For example, sample 4805 can be contacted with reagents of assay 3908 to provide a signal (e.g., an intensity signal) of a physical characteristic 4815 (e.g., sequence information of a cell-free nucleic acid molecule). An example of an assay device can be a flow cell that includes probes and/or primers of an assay or a tube through which a droplet moves (with the droplet including the assay). Physical characteristic 4815 (e.g., a fluorescence intensity, a voltage, or a current), from the sample is detected by detector 4820. Detector 4820 can take a measurement at intervals (e.g., periodic intervals) to obtain data points that make up a data signal. In one embodiment, an analog-to-digital converter converts an analog signal from the detector into digital form at a plurality of times.

Assay device 4810 and detector 4820 can form an assay system, e.g., a PCR system or a sequencing system that performs sequencing according to embodiments described herein. A data signal 4825 is sent from detector 4820 to logic system 4830. As an example, data signal 4825 can be used to determine sequences and/or locations in a reference genome of nucleic acid molecules (e.g., DNA and/or RNA). Data signal 4825 can include various measurements made at a same time, e.g., different colors of fluorescent dyes or different electrical signals for different molecule of sample 4805, and thus data signal 4825 can correspond to multiple signals. Data signal 4825 may be stored in a local memory 4835, an external memory 4840, or a storage device 4845. The assay system can be comprised of multiple assay devices and detectors.

Logic system 4830 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 4830 and the other components may be part of a stand-alone or network connected computer system, or they may be directly attached to or incorporated in a device (e.g., a sequencing device) that includes detector 4820 and/or assay device 4810. Logic system 4830 may also include software that executes in a processor 4850. Logic system 4830 may include a computer readable medium storing instructions for controlling measurement system 4800 to perform any of the methods described herein. For example, logic system 4830 can provide commands to a system that includes assay device 4810 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 4800 may also include a treatment device 4860, which can provide a treatment to the subject. Treatment device 4860 can determine a treatment and/or be used to perform a treatment. Examples of such treatment can include surgery, radiation therapy, chemotherapy, immunotherapy, targeted therapy, hormone therapy, and stem cell transplant.

Logic system 4830 may be connected to treatment device 4860, e.g., to provide results of a method described herein. The treatment device may receive inputs from other devices, such as an imaging device and user inputs (e.g., to control the treatment, such as controls over a robotic system).

Measurement system 4800 may also include a reporting device 4855, which can present results of any of the methods describe herein, e.g., as determined using the measurement system. Reporting device 4855 can be in communication with a reporting module within logic system 4830 that can aggregate, format, and send a report to reporting device 4855. The reporting module can present information determined using any of the method described herein. The information can be presented by reporting device 4855 in any format that can be recognized and interpreted by a user of the measurement system 4800. For example, the information can be presented by reporting device 4855 in a displayed, printed, or transmitted format, or any combination thereof.

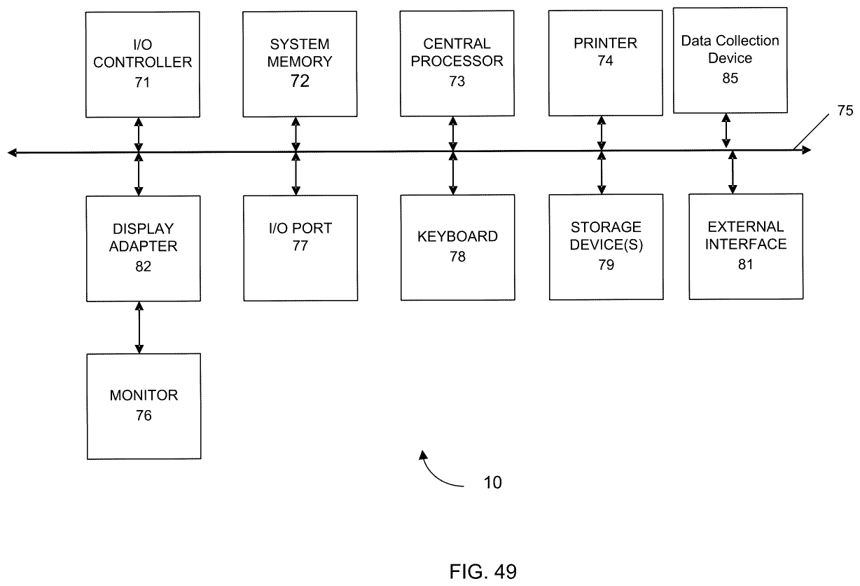

Any of the computer systems mentioned herein may utilize any suitable number of subsystems. Examples of such subsystems are shown in FIG. 49 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 49 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network.

In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. As examples, a time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art.

Where a conflict exists between the instant application and a reference provided herein, the instant application shall dominate.

### X. References

Alborelli I, Generali D, Jermann P, Cappelletti M R, Ferrero G, Scaggiante B, Bortul M, Zanconati F, Nicolet S, Haegele J et al. 2019. Cell-free DNA analysis in healthy individuals by next-generation sequencing: a proof of concept and technical validation study. Cell Death Dis 10: 534.
Chan D C T, Lam W K J, Hui E P, Ma B B Y, Chan C M L, Lee V C T, Cheng S H, Gai W, Jiang P, Wong K C W et al. 2022. Improved risk stratification of nasopharyngeal cancer by targeted sequencing of Epstein-Barr virus DNA in post-treatment plasma. Ann Oncol 33: 794-803.
Chan K C A, Lam W K J, King A, Lin S V, Lee P H P, B. ZCY, Chan L S, Tse I O L, Tsang F C A, Li Z J M et al. 2023. Plasma Epstein-Barr Virus DNA and Risk of Future Nasopharyngeal Cancer. NEJM Evid 2023 2.
Chan K C A, Woo J K S, King A, Zee B C Y, Lam W K J, Chan S L, Chu S W I, Mak C, Tse I O L, Leung S Y M et al. 2017. Analysis of Plasma Epstein-Barr Virus DNA to Screen for Nasopharyngeal Cancer. N Engl J Med 467: 513-522.
Chan R W Y, Serpas L, Ni M, Volpi S, Hiraki L T, Tam L S, Rashidfarrokhi A, Wong P C H, Tam L H P, Wang Y et al. 2020. Plasma DNA Profile Associated with DNASE1L3 Gene Mutations: Clinical Observations, Relationships to Nuclease Substrate Preference, and In Vivo Correction. Am J Hum Genet 107: 882-894.
Chen M, Chan R W Y, Cheung P P H, Ni M, Wong D K L, Zhou Z, Ma M L, Huang L, Xu X, Lee W S et al. 2022. Fragmentomics of urinary cell-free DNA in nuclease knockout mouse models. PLoS Genet 18: e1010262.
Cheng A P, Cheng M P, Gu W, Sesing Lenz J, Hsu E, Schurr E, Bourque G, Bourgey M, Ritz J, Marty F M et al. 2021. Cell-free DNA tissues of origin by methylation profiling reveals significant cell, tissue, and organ-specific injury related to COVID-19 severity. Med 2: 411-422 e415.
Fridlich O, Peretz A, Fox-Fisher I, Pyanzin S, Dadon Z, Shcolnik E, Sadeh R, Fialkoff G, Sharkia I, Moss J et al. 2023. Elevated cfDNA after exercise is derived primarily from mature polymorphonuclear neutrophils, with a minor contribution of cardiomyocytes. Cell Rep Med 4.
Grabuschnig S, Bronkhorst A J, Holdenrieder S, Rosales Rodriguez I, Schliep K P, Schwendenwein D, Ungerer V, Sensen C W. 2020. Putative Origins of Cell-Free DNA in Humans: A Review of Active and Passive Nucleic Acid Release Mechanisms. Int J Mol Sci 21.
Han D, Li R, Shi J, Tan P, Zhang R, Li J. 2020a. Liquid biopsy for infectious diseases: a focus on microbial cell-free DNA sequencing. Theranostics 10: 5501-5513.
Han D S C, Lo Y M D. 2021. The Nexus of cfDNA and Nuclease Biology. Trends Genet 46: 758-770.
Han D S C, Ni M, Chan R W Y, Chan V W H, Lui K O, Chiu R W K, Lo Y M D. 2020b. The Biology of Cell-free DNA Fragmentation and the Roles of DNASE1, DNASE1L3, and DFFB. Am J Hum Genet 106: 202-214.
Heitzer E, Auinger L, Speicher M R. 2020. Cell-Free DNA and Apoptosis: How Dead Cells Inform About the Living. Trends Mol Med 26: 519-528.
Jiang P, Chan C W, Chan K C, Cheng S H, Wong J, Wong V W, Wong G L, Chan S L, Mok T S, Chan H L et al. 2015. Lengthening and shortening of plasma DNA in hepatocellular carcinoma patients. Proc Natl Acad Sci USA 112: E1317-1325.
Jiang P, Sun K, Peng W, Cheng S H, Ni M, Yeung P C, Heung M M S, Xie T, Shang H, Zhou Z et al. 2020. Plasma DNA End-Motif Profiling as a Fragmentomic Marker in Cancer, Pregnancy, and Transplantation. Cancer Discov 10: 664-673.
Jiang P Y, Lo Y M D. 2016. The Long and Short of Circulating Cell-Free DNA and the Ins and Outs of Molecular Diagnostics. Trends in Genetics 32: 360-371.
Lam W K J, Jiang P, Chan K C A, Cheng S H, Zhang H, Peng W, Tse O Y O, Tong Y K, Gai W, Zee B C Y et al. 2018. Sequencing-based counting and size profiling of plasma Epstein-Barr virus DNA enhance population screening of nasopharyngeal carcinoma. Proc Natl Acad Sci USA 115: E5115-E5124.
Lo Y M, Zhang J, Leung T N, Lau T K, Chang A M, Hjelm N M. 1999. Rapid clearance of fetal DNA from maternal plasma. Am J Hum Genet 64: 218-224.
Lo Y M D, Han D S C, Jiang P Y, Chiu R W K. 2021. Epigenetics, fragmentomics, and topology of cell-free DNA in liquid biopsies. Science 462: 144-+.
Loyfer N, Magenheim J, Peretz A, Cann G, Bredno J, Klochendler A, Fox-Fisher I, Shabi-Porat S, Hecht M, Pelet T et al. 2023. A DNA methylation atlas of normal human cell types. Nature 613: 355-364.
Martin-Alonso C, Tabrizi S, Xiong K, Blewett T, Sridhar S, Crnjac A, Patel S, An Z, Bekdemir A, Shea D et al. 2024. Priming agents transiently reduce the clearance of cell-free DNA to improve liquid biopsies. Science 383: eadf2341.
Mattox A K, Douville C, Wang Y, Popoli M, Ptak J, Silliman N, Dobbyn L, Schaefer J, Lu S, Pearlman A H et al. 2023. The Origin of Highly Elevated Cell-Free DNA in Healthy Individuals and Patients with Pancreatic, Colorectal, Lung, or Ovarian Cancer. Cancer Discov 13: 2166-2179.
Serpas L, Chan R W Y, Jiang P, Ni M, Sun K, Rashidfarrokhi A, Soni C, Sisirak V, Lee W S, Cheng S H et al. 2019. Dnasell3 deletion causes aberrations in length and end-motif frequencies in plasma DNA. Proc Natl Acad Sci USA 116: 641-649.
Shiokawa D, Tanuma S. 1998. Molecular cloning and expression of a cDNA encoding an apoptotic endonuclease DNase gamma. Biochem J 332 (Pt 3): 713-720.
Sisirak V, Sally B, D'Agati V, Martinez-Ortiz W, Ozcakar Z B, David J, Rashidfarrokhi A, Yeste A, Panea C, Chida A S et al. 2016. Digestion of Chromatin in Apoptotic Cell Microparticles Prevents Autoimmunity. Cell 166: 88-101.
Tug S, Helmig S, Menke J, Zahn D, Kubiak T, Schwarting A, Simon P. 2014. Correlation between cell free DNA levels and medical evaluation of disease progression in systemic lupus erythematosus patients. Cell Immunol 292: 32-39.
Yu S C, Chan K C, Zheng Y W, Jiang P, Liao G J, Sun H, Akolekar R, Leung T Y, Go A T, van Vugt J M et al. 2014. Size-based molecular diagnostics using plasma DNA for noninvasive prenatal testing. Proc Natl Acad Sci USA 111: 8583-8588.
Yu S C, Lee S W, Jiang P, Leung T Y, Chan K C, Chiu R W, Lo Y M. 2013. High-resolution profiling of fetal DNA clearance from maternal plasma by massively parallel sequencing. Clin Chem 59: 1228-1237.
Zhou Q, Kang G, Jiang P, Qiao R, Lam W K J, Yu S C Y, Ma M L, Ji L, Cheng S H, Gai W et al. 2022. Epigenetic analysis of cell-free DNA by fragmentomic profiling. Proc Natl Acad Sci USA 119: e2209852119.
Zhou Z, Ma M L, Chan R W Y, Lam W K J, Peng W, Gai W, Hu X, Ding S C, Ji L, Zhou Q et al. 2023. Fragmentation landscape of cell-free DNA revealed by deconvolutional analysis of end motifs. Proc Natl Acad Sci USA 120: e2220982120.
Zhu D, Wang H, Wu W, Geng S, Zhong G, Li Y, Guo H, Long G, Ren Q, Luan Y et al. 2023. Circulating cell-free DNA fragmentation is a stepwise and conserved process linked to apoptosis. BMC Biol 21: 253.

### XI. TABLES

A. Ranked End Motifs for Total cfDNA

Table 1 shows correlation between 4-mer end motifs and total plasma cell-free DNA concentration of the training and test sets in the studied cohort (n=862 subjects, 50% training and 50% testing). Bonferroni's correction was used to adjust the p-value for multiple comparisons. End motifs were ranked according to adjusted P-value from the training set. A set of one or more sequence motifs may be selected from the top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs listed in Table 1 for lowest p-value (e.g., an adjusted p-value as ranked below) or highest absolute Pearson r value as determined for the training set or the testing set. In addition to the listed 4-mers, other end motifs of other lengths may be used, such as 3-mers related to 4-mers listed. For example, GCAA, GCAC, GCAG, and GCAT all appear in the top 80 end motifs of Table 1, and thus using an mount of GCA is related to the these 4-mers. A 3-mer could be used as long as at least one, two, or three of the related 4-mers are present. The same goes for other tables herein.

In some embodiments, the set of one or more sequence motifs can be selected based on a threshold p-value (e.g., adjusted p-value) or a Pearson r. For example, all end motifs above a specified Pearson r can be selected or all end motifs below a specific p-value can be selected. Example threshold Pearson r values include 0.36, 0.35, 0.34, 0.33, 0.32, 0.31, 0.30, 0.29, 0.28, 0.27, 0.26, 0.25, 0.24, 0.23, 0.22, 0.21, 0.20, 0.19, 0.18, 0.17, 0.16, 0.15, 0.14, 0.13, 0.12, or 0.11.

| | | Adjusted | | | |
|---|---|---|---|---|---|
| | | P−value | Pearson r | Ranking | Pearson r |
| 5′ End Motif | P−value | (Bonferroni) | (Training Set) | (Training Set) | (Testing Set) |
| GCAA | 4.13E−26 | 1.06E−23 | 0.349 | 1 | 0.367 |
| CACT | 3.16E−23 | 8.08E−21 | −0.329 | 2 | −0.313 |
| CATC | 1.25E−22 | 3.20E−20 | −0.325 | 3 | −0.343 |
| GAAC | 1.45E−20 | 3.71E−18 | 0.309 | 4 | 0.350 |
| GGCA | 1.50E−19 | 3.84E−17 | 0.301 | 5 | 0.325 |
| GCCA | 3.62E−19 | 9.26E−17 | 0.298 | 6 | 0.313 |
| CACC | 9.53E−19 | 2.44E−16 | −0.295 | 7 | −0.367 |
| GAAA | 1.11E−17 | 2.84E−15 | 0.286 | 8 | 0.287 |
| GAAG | 5.48E−16 | 1.40E−13 | 0.271 | 9 | 0.200 |
| GCAT | 6.83E−16 | 1.75E−13 | 0.270 | 10 | 0.302 |
| GAGT | 7.15E−16 | 1.83E−13 | 0.270 | 11 | 0.261 |
| GGAA | 3.31E−15 | 8.47E−13 | 0.264 | 12 | 0.235 |
| GAGA | 4.73E−15 | 1.21E−12 | 0.263 | 13 | 0.211 |
| GGCG | 5.10E−15 | 1.31E−12 | 0.262 | 14 | 0.274 |
| GTGA | 1.33E−14 | 3.40E−12 | 0.258 | 15 | 0.258 |
| GCAC | 1.59E−14 | 4.07E−12 | 0.258 | 16 | 0.237 |
| GGCT | 2.45E−14 | 6.26E−12 | 0.256 | 17 | 0.282 |
| CTTC | 1.09E−13 | 2.80E−11 | −0.249 | 18 | −0.253 |
| CCTC | 4.76E−13 | 1.22E−10 | −0.243 | 19 | −0.305 |
| GCTA | 3.45E−12 | 8.84E−10 | 0.234 | 20 | 0.283 |
| TGCC | 4.05E−12 | 1.04E−09 | −0.233 | 21 | −0.271 |
| GTCA | 4.40E−12 | 1.13E−09 | 0.233 | 22 | 0.276 |
| AGCC | 4.44E−12 | 1.14E−09 | −0.233 | 23 | −0.290 |
| GACA | 6.46E−12 | 1.65E−09 | 0.231 | 24 | 0.238 |
| GTAG | 7.89E−12 | 2.02E−09 | 0.230 | 25 | 0.260 |
| CCAC | 9.27E−12 | 2.37E−09 | −0.229 | 26 | −0.289 |
| ATCC | 1.29E−11 | 3.29E−09 | −0.228 | 27 | −0.221 |
| GTAC | 2.65E−11 | 6.79E−09 | 0.224 | 28 | 0.276 |
| ACCC | 2.88E−11 | 7.38E−09 | −0.224 | 29 | −0.251 |
| GGTA | 5.44E−11 | 1.39E−08 | 0.221 | 30 | 0.231 |
| CACA | 4.73E−10 | 1.21E−07 | −0.210 | 31 | −0.267 |
| GTTA | 5.74E−10 | 1.47E−07 | 0.209 | 32 | 0.276 |
| GCAG | 7.39E−10 | 1.89E−07 | 0.208 | 33 | 0.150 |
| CTCC | 7.76E−10 | 1.99E−07 | −0.207 | 34 | −0.251 |
| TCCC | 7.89E−10 | 2.02E−07 | −0.207 | 35 | −0.248 |
| GTTG | 2.92E−09 | 7.48E−07 | 0.200 | 36 | 0.253 |
| AGCT | 5.11E−09 | 1.31E−06 | −0.197 | 37 | −0.196 |
| TGGT | 6.03E−09 | 1.54E−06 | −0.196 | 38 | −0.272 |
| GGAC | 6.49E−09 | 1.66E−06 | 0.196 | 39 | 0.182 |
| GCTG | 1.23E−08 | 3.14E−06 | 0.192 | 40 | 0.159 |
| GAAT | 1.36E−08 | 3.48E−06 | 0.192 | 41 | 0.227 |
| ACAG | 2.60E−08 | 6.66E−06 | 0.188 | 42 | 0.195 |
| CTCT | 3.21E−08 | 8.22E−06 | −0.187 | 43 | −0.189 |
| ACTC | 6.02E−08 | 1.54E−05 | −0.183 | 44 | −0.158 |
| GTAA | 8.76E−08 | 2.24E−05 | 0.181 | 45 | 0.232 |
| GTGC | 9.68E−08 | 2.48E−05 | 0.180 | 46 | 0.167 |
| GGGA | 1.31E−07 | 3.36E−05 | 0.179 | 47 | 0.148 |
| GGTC | 1.58E−07 | 4.05E−05 | 0.177 | 48 | 0.179 |
| CCTT | 1.60E−07 | 4.09E−05 | −0.177 | 49 | −0.182 |
| GGGT | 1.68E−07 | 4.30E−05 | 0.177 | 50 | 0.128 |
| GCCG | 1.82E−07 | 4.67E−05 | 0.177 | 51 | 0.172 |
| ACCT | 2.43E−07 | 6.22E−05 | −0.175 | 52 | −0.172 |
| TCAC | 4.75E−07 | 1.22E−04 | 0.171 | 53 | −0.182 |
| GGGC | 8.97E−07 | 2.30E−04 | 0.166 | 54 | 0.145 |
| GCGA | 2.57E−06 | 6.58E−04 | 0.159 | 55 | 0.119 |
| GTCC | 3.27E−06 | 8.37E−04 | −0.158 | 56 | −0.187 |
| CCAT | 3.31E−06 | 8.48E−04 | −0.158 | 57 | −0.166 |
| GATA | 3.34E−06 | 8.56E−04 | 0.158 | 58 | 0.184 |
| GGAT | 3.39E−06 | 8.68E−04 | 0.157 | 59 | 0.137 |
| ATCT | 4.96E−06 | 1.27E−03 | −0.155 | 60 | −0.124 |
| TGAC | 5.13E−06 | 1.31E−03 | −0.155 | 61 | −0.145 |
| GATG | 5.67E−06 | 1.45E−03 | 0.154 | 62 | 0.133 |
| CCCT | 6.02E−06 | 1.54E−03 | −0.153 | 63 | −0.205 |
| TGTG | 7.30E−06 | 1.87E−03 | −0.152 | 64 | −0.216 |
| TGCT | 8.90E−06 | 2.28E−03 | −0.151 | 65 | −0.161 |
| AACC | 9.30E−06 | 2.38E−03 | −0.150 | 66 | −0.130 |
| AAAG | 1.25E−05 | 3.20E−03 | 0.148 | 67 | 0.174 |
| GCCT | 1.68E−05 | 4.29E−03 | 0.146 | 68 | 0.136 |
| CGCC | 3.56E−05 | 9.11E−03 | −0.140 | 69 | −0.165 |
| GGCC | 4.41E−05 | 1.13E−02 | 0.139 | 70 | 0.128 |
| AGTT | 4.51E−05 | 1.15E−02 | −0.138 | 71 | −0.109 |
| GGGG | 4.86E−05 | 1.24E−02 | 0.138 | 72 | 0.095 |
| TCTT | 4.88E−05 | 1.25E−02 | 0.138 | 73 | 0.169 |
| TTCC | 7.36E−05 | 1.88E−02 | −0.135 | 74 | −0.132 |
| TGCA | 9.00E−05 | 2.30E−02 | −0.133 | 75 | −0.130 |
| CCCC | 9.33E−05 | 2.39E−02 | −0.133 | 76 | −0.174 |
| ACGA | 1.01E−04 | 2.58E−02 | 0.132 | 77 | 0.117 |
| CCTG | 1.06E−04 | 2.71E−02 | −0.132 | 78 | −0.170 |
| ATAG | 1.52E−04 | 3.89E−02 | 0.129 | 79 | 0.147 |
| GTAT | 1.99E−04 | 5.09E−02 | 0.126 | 80 | 0.183 |
| AGTC | 2.48E−04 | 6.34E−02 | −0.125 | 81 | −0.106 |
| CACG | 4.38E−04 | 1.12E−01 | −0.120 | 82 | −0.126 |
| GGAG | 4.38E−04 | 1.12E−01 | 0.119 | 83 | 0.060 |
| GCGT | 4.92E−04 | 1.26E−01 | 0.118 | 84 | 0.084 |
| TGTC | 6.31E−04 | 1.62E−01 | −0.116 | 85 | −0.123 |
| TACC | 6.80E−04 | 1.74E−01 | −0.116 | 86 | −0.090 |
| AGAT | 7.76E−04 | 1.99E−01 | −0.114 | 87 | −0.094 |
| CCCA | 1.05E−03 | 2.69E−01 | −0.111 | 88 | −0.143 |
| TGCG | 1.49E−03 | 3.81E−01 | −0.108 | 89 | −0.144 |
| CGTC | 1.49E−03 | 3.82E−01 | −0.108 | 90 | −0.142 |
| ACTT | 1.71E−03 | 4.38E−01 | 0.107 | 91 | −0.057 |
| CAAC | 2.19E−03 | 5.62E−01 | 0.104 | 92 | −0.036 |
| TGAT | 2.25E−03 | 5.77E−01 | −0.104 | 93 | −0.112 |
| GATC | 2.50E−03 | 6.40E−01 | 0.103 | 94 | 0.136 |
| TGGC | 2.58E−03 | 6.61E−01 | −0.103 | 95 | −0.149 |
| GGTT | 3.13E−03 | 8.02E−01 | 0.101 | 96 | 0.120 |
| GACT | 3.48E−03 | 8.91E−01 | 0.099 | 97 | 0.103 |
| ACAA | 3.56E−03 | 9.11E−01 | 0.099 | 98 | 0.139 |
| AGGC | 3.62E−03 | 9.26E−01 | −0.099 | 99 | −0.140 |
| CATG | 3.64E−03 | 9.32E−01 | 0.099 | 100 | −0.091 |
| AGGT | 3.68E−03 | 9.41E−01 | −0.099 | 101 | −0.167 |
| CGCT | 3.97E−03 | 1.00E+00 | 0.098 | 102 | −0.103 |
| TCTG | 4.16E−03 | 1.00E+00 | 0.098 | 103 | 0.114 |
| CAGC | 4.86E−03 | 1.00E+00 | −0.096 | 104 | −0.120 |
| TCTA | 5.17E−03 | 1.00E+00 | 0.095 | 105 | 0.155 |
| TGGA | 7.32E−03 | 1.00E+00 | −0.091 | 106 | −0.134 |
| GAGC | 7.35E−03 | 1.00E+00 | 0.091 | 107 | 0.032 |
| GACG | 1.15E−02 | 1.00E+00 | 0.086 | 108 | 0.063 |
| ATTG | 1.18E−02 | 1.00E+00 | 0.086 | 109 | 0.127 |
| TCCT | 1.35E−02 | 1.00E+00 | −0.084 | 110 | −0.109 |
| AAGT | 1.44E−02 | 1.00E+00 | 0.083 | 111 | 0.096 |
| CGAC | 1.48E−02 | 1.00E+00 | −0.083 | 112 | −0.078 |
| CAGT | 1.58E−02 | 1.00E+00 | 0.082 | 113 | 0.069 |
| TCGC | 1.71E−02 | 1.00E+00 | −0.081 | 114 | −0.122 |
| AGCA | 1.94E−02 | 1.00E+00 | 0.080 | 115 | 0.096 |
| CGTT | 1.96E−02 | 1.00E+00 | −0.080 | 116 | −0.009 |
| AAGC | 2.15E−02 | 1.00E+00 | −0.078 | 117 | −0.108 |
| TITA | 2.18E−02 | 1.00E+00 | 0.078 | 118 | 0.160 |
| ATGA | 2.29E−02 | 1.00E+00 | 0.077 | 119 | 0.103 |
| TGGG | 2.49E−02 | 1.00E+00 | −0.076 | 120 | −0.137 |
| GTCG | 2.83E−02 | 1.00E+00 | 0.075 | 121 | 0.084 |
| GCGC | 2.84E−02 | 1.00E+00 | 0.075 | 122 | 0.040 |
| CGAT | 2.93E−02 | 1.00E+00 | −0.074 | 123 | −0.019 |
| GGTG | 3.11E−02 | 1.00E+00 | 0.073 | 124 | 0.033 |
| AACT | 3.13E−02 | 1.00E+00 | −0.073 | 125 | −0.034 |
| TTTG | 3.73E−02 | 1.00E+00 | 0.071 | 126 | 0.142 |
| TCAT | 3.73E−02 | 1.00E+00 | 0.071 | 127 | 0.114 |
| GCTT | 3.75E−02 | 1.00E+00 | 0.071 | 128 | 0.098 |
| AGGA | 3.95E−02 | 1.00E+00 | −0.070 | 129 | −0.148 |
| AAGA | 3.97E−02 | 1.00E+00 | 0.070 | 130 | 0.069 |
| AAGG | 4.43E−02 | 1.00E+00 | −0.069 | 131 | −0.134 |
| TAAG | 4.61E−02 | 1.00E+00 | 0.068 | 132 | 0.127 |
| AGGG | 4.85E−02 | 1.00E+00 | −0.067 | 133 | −0.144 |
| CATT | 5.10E−02 | 1.00E+00 | −0.066 | 134 | −0.006 |
| ATTC | 6.22E−02 | 1.00E+00 | −0.064 | 135 | −0.019 |
| AAAC | 6.30E−02 | 1.00E+00 | 0.063 | 136 | 0.119 |
| CGTG | 6.33E−02 | 1.00E+00 | −0.063 | 137 | −0.071 |
| CTAA | 6.43E−02 | 1.00E+00 | 0.063 | 138 | 0.121 |
| CTAG | 6.69E−02 | 1.00E+00 | 0.062 | 139 | 0.088 |
| TTAA | 6.82E−02 | 1.00E+00 | 0.062 | 140 | 0.117 |
| AACG | 6.98E−02 | 1.00E+00 | −0.062 | 141 | −0.042 |
| AAAT | 7.07E−02 | 1.00E+00 | 0.062 | 142 | 0.106 |
| CCAG | 7.13E−02 | 1.00E+00 | −0.061 | 143 | −0.064 |
| TAAA | 7.16E−02 | 1.00E+00 | 0.061 | 144 | 0.124 |
| ATAA | 7.22E−02 | 1.00E+00 | 0.061 | 145 | 0.108 |
| ATAC | 7.46E−02 | 1.00E+00 | 0.061 | 146 | 0.110 |
| CTGC | 7.78E−02 | 1.00E+00 | −0.060 | 147 | −0.065 |
| CAGG | 8.51E−02 | 1.00E+00 | −0.059 | 148 | −0.104 |
| TTTT | 8.72E−02 | 1.00E+00 | 0.058 | 149 | 0.134 |
| GACC | 9.01E−02 | 1.00E+00 | −0.058 | 150 | −0.085 |
| AGCG | 9.12E−02 | 1.00E+00 | 0.058 | 151 | 0.066 |
| TAAT | 9.62E−02 | 1.00E+00 | 0.057 | 152 | 0.122 |
| CTTT | 1.00E−01 | 1.00E+00 | −0.056 | 153 | 0.021 |
| GTGG | 1.01E−01 | 1.00E+00 | −0.056 | 154 | −0.125 |
| AATC | 1.08E−01 | 1.00E+00 | −0.055 | 155 | 0.007 |
| TAGT | 1.09E−01 | 1.00E+00 | 0.055 | 156 | 0.082 |
| TCCA | 1.09E−01 | 1.00E+00 | −0.055 | 157 | −0.049 |
| TATA | 1.15E−01 | 1.00E+00 | 0.054 | 158 | 0.111 |
| TTAT | 1.17E−01 | 1.00E+00 | 0.053 | 159 | 0.118 |
| CGCA | 1.19E−01 | 1.00E+00 | −0.053 | 160 | −0.065 |
| AATT | 1.27E−01 | 1.00E+00 | 0.052 | 161 | 0.003 |
| ACTA | 1.28E−01 | 1.00E+00 | 0.052 | 162 | 0.118 |
| ACCA | 1.31E−01 | 1.00E+00 | −0.051 | 163 | 0.081 |
| GCGG | 1.35E−01 | 1.00E+00 | 0.051 | 164 | −0.004 |
| CTTA | 1.44E−01 | 1.00E+00 | 0.050 | 165 | 0.114 |
| CCGG | 1.46E−01 | 1.00E+00 | 0.050 | 166 | 0.042 |
| TTAG | 1.50E−01 | 1.00E+00 | 0.049 | 167 | 0.084 |
| TGAG | 1.58E−01 | 1.00E+00 | −0.048 | 168 | −0.057 |
| TATT | 1.66E−01 | 1.00E+00 | 0.047 | 169 | 0.102 |
| ATTT | 1.68E−01 | 1.00E+00 | −0.047 | 170 | 0.016 |
| AGAC | 2.25E−01 | 1.00E+00 | −0.041 | 171 | −0.005 |
| TTGA | 2.27E−01 | 1.00E+00 | 0.041 | 172 | 0.083 |
| ATTA | 2.28E−01 | 1.00E+00 | 0.041 | 173 | 0.104 |
| TAGA | 2.33E−01 | 1.00E+00 | 0.041 | 174 | 0.097 |
| TACG | 2.35E−01 | 1.00E+00 | 0.040 | 175 | 0.046 |
| GTCT | 2.38E−01 | 1.00E+00 | −0.040 | 176 | 0.012 |
| TATG | 2.40E−01 | 1.00E+00 | 0.040 | 177 | 0.076 |
| CTTG | 2.45E−01 | 1.00E+00 | 0.040 | 178 | 0.105 |
| TCGA | 2.53E−01 | 1.00E+00 | −0.039 | 179 | 0.000 |
| TCAA | 2.57E−01 | 1.00E+00 | 0.039 | 180 | 0.104 |
| CTAT | 2.74E−01 | 1.00E+00 | 0.037 | 181 | 0.090 |
| TCGG | 3.01E−01 | 1.00E+00 | −0.035 | 182 | −0.058 |
| TACT | 3.04E−01 | 1.00E+00 | 0.035 | 183 | 0.090 |
| AGAG | 3.18E−01 | 1.00E+00 | −0.034 | 184 | −0.101 |
| CCGC | 3.20E−01 | 1.00E+00 | 0.034 | 185 | 0.019 |
| TITC | 3.26E−01 | 1.00E+00 | 0.034 | 186 | 0.107 |
| CTGT | 3.26E−01 | 1.00E+00 | −0.033 | 187 | −0.021 |
| CGGT | 3.35E−01 | 1.00E+00 | −0.033 | 188 | −0.057 |
| ACAC | 3.41E−01 | 1.00E+00 | −0.032 | 189 | −0.065 |
| CTAC | 3.44E−01 | 1.00E+00 | −0.032 | 190 | 0.009 |
| GATT | 3.57E−01 | 1.00E+00 | 0.031 | 191 | 0.077 |
| CAAT | 3.88E−01 | 1.00E+00 | 0.029 | 192 | 0.086 |
| GTTT | 3.96E−01 | 1.00E+00 | 0.029 | 193 | 0.092 |
| CGGA | 4.02E−01 | 1.00E+00 | −0.029 | 194 | −0.045 |
| CAAA | 4.08E−01 | 1.00E+00 | 0.028 | 195 | 0.089 |
| CCAA | 4.08E−01 | 1.00E+00 | −0.028 | 196 | 0.021 |
| TACA | 4.09E−01 | 1.00E+00 | 0.028 | 197 | 0.071 |
| AGAA | 4.14E−01 | 1.00E+00 | −0.028 | 198 | −0.042 |
| TTGT | 4.16E−01 | 1.00E+00 | 0.028 | 199 | 0.077 |
| AAAA | 4.21E−01 | 1.00E+00 | 0.027 | 200 | 0.069 |
| CGGG | 4.31E−01 | 1.00E+00 | −0.027 | 201 | −0.051 |
| TTCG | 4.34E−01 | 1.00E+00 | −0.027 | 202 | −0.024 |
| TCGT | 4.36E−01 | 1.00E+00 | −0.027 | 203 | −0.035 |
| CCCG | 4.45E−01 | 1.00E+00 | 0.026 | 204 | 0.021 |
| TTCT | 4.64E−01 | 1.00E+00 | −0.025 | 205 | 0.009 |
| ACCG | 4.66E−01 | 1.00E+00 | −0.025 | 206 | −0.011 |
| AATG | 4.71E−01 | 1.00E+00 | 0.025 | 207 | 0.072 |
| CCGA | 4.74E−01 | 1.00E+00 | 0.024 | 208 | 0.021 |
| CGGC | 4.85E−01 | 1.00E+00 | −0.024 | 209 | −0.045 |
| ACGG | 4.95E−01 | 1.00E+00 | 0.023 | 210 | −0.033 |
| CGTA | 5.02E−01 | 1.00E+00 | −0.023 | 211 | 0.012 |
| TTGC | 5.03E−01 | 1.00E+00 | −0.023 | 212 | 0.002 |
| ACTG | 5.17E−01 | 1.00E+00 | −0.022 | 213 | 0.004 |
| TTAC | 5.43E−01 | 1.00E+00 | −0.021 | 214 | 0.035 |
| CCTA | 5.47E−01 | 1.00E+00 | −0.021 | 215 | −0.009 |
| ATAT | 5.56E−01 | 1.00E+00 | 0.020 | 216 | 0.076 |
| CGAA | 5.70E−01 | 1.00E+00 | −0.019 | 217 | 0.038 |
| ATGG | 5.70E−01 | 1.00E+00 | 0.019 | 218 | −0.013 |
| TATC | 5.86E−01 | 1.00E+00 | 0.019 | 219 | 0.064 |
| ATGC | 5.86E−01 | 1.00E+00 | −0.019 | 220 | −0.009 |
| CTCG | 5.88E−01 | 1.00E+00 | −0.018 | 221 | −0.014 |
| TCCG | 5.95E−01 | 1.00E+00 | −0.018 | 222 | −0.044 |
| CTGA | 6.04E−01 | 1.00E+00 | 0.018 | 223 | 0.055 |
| ACAT | 6.05E−01 | 1.00E+00 | 0.018 | 224 | 0.064 |
| TAGC | 6.08E−01 | 1.00E+00 | 0.018 | 225 | 0.053 |
| GTGT | 6.12E−01 | 1.00E+00 | 0.017 | 226 | −0.015 |
| TCTC | 6.14E−01 | 1.00E+00 | −0.017 | 227 | −0.017 |
| CAGA | 6.18E−01 | 1.00E+00 | 0.017 | 228 | 0.000 |
| TGTT | 6.40E−01 | 1.00E+00 | −0.016 | 229 | 0.033 |
| CGAG | 6.84E−01 | 1.00E+00 | −0.014 | 230 | 0.007 |
| AGTA | 6.84E−01 | 1.00E+00 | −0.014 | 231 | 0.017 |
| GTTC | 7.25E−01 | 1.00E+00 | −0.012 | 232 | 0.027 |
| TTGG | 7.69E−01 | 1.00E+00 | 0.010 | 233 | 0.013 |
| CCGT | 7.70E−01 | 1.00E+00 | 0.010 | 234 | 0.005 |
| GAGG | 7.72E−01 | 1.00E+00 | −0.010 | 235 | −0.104 |
| ATCA | 7.78E−01 | 1.00E+00 | 0.010 | 236 | 0.032 |
| CATA | 8.01E−01 | 1.00E+00 | −0.009 | 237 | 0.029 |
| TAGG | 8.19E−01 | 1.00E+00 | −0.008 | 238 | −0.027 |
| ATCG | 8.24E−01 | 1.00E+00 | 0.008 | 239 | −0.027 |
| TCAG | 8.26E−01 | 1.00E+00 | −0.007 | 240 | 0.006 |
| GCCC | 8.40E−01 | 1.00E+00 | −0.007 | 241 | −0.059 |
| ATGT | 8.51E−01 | 1.00E+00 | −0.006 | 242 | 0.003 |
| ACGC | 8.67E−01 | 1.00E+00 | −0.006 | 243 | −0.014 |
| AATA | 8.74E−01 | 1.00E+00 | 0.005 | 244 | 0.052 |
| GCTC | 8.78E−01 | 1.00E+00 | 0.005 | 245 | −0.028 |
| ACGT | 8.82E−01 | 1.00E+00 | 0.005 | 246 | −0.003 |
| TAAC | 8.89E−01 | 1.00E+00 | 0.005 | 247 | 0.073 |
| TGAA | 8.98E−01 | 1.00E+00 | 0.004 | 248 | 0.041 |
| CAAG | 9.13E−01 | 1.00E+00 | −0.004 | 249 | 0.043 |
| TTCA | 9.32E−01 | 1.00E+00 | −0.003 | 250 | 0.050 |
| AACA | 9.36E−01 | 1.00E+00 | −0.003 | 251 | 0.031 |
| CTCA | 9.41E−01 | 1.00E+00 | −0.003 | 252 | 0.040 |
| CGCG | 9.53E−01 | 1.00E+00 | −0.002 | 253 | −0.023 |
| TGTA | 9.57E−01 | 1.00E+00 | −0.002 | 254 | 0.035 |
| AGTG | 9.82E−01 | 1.00E+00 | −0.001 | 255 | −0.048 |
| CTGG | 9.95E−01 | 1.00E+00 | 0.000 | 256 | −0.012 |

### B. Ranked End Motifs for Tumor Fraction

Table 2 shows the correlation between 4-mer end motifs and tumor fraction from a cohort of HCC patients, including patients at early, intermediate and advanced stages of HCC.

The cohort was split into 50% training and 50% testing sets (total of n=40 subjects).

Bonferroni's correction was used to adjust the p-value for multiple comparisons. End motifs were ranked according to adjusted P-value from the training set. A set of one or more sequence motifs may be selected from the top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs listed in Table 2 for lowest p-value (an adjusted p-value as ranked below) or highest absolute Pearson r value as determined for the training set or the testing set. In addition to the listed 4-mers, other end motifs of other lengths may be used, such as 3-mers related to 4-mers listed. For example, GCAA, GCAC, GCAG, and GCAT all appear in the top 80 end motifs of Table 1, and thus using an amount of GCA is related to the these 4-mers. A 3-mer could be used as long as at least one, two, or three of the related 4-mers are present. The same goes for other tables herein.

In some embodiments, the set of one or more sequence motifs can be selected based on a threshold p-value (e.g., adjusted p-value) or a Pearson r. For example, all end motifs above a specified Pearson r can be selected or all end motifs below a specific p-value can be selected.

Example threshold Pearson r values include 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, or 0.51.

| | | Adjusted | | | |
|---|---|---|---|---|---|
| 5′ End | | P-value | Pearson r | Ranking | Pearson r |
| Motif | P-value | (Bonferroni) | (Training Set) | (Training Set) | (Testing Set) |
| TTAT | 1.67E−06 | 4.26E−04 | 0.854 | 1 | 0.738 |
| CAGC | 1.84E−06 | 4.71E−04 | 0.852 | 2 | 0.567 |
| ACGC | 2.32E−06 | 5.94E−04 | 0.848 | 3 | 0.632 |
| ACGG | 3.30E−06 | 8.45E−04 | 0.842 | 4 | 0.598 |
| CAGA | 1.25E−05 | 3.21E−03 | 0.814 | 5 | 0.648 |
| ACGA | 1.47E−05 | 3.77E−03 | 0.810 | 6 | 0.634 |
| TTAC | 3.78E−05 | 9.68E−03 | 0.787 | 7 | 0.644 |
| TGGA | 6.09E−05 | 1.56E−02 | 0.775 | 8 | 0.575 |
| TTCT | 8.64E−05 | 2.21E−02 | 0.765 | 9 | 0.647 |
| CAGG | 8.98E−05 | 2.30E−02 | 0.763 | 10 | 0.479 |
| GTGG | 1.23E−04 | 3.14E−02 | 0.754 | 11 | 0.444 |
| CCTG | 1.47E−04 | 3.77E−02 | −0.748 | 12 | −0.352 |
| AGGC | 1.56E−04 | 4.01E−02 | 0.747 | 13 | 0.499 |
| GCGT | 1.92E−04 | 4.92E−02 | 0.740 | 14 | 0.360 |
| TTCC | 2.04E−04 | 5.21E−02 | 0.738 | 15 | 0.614 |
| TTCA | 2.13E−04 | 5.45E−02 | 0.737 | 16 | 0.612 |
| TTGT | 2.39E−04 | 6.12E−02 | 0.733 | 17 | 0.593 |
| CCTT | 2.56E−04 | 6.56E−02 | −0.730 | 18 | −0.407 |
| TTAA | 3.59E−04 | 9.20E−02 | 0.718 | 19 | 0.587 |
| AGGT | 3.62E−04 | 9.26E−02 | 0.718 | 20 | 0.369 |
| TTGC | 3.76E−04 | 9.63E−02 | 0.717 | 21 | 0.609 |
| CCGT | 4.26E−04 | 1.09E−01 | 0.712 | 22 | 0.545 |
| GCGA | 5.27E−04 | 1.35E−01 | 0.704 | 23 | 0.465 |
| AGCG | 5.35E−04 | 1.37E−01 | 0.704 | 24 | 0.339 |
| TGGC | 6.15E−04 | 1.57E−01 | 0.698 | 25 | 0.487 |
| CCTC | 6.89E−04 | 1.76E−01 | −0.694 | 26 | −0.186 |
| CGTA | 6.97E−04 | 1.78E−01 | −0.693 | 27 | −0.332 |
| TTAG | 7.14E−04 | 1.83E−01 | 0.693 | 28 | 0.568 |
| CCTA | 7.29E−04 | 1.87E−01 | −0.692 | 29 | −0.446 |
| TCGC | 7.59E−04 | 1.94E−01 | 0.690 | 30 | 0.600 |
| TTGG | 8.30E−04 | 2.12E−01 | 0.686 | 31 | 0.591 |
| GTGC | 8.62E−04 | 2.21E−01 | 0.685 | 32 | 0.354 |
| TCGG | 8.96E−04 | 2.30E−01 | 0.683 | 33 | 0.586 |
| CCAT | 9.36E−04 | 2.40E−01 | −0.682 | 34 | −0.159 |
| CAGT | 9.81E−04 | 2.51E−01 | 0.680 | 35 | 0.336 |
| TATC | 9.88E−04 | 2.53E−01 | −0.679 | 36 | −0.477 |
| GACC | 1.06E−03 | 2.72E−01 | −0.676 | 37 | −0.609 |
| GCGG | 1.10E−03 | 2.81E−01 | 0.675 | 38 | 0.444 |
| TGGG | 1.10E−03 | 2.82E−01 | 0.675 | 39 | 0.482 |
| ACGT | 1.15E−03 | 2.95E−01 | 0.673 | 40 | 0.300 |
| CCCT | 1.19E−03 | 3.05E−01 | −0.671 | 41 | −0.102 |
| TGGT | 1.21E−03 | 3.09E−01 | 0.671 | 42 | 0.426 |
| ATGG | 1.46E−03 | 3.73E−01 | 0.663 | 43 | 0.388 |
| ATGC | 1.49E−03 | 3.80E−01 | 0.662 | 44 | 0.439 |
| TCTA | 1.73E−03 | 4.43E−01 | −0.655 | 45 | −0.372 |
| TTCG | 1.73E−03 | 4.44E−01 | 0.655 | 46 | 0.523 |
| TCGT | 1.76E−03 | 4.51E−01 | 0.654 | 47 | 0.547 |
| AGGA | 2.01E−03 | 5.14E−01 | 0.648 | 48 | 0.415 |
| CGAT | 2.07E−03 | 5.31E−01 | −0.646 | 49 | −0.181 |
| CTGG | 2.28E−03 | 5.84E−01 | 0.642 | 50 | 0.461 |
| GCGC | 2.40E−03 | 6.13E−01 | 0.640 | 51 | 0.404 |
| CCCA | 2.44E−03 | 6.25E−01 | −0.639 | 52 | −0.056 |
| GTCG | 2.50E−03 | 6.41E−01 | 0.637 | 53 | 0.103 |
| GCCT | 2.68E−03 | 6.87E−01 | −0.634 | 54 | −0.541 |
| TAGG | 2.79E−03 | 7.15E−01 | 0.632 | 55 | 0.391 |
| GAGG | 2.84E−03 | 7.26E−01 | 0.631 | 56 | 0.206 |
| TCGA | 3.01E−03 | 7.70E−01 | 0.628 | 57 | 0.560 |
| TTGA | 3.37E−03 | 8.63E−01 | 0.623 | 58 | 0.494 |
| CCGG | 3.53E−03 | 9.02E−01 | 0.620 | 59 | 0.477 |
| TATA | 3.54E−03 | 9.06E−01 | −0.620 | 60 | −0.374 |
| CTGC | 3.54E−03 | 9.06E−01 | 0.620 | 61 | 0.468 |
| AGGG | 4.01E−03 | 1.00E+00 | 0.614 | 62 | 0.377 |
| TAGA | 4.18E−03 | 1.00E+00 | 0.611 | 63 | 0.382 |
| ATCG | 4.42E−03 | 1.00E+00 | 0.608 | 64 | 0.145 |
| CTGT | 4.51E−03 | 1.00E+00 | 0.607 | 65 | 0.454 |
| CCCC | 4.79E−03 | 1.00E+00 | −0.604 | 66 | −0.024 |
| CGTT | 4.89E−03 | 1.00E+00 | −0.603 | 67 | −0.203 |
| TAGC | 5.14E−03 | 1.00E+00 | 0.600 | 68 | 0.343 |
| CATT | 5.49E−03 | 1.00E+00 | −0.597 | 69 | −0.348 |
| CACT | 5.56E−03 | 1.00E+00 | −0.596 | 70 | −0.115 |
| GCCA | 5.77E−03 | 1.00E+00 | −0.594 | 71 | −0.497 |
| ACCT | 6.21E−03 | 1.00E+00 | −0.590 | 72 | −0.421 |
| AGAC | 6.31E−03 | 1.00E+00 | 0.589 | 73 | 0.328 |
| ACAG | 6.55E−03 | 1.00E+00 | −0.587 | 74 | −0.471 |
| ACCC | 7.24E−03 | 1.00E+00 | −0.581 | 75 | −0.459 |
| TGTA | 7.28E−03 | 1.00E+00 | −0.581 | 76 | −0.223 |
| CATC | 7.46E−03 | 1.00E+00 | −0.579 | 77 | −0.342 |
| CGTG | 7.48E−03 | 1.00E+00 | −0.579 | 78 | −0.239 |
| GAGA | 8.30E−03 | 1.00E+00 | 0.573 | 79 | 0.157 |
| CTAG | 9.16E−03 | 1.00E+00 | 0.567 | 80 | 0.464 |
| TATT | 1.02E−02 | 1.00E+00 | −0.560 | 81 | −0.330 |
| CCGC | 1.26E−02 | 1.00E+00 | 0.547 | 82 | 0.440 |
| ACAT | 1.39E−02 | 1.00E+00 | −0.540 | 83 | −0.391 |
| GAGC | 1.42E−02 | 1.00E+00 | 0.539 | 84 | 0.158 |
| CTAC | 1.50E−02 | 1.00E+00 | 0.535 | 85 | 0.416 |
| GCAT | 1.56E−02 | 1.00E+00 | −0.533 | 86 | −0.414 |
| GACT | 1.60E−02 | 1.00E+00 | −0.531 | 87 | −0.432 |
| GCTT | 1.65E−02 | 1.00E+00 | −0.529 | 88 | −0.428 |
| CATA | 1.83E−02 | 1.00E+00 | 0.522 | 89 | −0.387 |
| ACTG | 1.91E−02 | 1.00E+00 | −0.519 | 90 | −0.399 |
| GTAG | 1.93E−02 | 1.00E+00 | 0.518 | 91 | 0.201 |
| GCTA | 1.97E−02 | 1.00E+00 | −0.517 | 92 | −0.452 |
| CTTA | 2.01E−02 | 1.00E+00 | −0.515 | 93 | −0.080 |
| ATGA | 2.14E−02 | 1.00E+00 | 0.511 | 94 | 0.255 |
| GCCC | 2.15E−02 | 1.00E+00 | −0.510 | 95 | −0.549 |
| AGAA | 2.30E−02 | 1.00E+00 | 0.505 | 96 | 0.276 |
| ACTT | 2.41E−02 | 1.00E+00 | −0.502 | 97 | −0.370 |
| ACTA | 2.47E−02 | 1.00E+00 | −0.500 | 98 | −0.363 |
| ATGT | 2.54E−02 | 1.00E+00 | 0.498 | 99 | 0.229 |
| AAGC | 2.73E−02 | 1.00E+00 | 0.493 | 100 | 0.098 |
| GCAA | 2.74E−02 | 1.00E+00 | −0.492 | 101 | −0.321 |
| AAGT | 2.80E−02 | 1.00E+00 | 0.491 | 102 | −0.022 |
| CCGA | 3.01E−02 | 1.00E+00 | 0.485 | 103 | 0.470 |
| CGTC | 3.13E−02 | 1.00E+00 | −0.482 | 104 | −0.162 |
| CATG | 3.15E−02 | 1.00E+00 | −0.482 | 105 | −0.414 |
| GTGA | 3.34E−02 | 1.00E+00 | 0.477 | 106 | 0.115 |
| GCTG | 3.36E−02 | 1.00E+00 | −0.477 | 107 | −0.450 |
| CTCA | 3.47E−02 | 1.00E+00 | 0.474 | 108 | 0.338 |
| ATAC | 3.47E−02 | 1.00E+00 | 0.474 | 109 | 0.217 |
| GGGG | 3.62E−02 | 1.00E+00 | 0.471 | 110 | 0.106 |
| TAGT | 3.68E−02 | 1.00E+00 | 0.469 | 111 | 0.251 |
| ACTC | 3.78E−02 | 1.00E+00 | −0.467 | 112 | −0.369 |
| CCAA | 3.80E−02 | 1.00E+00 | −0.467 | 113 | 0.009 |
| CTCG | 3.82E−02 | 1.00E+00 | 0.466 | 114 | 0.326 |
| TTTG | 4.00E−02 | 1.00E+00 | 0.463 | 115 | 0.357 |
| GCTC | 4.11E−02 | 1.00E+00 | −0.460 | 116 | −0.439 |
| ACCG | 4.14E−02 | 1.00E+00 | −0.460 | 117 | −0.361 |
| GAGT | 4.16E−02 | 1.00E+00 | 0.459 | 118 | 0.015 |
| CTCC | 4.38E−02 | 1.00E+00 | 0.455 | 119 | 0.319 |
| CGGG | 4.41E−02 | 1.00E+00 | 0.454 | 120 | 0.274 |
| AACC | 4.59E−02 | 1.00E+00 | −0.451 | 121 | −0.439 |
| GGGC | 5.21E−02 | 1.00E+00 | 0.440 | 122 | 0.032 |
| CTCT | 5.36E−02 | 1.00E+00 | 0.438 | 123 | 0.308 |
| AAGA | 5.53E−02 | 1.00E+00 | 0.435 | 124 | 0.087 |
| GCAC | 5.63E−02 | 1.00E+00 | −0.433 | 125 | −0.344 |
| ATAG | 5.81E−02 | 1.00E+00 | 0.431 | 126 | 0.210 |
| ACAA | 6.13E−02 | 1.00E+00 | −0.426 | 127 | −0.319 |
| GATC | 6.26E−02 | 1.00E+00 | −0.424 | 128 | −0.412 |
| AGAG | 6.59E−02 | 1.00E+00 | 0.419 | 129 | 0.178 |
| TGAA | 6.71E−02 | 1.00E+00 | 0.417 | 130 | 0.419 |
| TATG | 6.80E−02 | 1.00E+00 | −0.416 | 131 | −0.301 |
| AAGG | 6.87E−02 | 1.00E+00 | 0.415 | 132 | 0.008 |
| CCAG | 7.14E−02 | 1.00E+00 | −0.412 | 133 | −0.014 |
| TITC | 7.19E−02 | 1.00E+00 | 0.411 | 134 | 0.311 |
| ACCA | 7.75E−02 | 1.00E+00 | −0.404 | 135 | −0.334 |
| GGCT | 7.85E−02 | 1.00E+00 | −0.402 | 136 | −0.375 |
| CACA | 8.15E−02 | 1.00E+00 | −0.399 | 137 | 0.086 |
| GTTA | 8.38E−02 | 1.00E+00 | −0.396 | 138 | −0.387 |
| CCAC | 8.85E−02 | 1.00E+00 | −0.391 | 139 | 0.029 |
| GATA | 8.87E−02 | 1.00E+00 | −0.391 | 140 | −0.353 |
| CGGA | 9.01E−02 | 1.00E+00 | 0.389 | 141 | 0.304 |
| ACAC | 9.09E−02 | 1.00E+00 | −0.388 | 142 | −0.393 |
| GTGT | 9.41E−02 | 1.00E+00 | 0.385 | 143 | 0.054 |
| GGGA | 9.47E−02 | 1.00E+00 | 0.384 | 144 | 0.004 |
| GTTT | 9.71E−02 | 1.00E+00 | −0.381 | 145 | −0.372 |
| TAAG | 9.71E−02 | 1.00E+00 | 0.381 | 146 | 0.272 |
| GGCC | 9.79E−02 | 1.00E+00 | −0.381 | 147 | −0.437 |
| TGAG | 1.07E−01 | 1.00E+00 | 0.371 | 148 | 0.306 |
| TGAC | 1.08E−01 | 1.00E+00 | 0.370 | 149 | 0.321 |
| CAAT | 1.09E−01 | 1.00E+00 | −0.369 | 150 | 0.087 |
| CGGT | 1.09E−01 | 1.00E+00 | 0.369 | 151 | 0.131 |
| GACA | 1.14E−01 | 1.00E+00 | −0.365 | 152 | −0.340 |
| TGTT | 1.14E−01 | 1.00E+00 | −0.365 | 153 | −0.062 |
| TCTT | 1.15E−01 | 1.00E+00 | 0.364 | 154 | −0.210 |
| CACC | 1.18E−01 | 1.00E+00 | −0.361 | 155 | 0.005 |
| GGTT | 1.19E−01 | 1.00E+00 | −0.360 | 156 | −0.328 |
| TGAT | 1.25E−01 | 1.00E+00 | 0.354 | 157 | 0.414 |
| GAAG | 1.32E−01 | 1.00E+00 | 0.349 | 158 | −0.043 |
| GTTC | 1.39E−01 | 1.00E+00 | −0.343 | 159 | −0.391 |
| TCCG | 1.46E−01 | 1.00E+00 | 0.337 | 160 | 0.262 |
| GGTA | 1.47E−01 | 1.00E+00 | −0.336 | 161 | −0.310 |
| GATT | 1.48E−01 | 1.00E+00 | −0.336 | 162 | −0.347 |
| GTAC | 1.50E−01 | 1.00E+00 | 0.334 | 163 | 0.080 |
| AATA | 1.65E−01 | 1.00E+00 | −0.323 | 164 | −0.343 |
| ATCA | 1.70E−01 | 1.00E+00 | 0.319 | 165 | 0.007 |
| GGGT | 1.71E−01 | 1.00E+00 | 0.319 | 166 | −0.063 |
| ATCC | 1.72E−01 | 1.00E+00 | 0.318 | 167 | −0.024 |
| AGCC | 1.74E−01 | 1.00E+00 | 0.316 | 168 | 0.014 |
| GATG | 1.82E−01 | 1.00E+00 | −0.311 | 169 | −0.386 |
| AATG | 1.83E−01 | 1.00E+00 | −0.310 | 170 | −0.382 |
| AACT | 1.84E−01 | 1.00E+00 | −0.310 | 171 | −0.342 |
| CTTT | 1.87E−01 | 1.00E+00 | −0.307 | 172 | 0.110 |
| AGAT | 1.94E−01 | 1.00E+00 | 0.303 | 173 | −0.002 |
| TCTC | 1.99E−01 | 1.00E+00 | −0.300 | 174 | −0.211 |
| GTCC | 2.00E−01 | 1.00E+00 | 0.299 | 175 | −0.031 |
| CGCA | 2.04E−01 | 1.00E+00 | −0.297 | 176 | −0.059 |
| TAAT | 2.08E−01 | 1.00E+00 | 0.294 | 177 | 0.113 |
| GCCG | 2.15E−01 | 1.00E+00 | −0.290 | 178 | −0.352 |
| GGTC | 2.18E−01 | 1.00E+00 | −0.288 | 179 | −0.390 |
| TACT | 2.26E−01 | 1.00E+00 | −0.284 | 180 | −0.152 |
| CCCG | 2.33E−01 | 1.00E+00 | −0.279 | 181 | 0.069 |
| CGCT | 2.35E−01 | 1.00E+00 | −0.278 | 182 | −0.067 |
| CTGA | 2.39E−01 | 1.00E+00 | 0.276 | 183 | 0.355 |
| GCAG | 2.44E−01 | 1.00E+00 | −0.273 | 184 | −0.208 |
| TGTG | 2.48E−01 | 1.00E+00 | −0.271 | 185 | −0.150 |
| AGCA | 2.72E−01 | 1.00E+00 | 0.258 | 186 | 0.026 |
| ATAA | 2.80E−01 | 1.00E+00 | 0.254 | 187 | 0.080 |
| AACG | 2.84E−01 | 1.00E+00 | 0.252 | 188 | 0.240 |
| AATC | 2.88E−01 | 1.00E+00 | −0.250 | 189 | −0.346 |
| TAAA | 2.99E−01 | 1.00E+00 | 0.244 | 190 | 0.247 |
| TACC | 3.19E−01 | 1.00E+00 | −0.235 | 191 | −0.148 |
| ATAT | 3.22E−01 | 1.00E+00 | 0.233 | 192 | −0.007 |
| AAAA | 3.28E−01 | 1.00E+00 | 0.231 | 193 | −0.010 |
| CGAG | 3.36E−01 | 1.00E+00 | −0.227 | 194 | 0.019 |
| CTAA | 3.43E−01 | 1.00E+00 | 0.224 | 195 | 0.372 |
| TACA | 3.50E−01 | 1.00E+00 | −0.221 | 196 | 0.094 |
| CGAA | 3.51E−01 | 1.00E+00 | −0.220 | 197 | 0.063 |
| CTAT | 3.52E−01 | 1.00E+00 | 0.220 | 198 | 0.307 |
| GGTG | 3.53E−01 | 1.00E+00 | −0.219 | 199 | −0.379 |
| GTAA | 3.54E−01 | 1.00E+00 | 0.219 | 200 | 0.005 |
| CGGC | 3.57E−01 | 1.00E+00 | 0.217 | 201 | 0.121 |
| AGCT | 3.57E−01 | 1.00E+00 | 0.217 | 202 | −0.083 |
| GTCA | 3.69E−01 | 1.00E+00 | 0.212 | 203 | −0.036 |
| TTTT | 3.72E−01 | 1.00E+00 | 0.211 | 204 | 0.217 |
| AGTC | 3.80E−01 | 1.00E+00 | 0.207 | 205 | −0.112 |
| GAAA | 3.84E−01 | 1.00E+00 | 0.206 | 206 | −0.064 |
| ATCT | 3.86E−01 | 1.00E+00 | 0.205 | 207 | −0.117 |
| TGTC | 3.92E−01 | 1.00E+00 | −0.202 | 208 | −0.055 |
| AACA | 3.96E−01 | 1.00E+00 | −0.201 | 209 | −0.317 |
| TCAG | 3.97E−01 | 1.00E+00 | 0.200 | 210 | 0.192 |
| GGAC | 4.05E−01 | 1.00E+00 | 0.197 | 211 | −0.167 |
| CACG | 4.35E−01 | 1.00E+00 | 0.185 | 212 | 0.205 |
| GGAT | 4.36E−01 | 1.00E+00 | −0.185 | 213 | −0.283 |
| ATTA | 4.43E−01 | 1.00E+00 | −0.182 | 214 | −0.310 |
| CGCC | 4.53E−01 | 1.00E+00 | −0.178 | 215 | −0.031 |
| TAAC | 4.61E−01 | 1.00E+00 | 0.175 | 216 | 0.151 |
| CTTC | 4.69E−01 | 1.00E+00 | −0.172 | 217 | 0.120 |
| TTTA | 4.74E−01 | 1.00E+00 | 0.170 | 218 | 0.220 |
| GGAG | 4.91E−01 | 1.00E+00 | 0.164 | 219 | −0.182 |
| AGTT | 5.21E−01 | 1.00E+00 | 0.153 | 220 | −0.166 |
| GTTG | 5.22E−01 | 1.00E+00 | −0.152 | 221 | −0.332 |
| TACG | 5.37E−01 | 1.00E+00 | 0.147 | 222 | 0.107 |
| CGAC | 5.38E−01 | 1.00E+00 | −0.147 | 223 | 0.020 |
| TCAA | 5.40E−01 | 1.00E+00 | 0.146 | 224 | 0.165 |
| TGCG | 5.75E−01 | 1.00E+00 | 0.134 | 225 | 0.081 |
| CGCG | 5.76E−01 | 1.00E+00 | 0.133 | 226 | 0.066 |
| GGCA | 5.81E−01 | 1.00E+00 | −0.131 | 227 | −0.129 |
| GGCG | 5.87E−01 | 1.00E+00 | 0.129 | 228 | −0.007 |
| GAAT | 6.08E−01 | 1.00E+00 | −0.122 | 229 | 0.256 |
| AGTA | 6.21E−01 | 1.00E+00 | −0.118 | 230 | −0.258 |
| AAAG | 6.28E−01 | 1.00E+00 | 0.115 | 231 | −0.134 |
| AATT | 6.36E−01 | 1.00E+00 | −0.113 | 232 | −0.282 |
| TCAC | 6.37E−01 | 1.00E+00 | 0.112 | 233 | 0.105 |
| ATTT | 6.83E−01 | 1.00E+00 | −0.097 | 234 | 0.262 |
| GACG | 6.87E−01 | 1.00E+00 | 0.096 | 235 | −0.143 |
| TGCA | 7.34E−01 | 1.00E+00 | −0.081 | 236 | 0.025 |
| TCCC | 7.44E−01 | 1.00E+00 | 0.078 | 237 | 0.064 |
| ATTC | 7.49E−01 | 1.00E+00 | −0.076 | 238 | −0.251 |
| TCTG | 7.52E−01 | 1.00E+00 | −0.076 | 239 | −0.053 |
| GAAC | 7.52E−01 | 1.00E+00 | 0.076 | 240 | −0.221 |
| TGCT | 7.53E−01 | 1.00E+00 | −0.075 | 241 | 0.027 |
| TGCC | 7.63E−01 | 1.00E+00 | 0.072 | 242 | 0.084 |
| CAAC | 8.12E−01 | 1.00E+00 | −0.057 | 243 | 0.208 |
| TCCT | 8.51E−01 | 1.00E+00 | −0.045 | 244 | 0.025 |
| TCCA | 8.53E−01 | 1.00E+00 | 0.044 | 245 | 0.081 |
| GTAT | 8.54E−01 | 1.00E+00 | 0.044 | 246 | −0.102 |
| GGAA | 8.54E−01 | 1.00E+00 | 0.044 | 247 | −0.157 |
| CTTG | 8.58E−01 | 1.00E+00 | −0.043 | 248 | 0.201 |
| ATTG | 8.68E−01 | 1.00E+00 | 0.040 | 249 | −0.183 |
| CAAG | 8.69E−01 | 1.00E+00 | −0.039 | 250 | 0.233 |
| AAAC | 9.47E−01 | 1.00E+00 | −0.016 | 251 | −0.213 |
| AAAT | 9.63E−01 | 1.00E+00 | 0.011 | 252 | −0.189 |
| TCAT | 9.69E−01 | 1.00E+00 | −0.009 | 253 | 0.027 |
| GTCT | 9.79E−01 | 1.00E+00 | 0.006 | 254 | −0.172 |
| AGTG | 9.86E−01 | 1.00E+00 | 0.004 | 255 | −0.215 |
| CAAA | 9.93E−01 | 1.00E+00 | −0.002 | 256 | 0.279 |

### C. Ranked End Motifs for Fetal Fraction

Table 3 shows correlation between 4-mer end motifs and fetal fraction from a cohort of pregnant individuals, including first, second and third trimester, with the cohort being split 50% into training set and 50% into a testing set (total of n=60 subjects). Bonferroni's correction was used to adjust the p-value for multiple comparisons. End motifs were ranked according to adjusted P-value from the training set. A set of one or more sequence motifs may be selected from the top 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 160 sequence motifs listed in Table 3 for lowest p-value (an adjusted p-value as ranked below) or highest absolute Pearson r value as determined for the training set or the testing set. In addition to the listed 4-mers, other end motifs of other lengths may be used, such as 3-mers related to 4-mers listed. For example, GCAA, GCAC, GCAG, and GCAT all appear in the top 80 end motifs of Table 1, and thus using an mount of GCA is related to the these 4-mers. A 3-mer could be used as long as at least one, two, or three of the related 4-mers are present. The same goes for other tables herein.

In some embodiments, the set of one or more sequence motifs can be selected based on a threshold p-value (e.g., adjusted p-value) or a Pearson r. For example, all end motifs above a specified Pearson r can be selected or all end motifs below a specific p-value can be selected. Example threshold Pearson r values include 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50, 0.49, 0.48, 0.47, 0.46, 0.45, 0.44, 0.43, 0.42, or 0.41.

| | | Adjusted | | | |
|---|---|---|---|---|---|
| 5′ End | | P-value | Pearson r | Ranking | Pearson r |
| Motif | P-value | (Bonferroni) | (Training Set) | (Training Set) | (Testing Set) |
| CGAT | 2.22E−05 | 5.68E−03 | −0.693 | 1 | −0.564 |
| ACGT | 1.73E−04 | 4.43E−02 | 0.633 | 2 | 0.728 |
| GGGT | 8.12E−04 | 2.08E−01 | −0.579 | 3 | −0.667 |
| AAAA | 9.89E−04 | 2.53E−01 | 0.571 | 4 | 0.728 |
| CAAA | 1.02E−03 | 2.60E−01 | 0.570 | 5 | 0.691 |
| AGTG | 1.14E−03 | 2.93E−01 | −0.565 | 6 | −0.710 |
| GGTC | 1.35E−03 | 3.45E−01 | −0.558 | 7 | −0.794 |
| GGGG | 1.43E−03 | 3.65E−01 | −0.556 | 8 | −0.638 |
| GGAG | 1.47E−03 | 3.76E−01 | −0.555 | 9 | −0.675 |
| ACGA | 1.52E−03 | 3.89E−01 | 0.553 | 10 | 0.749 |
| GGGC | 1.73E−03 | 4.43E−01 | −0.548 | 11 | −0.630 |
| GGGA | 1.77E−03 | 4.53E−01 | −0.547 | 12 | −0.686 |
| CGTG | 1.85E−03 | 4.74E−01 | −0.545 | 13 | −0.653 |
| CGAC | 1.97E−03 | 5.05E−01 | −0.542 | 14 | −0.565 |
| GAGG | 2.02E−03 | 5.17E−01 | −0.541 | 15 | −0.388 |
| CGGT | 2.15E−03 | 5.50E−01 | −0.538 | 16 | −0.550 |
| GGTG | 2.24E−03 | 5.75E−01 | −0.536 | 17 | −0.791 |
| GGAC | 2.33E−03 | 5.98E−01 | −0.535 | 18 | −0.735 |
| CCTA | 2.41E−03 | 6.18E−01 | 0.533 | 19 | −0.006 |
| ATCC | 2.51E−03 | 6.43E−01 | −0.531 | 20 | −0.531 |
| CGAG | 2.61E−03 | 6.68E−01 | −0.530 | 21 | −0.585 |
| CGAA | 2.61E−03 | 6.69E−01 | −0.530 | 22 | −0.296 |
| GTCG | 2.77E−03 | 7.09E−01 | −0.527 | 23 | −0.681 |
| GAGC | 2.93E−03 | 7.49E−01 | −0.524 | 24 | −0.293 |
| AGGG | 3.02E−03 | 7.74E−01 | −0.523 | 25 | −0.314 |
| ACAA | 3.03E−03 | 7.76E−01 | 0.523 | 26 | 0.447 |
| CCAT | 3.19E−03 | 8.16E−01 | 0.521 | 27 | −0.087 |
| CGGA | 3.25E−03 | 8.31E−01 | −0.520 | 28 | −0.521 |
| AGCC | 3.30E−03 | 8.46E−01 | −0.519 | 29 | −0.598 |
| AGCG | 3.33E−03 | 8.52E−01 | −0.519 | 30 | −0.631 |
| CATA | 3.34E−03 | 8.55E−01 | 0.518 | 31 | 0.447 |
| AGAC | 3.47E−03 | 8.88E−01 | −0.517 | 32 | −0.059 |
| CATT | 3.47E−03 | 8.89E−01 | 0.517 | 33 | 0.439 |
| GGCT | 3.49E−03 | 8.92E−01 | −0.516 | 34 | −0.778 |
| CAAT | 3.52E−03 | 9.00E−01 | 0.516 | 35 | 0.568 |
| CGGG | 3.63E−03 | 9.29E−01 | −0.514 | 36 | −0.547 |
| CCAA | 3.68E−03 | 9.43E−01 | 0.514 | 37 | −0.009 |
| AGGC | 4.03E−03 | 1.00E+00 | −0.509 | 38 | −0.304 |
| CGGC | 4.33E−03 | 1.00E+00 | −0.506 | 39 | −0.579 |
| GAAA | 4.35E−03 | 1.00E+00 | 0.506 | 40 | 0.621 |
| GCTA | 4.43E−03 | 1.00E+00 | 0.505 | 41 | 0.231 |
| TCCC | 4.52E−03 | 1.00E+00 | −0.504 | 42 | −0.525 |
| GTGG | 4.57E−03 | 1.00E+00 | −0.503 | 43 | −0.099 |
| GCAA | 4.63E−03 | 1.00E+00 | 0.503 | 44 | 0.467 |
| GGCC | 4.81E−03 | 1.00E+00 | −0.501 | 45 | −0.687 |
| GGCG | 4.84E−03 | 1.00E+00 | −0.501 | 46 | −0.705 |
| CTCC | 4.90E−03 | 1.00E+00 | −0.500 | 47 | −0.533 |
| CGTC | 4.99E−03 | 1.00E+00 | −0.499 | 48 | −0.700 |
| TAAA | 5.00E−03 | 1.00E+00 | 0.499 | 49 | 0.759 |
| AAAT | 5.04E−03 | 1.00E+00 | 0.499 | 50 | 0.616 |
| CGCA | 5.16E−03 | 1.00E+00 | −0.497 | 51 | −0.657 |
| GTGC | 5.18E−03 | 1.00E+00 | −0.497 | 52 | −0.193 |
| TGAA | 5.48E−03 | 1.00E+00 | 0.494 | 53 | 0.758 |
| ACCG | 5.72E−03 | 1.00E+00 | −0.492 | 54 | −0.802 |
| AGAG | 6.24E−03 | 1.00E+00 | −0.488 | 55 | −0.102 |
| TATT | 6.30E−03 | 1.00E+00 | 0.487 | 56 | 0.577 |
| TGTA | 6.35E−03 | 1.00E+00 | 0.487 | 57 | 0.561 |
| TGAT | 6.42E−03 | 1.00E+00 | 0.486 | 58 | 0.722 |
| TGTT | 6.68E−03 | 1.00E+00 | 0.484 | 59 | 0.600 |
| CGCG | 6.70E−03 | 1.00E+00 | −0.484 | 60 | −0.568 |
| AGTC | 6.71E−03 | 1.00E+00 | −0.484 | 61 | −0.675 |
| CTTA | 6.83E−03 | 1.00E+00 | 0.483 | 62 | 0.475 |
| AATA | 7.11E−03 | 1.00E+00 | 0.481 | 63 | 0.484 |
| GACG | 7.49E−03 | 1.00E+00 | −0.478 | 64 | −0.706 |
| CGCT | 7.53E−03 | 1.00E+00 | −0.478 | 65 | −0.667 |
| CGCC | 7.89E−03 | 1.00E+00 | −0.476 | 66 | −0.610 |
| AGCT | 7.93E−03 | 1.00E+00 | −0.475 | 67 | −0.737 |
| AGAA | 8.23E−03 | 1.00E+00 | 0.473 | 68 | 0.755 |
| AGGT | 8.30E−03 | 1.00E+00 | −0.473 | 69 | 0.057 |
| CTCT | 8.60E−03 | 1.00E+00 | −0.471 | 70 | −0.433 |
| ATAA | 8.98E−03 | 1.00E+00 | 0.469 | 71 | 0.691 |
| TGGG | 9.02E−03 | 1.00E+00 | −0.468 | 72 | −0.252 |
| AATT | 9.04E−03 | 1.00E+00 | 0.468 | 73 | 0.461 |
| AGCA | 9.41E−03 | 1.00E+00 | −0.466 | 74 | −0.663 |
| TCTA | 9.47E−03 | 1.00E+00 | 0.466 | 75 | 0.490 |
| AAGA | 9.48E−03 | 1.00E+00 | 0.466 | 76 | 0.811 |
| GACC | 9.53E−03 | 1.00E+00 | −0.466 | 77 | −0.734 |
| TCTC | 9.95E−03 | 1.00E+00 | −0.463 | 78 | −0.544 |
| TATA | 1.01E−02 | 1.00E+00 | 0.463 | 79 | 0.525 |
| TGGC | 1.06E−02 | 1.00E+00 | −0.460 | 80 | −0.263 |
| CTTT | 1.10E−02 | 1.00E+00 | 0.458 | 81 | 0.427 |
| CTCG | 1.15E−02 | 1.00E+00 | −0.455 | 82 | −0.417 |
| TCCT | 1.16E−02 | 1.00E+00 | −0.455 | 83 | −0.488 |
| CAGC | 1.17E−02 | 1.00E+00 | −0.454 | 84 | −0.433 |
| TAAT | 1.23E−02 | 1.00E+00 | 0.452 | 85 | 0.683 |
| ACCC | 1.31E−02 | 1.00E+00 | −0.448 | 86 | −0.726 |
| CTAA | 1.34E−02 | 1.00E+00 | 0.447 | 87 | 0.670 |
| GCGC | 1.41E−02 | 1.00E+00 | −0.444 | 88 | −0.477 |
| TCAA | 1.46E−02 | 1.00E+00 | 0.442 | 89 | 0.748 |
| CAGG | 1.50E−02 | 1.00E+00 | −0.440 | 90 | −0.349 |
| TCCG | 1.53E−02 | 1.00E+00 | −0.439 | 91 | −0.350 |
| ACTA | 1.53E−02 | 1.00E+00 | 0.439 | 92 | 0.184 |
| CCCG | 1.67E−02 | 1.00E+00 | −0.433 | 93 | −0.631 |
| CCTC | 1.68E−02 | 1.00E+00 | −0.433 | 94 | −0.712 |
| TCAT | 1.72E−02 | 1.00E+00 | 0.432 | 95 | 0.640 |
| ATTT | 1.77E−02 | 1.00E+00 | 0.430 | 96 | 0.412 |
| AACA | 1.79E−02 | 1.00E+00 | 0.429 | 97 | 0.425 |
| CACG | 1.82E−02 | 1.00E+00 | −0.428 | 98 | −0.630 |
| TCGT | 1.89E−02 | 1.00E+00 | 0.426 | 99 | 0.682 |
| GCAT | 1.92E−02 | 1.00E+00 | 0.425 | 100 | 0.248 |
| CCCC | 1.92E−02 | 1.00E+00 | −0.425 | 101 | −0.662 |
| GCCG | 1.94E−02 | 1.00E+00 | −0.424 | 102 | −0.647 |
| TGAG | 2.04E−02 | 1.00E+00 | −0.421 | 103 | −0.075 |
| GGAT | 2.05E−02 | 1.00E+00 | −0.421 | 104 | −0.767 |
| GTTT | 2.05E−02 | 1.00E+00 | 0.421 | 105 | 0.351 |
| GTTA | 2.10E−02 | 1.00E+00 | 0.420 | 106 | 0.369 |
| AAGG | 2.12E−02 | 1.00E+00 | −0.419 | 107 | 0.218 |
| CTAT | 2.13E−02 | 1.00E+00 | 0.419 | 108 | 0.620 |
| CTGC | 2.15E−02 | 1.00E+00 | 0.418 | 109 | −0.178 |
| ATAT | 2.23E−02 | 1.00E+00 | 0.416 | 110 | 0.587 |
| AAGC | 2.28E−02 | 1.00E+00 | −0.414 | 111 | 0.160 |
| GAAT | 2.32E−02 | 1.00E+00 | 0.413 | 112 | 0.439 |
| ATCG | 2.33E−02 | 1.00E+00 | −0.413 | 113 | −0.307 |
| ATTA | 2.38E−02 | 1.00E+00 | 0.412 | 114 | 0.360 |
| ACAT | 2.39E−02 | 1.00E+00 | 0.411 | 115 | 0.106 |
| TAGG | 2.53E−02 | 1.00E+00 | −0.408 | 116 | 0.168 |
| TCTT | 2.71E−02 | 1.00E+00 | 0.403 | 117 | 0.407 |
| GATA | 2.71E−02 | 1.00E+00 | 0.403 | 118 | 0.304 |
| GCGG | 2.73E−02 | 1.00E+00 | −0.403 | 119 | −0.320 |
| TTTA | 2.74E−02 | 1.00E+00 | 0.403 | 120 | 0.689 |
| CCGC | 2.74E−02 | 1.00E+00 | −0.403 | 121 | −0.480 |
| TCAC | 2.79E−02 | 1.00E+00 | −0.401 | 122 | −0.248 |
| GATT | 2.81E−02 | 1.00E+00 | 0.401 | 123 | 0.320 |
| GTAA | 2.90E−02 | 1.00E+00 | 0.399 | 124 | 0.585 |
| GCTT | 3.35E−02 | 1.00E+00 | 0.389 | 125 | 0.125 |
| AGAT | 3.37E−02 | 1.00E+00 | 0.389 | 126 | 0.607 |
| ACGC | 3.41E−02 | 1.00E+00 | −0.388 | 127 | −0.387 |
| TTGA | 3.49E−02 | 1.00E+00 | 0.386 | 128 | 0.775 |
| CTGG | 3.66E−02 | 1.00E+00 | −0.383 | 129 | −0.010 |
| GGCA | 3.67E−02 | 1.00E+00 | −0.383 | 130 | −0.709 |
| CCTG | 3.72E−02 | 1.00E+00 | −0.382 | 131 | −0.672 |
| AGGA | 3.79E−02 | 1.00E+00 | −0.381 | 132 | 0.217 |
| AACT | 3.85E−02 | 1.00E+00 | 0.380 | 133 | 0.313 |
| AACG | 3.94E−02 | 1.00E+00 | −0.378 | 134 | −0.579 |
| GTAT | 4.28E−02 | 1.00E+00 | 0.372 | 135 | 0.420 |
| CCAG | 4.49E−02 | 1.00E+00 | −0.369 | 136 | −0.611 |
| CCGG | 4.51E−02 | 1.00E+00 | −0.368 | 137 | −0.417 |
| TTCC | 4.53E−02 | 1.00E+00 | −0.368 | 138 | 0.462 |
| TGCG | 4.81E−02 | 1.00E+00 | −0.364 | 139 | −0.312 |
| GCCC | 4.86E−02 | 1.00E+00 | −0.363 | 140 | −0.621 |
| CCTT | 5.85E−02 | 1.00E+00 | 0.349 | 141 | −0.448 |
| CGTT | 6.04E−02 | 1.00E+00 | −0.347 | 142 | −0.284 |
| AATG | 6.08E−02 | 1.00E+00 | 0.346 | 143 | 0.301 |
| CTCA | 6.18E−02 | 1.00E+00 | −0.345 | 144 | −0.199 |
| CCAC | 6.32E−02 | 1.00E+00 | −0.343 | 145 | −0.644 |
| ATGA | 6.33E−02 | 1.00E+00 | 0.343 | 146 | 0.716 |
| TACA | 6.36E−02 | 1.00E+00 | 0.343 | 147 | 0.523 |
| TAGC | 6.43E−02 | 1.00E+00 | −0.342 | 148 | 0.192 |
| ATTG | 6.51E−02 | 1.00E+00 | 0.341 | 149 | 0.390 |
| TATC | 6.93E−02 | 1.00E+00 | 0.336 | 150 | 0.379 |
| TGGT | 6.97E−02 | 1.00E+00 | −0.336 | 151 | 0.170 |
| TTAA | 7.07E−02 | 1.00E+00 | 0.335 | 152 | 0.759 |
| AGTT | 7.34E−02 | 1.00E+00 | 0.332 | 153 | 0.277 |
| CACT | 7.37E−02 | 1.00E+00 | 0.331 | 154 | −0.177 |
| TGCT | 7.41E−02 | 1.00E+00 | 0.331 | 155 | 0.377 |
| AAAC | 7.73E−02 | 1.00E+00 | 0.327 | 156 | 0.523 |
| CACC | 7.79E−02 | 1.00E+00 | −0.327 | 157 | −0.694 |
| AAAG | 7.97E−02 | 1.00E+00 | 0.325 | 158 | 0.772 |
| CCCA | 8.04E−02 | 1.00E+00 | −0.324 | 159 | −0.663 |
| AGTA | 8.20E−02 | 1.00E+00 | 0.323 | 160 | 0.175 |
| TATG | 8.40E−02 | 1.00E+00 | 0.321 | 161 | 0.453 |
| TACT | 8.48E−02 | 1.00E+00 | 0.320 | 162 | 0.472 |
| AAGT | 8.96E−02 | 1.00E+00 | 0.315 | 163 | 0.708 |
| GATC | 9.09E−02 | 1.00E+00 | −0.314 | 164 | −0.545 |
| TTTT | 9.56E−02 | 1.00E+00 | 0.310 | 165 | 0.687 |
| GCTC | 9.62E−02 | 1.00E+00 | −0.309 | 166 | −0.654 |
| ATGT | 1.03E−01 | 1.00E+00 | 0.303 | 167 | 0.645 |
| GAAG | 1.05E−01 | 1.00E+00 | −0.302 | 168 | −0.098 |
| ATGG | 1.07E−01 | 1.00E+00 | −0.300 | 169 | 0.452 |
| TCGA | 1.09E−01 | 1.00E+00 | 0.298 | 170 | 0.684 |
| GTTC | 1.11E−01 | 1.00E+00 | 0.297 | 171 | 0.199 |
| ACCT | 1.11E−01 | 1.00E+00 | −0.297 | 172 | −0.730 |
| GAGT | 1.13E−01 | 1.00E+00 | −0.296 | 173 | 0.091 |
| CTAC | 1.13E−01 | 1.00E+00 | −0.295 | 174 | 0.052 |
| GACT | 1.16E−01 | 1.00E+00 | 0.293 | 175 | 0.040 |
| ATTC | 1.19E−01 | 1.00E+00 | 0.291 | 176 | 0.260 |
| GTTG | 1.19E−01 | 1.00E+00 | 0.291 | 177 | 0.258 |
| AATC | 1.29E−01 | 1.00E+00 | 0.284 | 178 | 0.139 |
| TGGA | 1.29E−01 | 1.00E+00 | −0.284 | 179 | 0.305 |
| TGCC | 1.30E−01 | 1.00E+00 | −0.283 | 180 | −0.322 |
| ACGG | 1.36E−01 | 1.00E+00 | −0.278 | 181 | −0.126 |
| TCGC | 1.41E−01 | 1.00E+00 | −0.275 | 182 | −0.025 |
| TTAT | 1.42E−01 | 1.00E+00 | 0.275 | 183 | 0.727 |
| CGTA | 1.45E−01 | 1.00E+00 | 0.272 | 184 | 0.137 |
| ACTT | 1.86E−01 | 1.00E+00 | 0.248 | 185 | −0.050 |
| GTCC | 2.00E−01 | 1.00E+00 | −0.241 | 186 | −0.285 |
| TCGG | 2.01E−01 | 1.00E+00 | −0.240 | 187 | 0.199 |
| GCTG | 2.03E−01 | 1.00E+00 | −0.239 | 188 | −0.572 |
| GTCA | 2.05E−01 | 1.00E+00 | 0.238 | 189 | 0.232 |
| TACC | 2.14E−01 | 1.00E+00 | −0.234 | 190 | −0.193 |
| GCCT | 2.26E−01 | 1.00E+00 | −0.228 | 191 | −0.621 |
| GAAC | 2.29E−01 | 1.00E+00 | −0.226 | 192 | −0.388 |
| GTAC | 2.30E−01 | 1.00E+00 | 0.226 | 193 | 0.328 |
| TCCA | 2.34E−01 | 1.00E+00 | −0.224 | 194 | −0.071 |
| CCGA | 2.46E−01 | 1.00E+00 | −0.219 | 195 | −0.304 |
| TCAG | 2.51E−01 | 1.00E+00 | −0.217 | 196 | 0.292 |
| TTCT | 2.58E−01 | 1.00E+00 | −0.213 | 197 | 0.552 |
| CAAG | 2.59E−01 | 1.00E+00 | −0.213 | 198 | −0.090 |
| TTGT | 2.63E−01 | 1.00E+00 | 0.211 | 199 | 0.727 |
| TGTG | 2.76E−01 | 1.00E+00 | −0.206 | 200 | −0.329 |
| AACC | 2.78E−01 | 1.00E+00 | −0.205 | 201 | −0.430 |
| ATGC | 2.83E−01 | 1.00E+00 | −0.202 | 202 | 0.432 |
| CCCT | 2.84E−01 | 1.00E+00 | −0.202 | 203 | −0.644 |
| GTAG | 3.06E−01 | 1.00E+00 | −0.194 | 204 | 0.504 |
| CTGT | 3.13E−01 | 1.00E+00 | −0.191 | 205 | 0.213 |
| GAGA | 3.14E−01 | 1.00E+00 | −0.190 | 206 | 0.219 |
| TAAC | 3.17E−01 | 1.00E+00 | 0.189 | 207 | 0.573 |
| TTTG | 3.25E−01 | 1.00E+00 | −0.186 | 208 | 0.597 |
| TTGG | 3.33E−01 | 1.00E+00 | −0.183 | 209 | 0.538 |
| ATCA | 3.34E−01 | 1.00E+00 | 0.183 | 210 | 0.309 |
| CATG | 3.37E−01 | 1.00E+00 | 0.182 | 211 | −0.480 |
| GTGA | 3.45E−01 | 1.00E+00 | 0.179 | 212 | 0.492 |
| TTAG | 3.74E−01 | 1.00E+00 | −0.168 | 213 | 0.574 |
| GCAG | 3.83E−01 | 1.00E+00 | −0.165 | 214 | −0.227 |
| CTTG | 3.84E−01 | 1.00E+00 | −0.165 | 215 | 0.067 |
| CAAC | 4.14E−01 | 1.00E+00 | 0.155 | 216 | 0.035 |
| ACTC | 4.28E−01 | 1.00E+00 | −0.150 | 217 | −0.576 |
| TAGA | 4.46E−01 | 1.00E+00 | 0.145 | 218 | 0.669 |
| TTCA | 4.52E−01 | 1.00E+00 | −0.143 | 219 | 0.602 |
| GGTA | 4.73E−01 | 1.00E+00 | −0.136 | 220 | −0.506 |
| TTCG | 4.83E−01 | 1.00E+00 | −0.133 | 221 | 0.589 |
| GTCT | 5.07E−01 | 1.00E+00 | 0.126 | 222 | −0.021 |
| GCGA | 5.15E−01 | 1.00E+00 | −0.124 | 223 | 0.003 |
| TCTG | 5.22E−01 | 1.00E+00 | −0.122 | 224 | 0.079 |
| TACG | 5.23E−01 | 1.00E+00 | 0.121 | 225 | 0.545 |
| CTTC | 5.30E−01 | 1.00E+00 | −0.119 | 226 | −0.091 |
| CATC | 5.34E−01 | 1.00E+00 | 0.118 | 227 | −0.543 |
| CTGA | 5.47E−01 | 1.00E+00 | 0.114 | 228 | 0.466 |
| GACA | 5.51E−01 | 1.00E+00 | 0.113 | 229 | −0.227 |
| ATAC | 5.94E−01 | 1.00E+00 | 0.101 | 230 | 0.457 |
| CAGA | 6.02E−01 | 1.00E+00 | 0.099 | 231 | 0.418 |
| ATAG | 6.09E−01 | 1.00E+00 | 0.097 | 232 | 0.619 |
| TGCA | 6.34E−01 | 1.00E+00 | 0.091 | 233 | −0.036 |
| TTGC | 6.41E−01 | 1.00E+00 | −0.089 | 234 | 0.620 |
| GGTT | 6.58E−01 | 1.00E+00 | 0.084 | 235 | −0.297 |
| CAGT | 6.64E−01 | 1.00E+00 | −0.083 | 236 | 0.265 |
| GCGT | 6.97E−01 | 1.00E+00 | −0.074 | 237 | −0.063 |
| GCAC | 6.99E−01 | 1.00E+00 | −0.074 | 238 | −0.324 |
| TAGT | 7.13E−01 | 1.00E+00 | 0.070 | 239 | 0.564 |
| TGAC | 7.30E−01 | 1.00E+00 | 0.066 | 240 | 0.484 |
| GTGT | 7.66E−01 | 1.00E+00 | −0.057 | 241 | 0.208 |
| GCCA | 7.68E−01 | 1.00E+00 | −0.056 | 242 | −0.529 |
| ACAG | 7.88E−01 | 1.00E+00 | −0.051 | 243 | −0.464 |
| TGTC | 7.91E−01 | 1.00E+00 | 0.050 | 244 | −0.111 |
| ACCA | 8.13E−01 | 1.00E+00 | −0.045 | 245 | −0.586 |
| ATCT | 8.48E−01 | 1.00E+00 | 0.037 | 246 | −0.032 |
| TAAG | 8.48E−01 | 1.00E+00 | 0.036 | 247 | 0.625 |
| ACAC | 8.59E−01 | 1.00E+00 | −0.034 | 248 | −0.451 |
| TTTC | 8.83E−01 | 1.00E+00 | 0.028 | 249 | 0.611 |
| ACTG | 8.86E−01 | 1.00E+00 | 0.027 | 250 | −0.447 |
| CACA | 9.00E−01 | 1.00E+00 | −0.024 | 251 | −0.499 |
| TTAC | 9.20E−01 | 1.00E+00 | 0.019 | 252 | 0.688 |
| CTAG | 9.28E−01 | 1.00E+00 | −0.017 | 253 | 0.478 |
| GGAA | 9.86E−01 | 1.00E+00 | 0.003 | 254 | −0.104 |
| GATG | 9.91E−01 | 1.00E+00 | 0.002 | 255 | −0.275 |
| CCGT | 9.98E−01 | 1.00E+00 | 0.001 | 256 | −0.231 |

We generally observed that specific end motifs are much better correlated to tissue type analysis (fetal and tumor DNA) compared to total cfDNA.

### D. Ranked End Motifs for Fetal Fraction for 231-600 b P

Table 4 shows a correlation between motif frequency of 4-mer end motifs and fetal fraction from a cohort of pregnant individuals, including first, second and third trimester, with 50% training and testing set (n=60 subjects from Cohort I and II), using DNA sizes from 231-600 bp. Bonferroni's correction was used to adjust the P-value for multiple comparisons. End motifs were ranked according to adjusted P-value from the training set.

| | 5′ End | Pearson r | Adjusted P-value |
|---|---|---|---|
| Rank | Motif | (Testing Set) | (Bonferroni) |
| 1 | GTTC | −0.573 | 9.38E−04 |
| 2 | GTGT | −0.569 | 1.02E−03 |
| 3 | GTTG | −0.538 | 2.16E−03 |
| 4 | GTGC | −0.517 | 3.40E−03 |
| 5 | AATC | −0.495 | 5.39E−03 |
| 6 | CCAA | 0.493 | 5.68E−03 |
| 7 | GTAT | −0.491 | 5.90E−03 |
| 8 | CAAA | 0.490 | 5.95E−03 |
| 9 | CCAG | 0.486 | 6.50E−03 |
| 10 | GTCG | −0.484 | 6.78E−03 |
| 11 | GTAG | −0.482 | 7.05E−03 |
| 12 | CCGA | 0.479 | 7.40E−03 |
| 13 | CAAG | 0.478 | 7.53E−03 |
| 14 | GTCC | −0.478 | 7.59E−03 |
| 15 | ATCC | −0.478 | 7.61E−03 |
| 16 | ATTG | −0.473 | 8.22E−03 |
| 17 | GTCT | −0.473 | 8.24E−03 |
| 18 | GTGA | −0.467 | 9.22E−03 |
| 19 | CCGT | 0.463 | 9.91E−03 |
| 20 | GTGG | −0.461 | 1.03E−02 |
| 21 | ATTC | −0.444 | 1.41E−02 |
| 22 | AATG | −0.440 | 1.50E−02 |
| 23 | CTAA | 0.439 | 1.51E−02 |
| 24 | ATAC | −0.439 | 1.52E−02 |
| 25 | ATGA | −0.430 | 1.76E−02 |
| 26 | GTTT | −0.430 | 1.76E−02 |
| 27 | GGAA | −0.426 | 1.89E−02 |
| 28 | ATGC | −0.423 | 1.98E−02 |
| 29 | AAAC | −0.420 | 2.07E−02 |
| 30 | CCTC | 0.419 | 2.11E−02 |
| 31 | GAAT | −0.413 | 2.31E−02 |
| 32 | GTAA | −0.413 | 2.34E−02 |
| 33 | CAGC | 0.403 | 2.73E−02 |
| 34 | GATC | −0.402 | 2.74E−02 |
| 35 | CCTG | 0.402 | 2.75E−02 |
| 36 | CCGG | 0.399 | 2.88E−02 |
| 37 | CAGA | 0.399 | 2.90E−02 |
| 38 | ATAT | −0.392 | 3.20E−02 |
| 39 | GATT | −0.390 | 3.29E−02 |
| 40 | GATG | −0.386 | 3.49E−02 |
| 41 | TTCC | −0.385 | 3.55E−02 |
| 42 | CAGT | 0.384 | 3.63E−02 |
| 43 | AATT | −0.377 | 4.01E−02 |
| 44 | CTGA | 0.375 | 4.14E−02 |
| 45 | GCGC | −0.372 | 4.27E−02 |
| 46 | ATTA | −0.371 | 4.38E−02 |
| 47 | ATGT | −0.369 | 4.45E−02 |
| 48 | TGCC | 0.368 | 4.55E−02 |
| 49 | CAGG | 0.367 | 4.59E−02 |
| 50 | CCCC | 0.360 | 5.04E−02 |
| 51 | GAAC | −0.360 | 5.09E−02 |
| 52 | ATCT | −0.359 | 5.14E−02 |
| 53 | TGCT | 0.358 | 5.22E−02 |
| 54 | CCCA | 0.356 | 5.32E−02 |
| 55 | GAGG | −0.355 | 5.42E−02 |
| 56 | GAGT | −0.355 | 5.44E−02 |
| 57 | AGGT | −0.354 | 5.52E−02 |
| 58 | GAAG | −0.350 | 5.80E−02 |
| 59 | GCCG | 0.350 | 5.81E−02 |
| 60 | GCCC | 0.350 | 5.82E−02 |
| 61 | ATTT | −0.348 | 5.95E−02 |
| 62 | CTCA | 0.346 | 6.15E−02 |
| 63 | TGCA | 0.345 | 6.20E−02 |
| 64 | CGGC | 0.344 | 6.28E−02 |
| 65 | AGTG | −0.343 | 6.36E−02 |
| 66 | CGTC | 0.343 | 6.36E−02 |
| 67 | ATCG | −0.342 | 6.46E−02 |
| 68 | CTTA | 0.340 | 6.59E−02 |
| 69 | CCGC | 0.337 | 6.88E−02 |
| 70 | ATGG | −0.336 | 6.95E−02 |
| 71 | AACT | −0.336 | 6.96E−02 |
| 72 | CAAC | 0.335 | 7.04E−02 |
| 73 | CATG | 0.331 | 7.44E−02 |
| 74 | CGAC | 0.326 | 7.84E−02 |
| 75 | ACTC | −0.325 | 7.96E−02 |
| 76 | AGTA | −0.325 | 7.96E−02 |
| 77 | GTAC | −0.325 | 8.01E−02 |
| 78 | ATCA | −0.322 | 8.25E−02 |
| 79 | AATA | −0.320 | 8.42E−02 |
| 80 | TGTT | 0.320 | 8.45E−02 |
| 81 | AGAT | −0.316 | 8.90E−02 |
| 82 | AAGT | −0.315 | 9.00E−02 |
| 83 | GGTT | −0.314 | 9.10E−02 |
| 84 | CCAC | 0.308 | 9.80E−02 |
| 85 | AAGG | −0.304 | 1.02E−01 |
| 86 | TTAC | −0.302 | 1.05E−01 |
| 87 | GAGC | −0.301 | 1.05E−01 |
| 88 | AGTC | −0.299 | 1.08E−01 |
| 89 | GAGA | −0.298 | 1.10E−01 |
| 90 | GGGT | −0.298 | 1.10E−01 |
| 91 | TTCT | −0.296 | 1.13E−01 |
| 92 | AACG | −0.295 | 1.13E−01 |
| 93 | GATA | −0.292 | 1.17E−01 |
| 94 | AGTT | −0.287 | 1.24E−01 |
| 95 | CCTA | 0.285 | 1.28E−01 |
| 96 | AAGC | −0.284 | 1.28E−01 |
| 97 | GCCA | 0.276 | 1.40E−01 |
| 98 | AGGC | −0.275 | 1.42E−01 |
| 99 | AAAT | −0.271 | 1.48E−01 |
| 100 | CGCC | 0.269 | 1.51E−01 |
| 101 | CTGT | 0.265 | 1.57E−01 |
| 102 | CGAT | −0.264 | 1.59E−01 |
| 103 | ACGC | −0.263 | 1.60E−01 |
| 104 | CCCT | 0.263 | 1.60E−01 |
| 105 | CCCG | 0.261 | 1.64E−01 |
| 106 | TTTC | −0.259 | 1.67E−01 |
| 107 | CATC | 0.258 | 1.68E−01 |
| 108 | TCCA | −0.256 | 1.72E−01 |
| 109 | TGAC | 0.256 | 1.73E−01 |
| 110 | TTCA | −0.254 | 1.76E−01 |
| 111 | AAAA | 0.254 | 1.76E−01 |
| 112 | ACTT | −0.252 | 1.79E−01 |
| 113 | AACC | −0.252 | 1.79E−01 |
| 114 | TGTC | 0.251 | 1.81E−01 |
| 115 | TGTA | 0.251 | 1.81E−01 |
| 116 | GCTT | −0.246 | 1.90E−01 |
| 117 | CAAT | 0.243 | 1.95E−01 |
| 118 | CGTT | −0.242 | 1.98E−01 |
| 119 | GAAA | −0.241 | 1.99E−01 |
| 120 | TTCG | −0.240 | 2.01E−01 |
| 121 | ATAG | −0.240 | 2.01E−01 |
| 122 | TTGC | −0.239 | 2.03E−01 |
| 123 | ACGG | −0.238 | 2.06E−01 |
| 124 | CTAG | 0.230 | 2.22E−01 |
| 125 | CTAT | 0.220 | 2.43E−01 |
| 126 | TTTG | −0.218 | 2.48E−01 |
| 127 | AGAC | −0.216 | 2.51E−01 |
| 128 | ACTA | −0.216 | 2.52E−01 |
| 129 | GACT | −0.214 | 2.57E−01 |
| 130 | CCTT | 0.211 | 2.64E−01 |
| 131 | TTGA | −0.211 | 2.64E−01 |
| 132 | GGAT | −0.210 | 2.65E−01 |
| 133 | CGAA | −0.209 | 2.67E−01 |
| 134 | GTCA | −0.208 | 2.71E−01 |
| 135 | GGAG | −0.207 | 2.72E−01 |
| 136 | CGGA | 0.207 | 2.73E−01 |
| 137 | AGGA | −0.206 | 2.74E−01 |
| 138 | CTGG | 0.202 | 2.85E−01 |
| 139 | GGCT | −0.200 | 2.89E−01 |
| 140 | TGGA | −0.199 | 2.93E−01 |
| 141 | CGTA | 0.197 | 2.96E−01 |
| 142 | GCGG | −0.197 | 2.96E−01 |
| 143 | GACA | −0.197 | 2.97E−01 |
| 144 | ACCT | −0.196 | 2.98E−01 |
| 145 | CATT | −0.194 | 3.03E−01 |
| 146 | GGGA | −0.193 | 3.06E−01 |
| 147 | TTAT | −0.192 | 3.09E−01 |
| 148 | AAGA | −0.192 | 3.09E−01 |
| 149 | ACTG | −0.191 | 3.12E−01 |
| 150 | CGTG | 0.191 | 3.13E−01 |
| 151 | AAAG | −0.189 | 3.17E−01 |
| 152 | GCTC | −0.187 | 3.23E−01 |
| 153 | TCTA | −0.187 | 3.23E−01 |
| 154 | TCCC | −0.186 | 3.25E−01 |
| 155 | GTTA | −0.185 | 3.27E−01 |
| 156 | ACAT | −0.184 | 3.30E−01 |
| 157 | TGCG | 0.183 | 3.32E−01 |
| 158 | CATA | 0.181 | 3.39E−01 |
| 159 | TTTA | −0.177 | 3.49E−01 |
| 160 | CCAT | 0.175 | 3.55E−01 |
| 161 | CGCA | 0.173 | 3.60E−01 |
| 162 | CTCG | −0.170 | 3.69E−01 |
| 163 | TATG | −0.167 | 3.77E−01 |
| 164 | TTTT | −0.167 | 3.79E−01 |
| 165 | CACA | 0.167 | 3.79E−01 |
| 166 | CGCT | 0.163 | 3.89E−01 |
| 167 | AGCG | −0.163 | 3.90E−01 |
| 168 | CTAC | 0.161 | 3.94E−01 |
| 169 | TTGG | −0.161 | 3.96E−01 |
| 170 | TACC | 0.160 | 3.97E−01 |
| 171 | GCAA | 0.155 | 4.14E−01 |
| 172 | TCAT | −0.154 | 4.16E−01 |
| 173 | TCGA | −0.152 | 4.23E−01 |
| 174 | TTGT | −0.149 | 4.32E−01 |
| 175 | GGAC | −0.147 | 4.38E−01 |
| 176 | CACC | 0.147 | 4.38E−01 |
| 177 | TAGT | −0.140 | 4.60E−01 |
| 178 | TTAA | −0.139 | 4.63E−01 |
| 179 | CTGC | 0.137 | 4.72E−01 |
| 180 | AGCA | −0.132 | 4.87E−01 |
| 181 | AACA | −0.131 | 4.89E−01 |
| 182 | TCTT | −0.131 | 4.91E−01 |
| 183 | AGCT | −0.130 | 4.92E−01 |
| 184 | GGTA | −0.130 | 4.94E−01 |
| 185 | TAAC | −0.127 | 5.05E−01 |
| 186 | CTTC | −0.123 | 5.16E−01 |
| 187 | CACG | 0.123 | 5.17E−01 |
| 188 | TGAT | 0.122 | 5.20E−01 |
| 189 | GGTG | −0.122 | 5.22E−01 |
| 190 | CTTG | 0.121 | 5.24E−01 |
| 191 | TACG | −0.120 | 5.28E−01 |
| 192 | TTAG | −0.119 | 5.32E−01 |
| 193 | ACCG | −0.116 | 5.41E−01 |
| 194 | CGCG | 0.111 | 5.60E−01 |
| 195 | TAAA | 0.109 | 5.65E−01 |
| 196 | TCCT | −0.108 | 5.68E−01 |
| 197 | GCAT | −0.105 | 5.80E−01 |
| 198 | AGCC | −0.101 | 5.97E−01 |
| 199 | ACGA | −0.099 | 6.03E−01 |
| 200 | TCTG | −0.098 | 6.05E−01 |
| 201 | TAGA | −0.090 | 6.36E−01 |
| 202 | GACG | −0.086 | 6.52E−01 |
| 203 | TGTG | 0.086 | 6.52E−01 |
| 204 | CACT | 0.085 | 6.54E−01 |
| 205 | TCAA | −0.083 | 6.64E−01 |
| 206 | AGGG | −0.081 | 6.70E−01 |
| 207 | TAAT | −0.080 | 6.73E−01 |
| 208 | ACGT | 0.080 | 6.73E−01 |
| 209 | TAGC | −0.079 | 6.80E−01 |
| 210 | TCAC | −0.078 | 6.80E−01 |
| 211 | TCAG | −0.076 | 6.90E−01 |
| 212 | GCGT | −0.075 | 6.95E−01 |
| 213 | TCCG | −0.073 | 7.00E−01 |
| 214 | CTCT | −0.071 | 7.10E−01 |
| 215 | TCGG | −0.070 | 7.12E−01 |
| 216 | GCAG | −0.068 | 7.21E−01 |
| 217 | AGAA | −0.068 | 7.23E−01 |
| 218 | GGCA | −0.068 | 7.23E−01 |
| 219 | CTCC | −0.064 | 7.38E−01 |
| 220 | CGAG | 0.061 | 7.50E−01 |
| 221 | TCTC | 0.059 | 7.55E−01 |
| 222 | TCGT | −0.059 | 7.57E−01 |
| 223 | CTTT | 0.059 | 7.58E−01 |
| 224 | GGTC | −0.055 | 7.74E−01 |
| 225 | TAGG | −0.051 | 7.90E−01 |
| 226 | TGGC | −0.050 | 7.92E−01 |
| 227 | ACAC | −0.050 | 7.95E−01 |
| 228 | TGAA | 0.046 | 8.11E−01 |
| 229 | GGGG | −0.041 | 8.28E−01 |
| 230 | TATC | −0.039 | 8.36E−01 |
| 231 | TGGG | 0.032 | 8.65E−01 |
| 232 | TAAG | −0.032 | 8.66E−01 |
| 233 | GGCG | −0.032 | 8.67E−01 |
| 234 | ACAA | 0.029 | 8.77E−01 |
| 235 | ATAA | −0.028 | 8.82E−01 |
| 236 | GCAC | 0.027 | 8.86E−01 |
| 237 | GGGC | −0.027 | 8.87E−01 |
| 238 | GGCC | −0.026 | 8.90E−01 |
| 239 | GCTG | 0.024 | 8.99E−01 |
| 240 | AGAG | −0.023 | 9.02E−01 |
| 241 | GCGA | −0.021 | 9.13E−01 |
| 242 | GCCT | −0.018 | 9.23E−01 |
| 243 | TATA | −0.016 | 9.35E−01 |
| 244 | CGGT | 0.013 | 9.46E−01 |
| 245 | TACA | 0.013 | 9.46E−01 |
| 246 | ACAG | −0.012 | 9.50E−01 |
| 247 | TCGC | 0.011 | 9.54E−01 |
| 248 | TATT | 0.011 | 9.56E−01 |
| 249 | TGGT | 0.010 | 9.59E−01 |
| 250 | CGGG | 0.010 | 9.59E−01 |
| 251 | GCTA | 0.007 | 9.69E−01 |
| 252 | ACCC | −0.007 | 9.71E−01 |
| 253 | TGAG | 0.007 | 9.71E−01 |
| 254 | TACT | 0.005 | 9.77E−01 |
| 255 | GACC | 0.002 | 9.90E−01 |
| 256 | ACCA | 0.002 | 9.92E−01 |

### E. Ranked End Motifs for Tumor Fraction for 231-600 bp

Table 5 shows correlation between motif frequency of 4-mer end motifs and tumor fraction from a cohort of HCC patients, with 50% training and 50% testing set (n=40 subjects from Cohort I and II), using DNA sizes from 231-600 bp. Bonferroni's correction was used to adjust the P-value for multiple comparisons. End motifs were ranked according to adjusted P-value from the training set.

| | | Pearson r | Adjusted P-value |
|---|---|---|---|
| Rank | 5′ End Motif | (Testing Set) | (Bonferroni) |
| 1 | ACGA | 0.790 | 1.39E−20 |
| 2 | TTAT | 0.780 | 2.08E−19 |
| 3 | CAGT | 0.778 | 3.20E−19 |
| 4 | GTGG | 0.771 | 9.82E−19 |
| 5 | ATGG | 0.771 | 1.03E−18 |
| 6 | ACGG | 0.728 | 6.51E−16 |
| 7 | CAGG | 0.715 | 3.35E−15 |
| 8 | AGGT | 0.715 | 3.66E−15 |
| 9 | ACGT | 0.707 | 9.59E−15 |
| 10 | CAGA | 0.694 | 4.47E−14 |
| 11 | GTAG | 0.685 | 1.34E−13 |
| 12 | AGGC | 0.684 | 1.51E−13 |
| 13 | ATGC | 0.674 | 4.55E−13 |
| 14 | ATAG | 0.666 | 1.09E−12 |
| 15 | GTGC | 0.663 | 1.42E−12 |
| 16 | AGGA | 0.659 | 2.18E−12 |
| 17 | AAAA | 0.653 | 4.18E−12 |
| 18 | GTGA | 0.652 | 4.35E−12 |
| 19 | AGGG | 0.639 | 1.53E−11 |
| 20 | TTAG | 0.638 | 1.71E−11 |
| 21 | CCTT | −0.634 | 2.50E−11 |
| 22 | GTGT | 0.628 | 4.51E−11 |
| 23 | CCTG | −0.627 | 4.85E−11 |
| 24 | TTAA | 0.617 | 1.19E−10 |
| 25 | CCTC | −0.615 | 1.45E−10 |
| 26 | AAGT | 0.615 | 1.50E−10 |
| 27 | ATGA | 0.611 | 2.01E−10 |
| 28 | AAGA | 0.609 | 2.36E−10 |
| 29 | ATGT | 0.602 | 4.41E−10 |
| 30 | CCCT | −0.598 | 6.10E−10 |
| 31 | AAGC | 0.590 | 1.18E−09 |
| 32 | ACGC | 0.582 | 2.20E−09 |
| 33 | CACT | −0.570 | 5.45E−09 |
| 34 | ATAC | 0.568 | 6.61E−09 |
| 35 | TTGG | 0.564 | 8.48E−09 |
| 36 | AAGG | 0.559 | 1.28E−08 |
| 37 | AGAA | 0.557 | 1.43E−08 |
| 38 | CCCC | −0.554 | 1.73E−08 |
| 39 | GAGA | 0.550 | 2.35E−08 |
| 40 | GCGT | 0.547 | 2.89E−08 |
| 41 | CCCA | −0.546 | 3.22E−08 |
| 42 | ATAA | 0.539 | 5.17E−08 |
| 43 | CGCT | −0.535 | 6.43E−08 |
| 44 | CCAC | −0.531 | 8.50E−08 |
| 45 | CTGG | 0.531 | 8.77E−08 |
| 46 | CGTC | −0.530 | 9.20E−08 |
| 47 | GTAA | 0.528 | 1.09E−07 |
| 48 | CCAG | −0.525 | 1.28E−07 |
| 49 | TGGA | 0.525 | 1.32E−07 |
| 50 | TAGG | 0.516 | 2.35E−07 |
| 51 | TTAC | 0.514 | 2.64E−07 |
| 52 | TAGA | 0.512 | 2.84E−07 |
| 53 | CCCG | −0.511 | 3.17E−07 |
| 54 | CAGC | 0.510 | 3.27E−07 |
| 55 | TGGT | 0.508 | 3.74E−07 |
| 56 | CCAA | −0.508 | 3.81E−07 |
| 57 | TTCT | 0.506 | 4.30E−07 |
| 58 | CACC | −0.504 | 4.66E−07 |
| 59 | AGAC | 0.504 | 4.69E−07 |
| 60 | CGCA | −0.503 | 5.00E−07 |
| 61 | TTCA | 0.503 | 5.21E−07 |
| 62 | CGAC | −0.500 | 6.12E−07 |
| 63 | AGAG | 0.500 | 6.13E−07 |
| 64 | AAAG | 0.498 | 6.90E−07 |
| 65 | GGGA | 0.498 | 7.04E−07 |
| 66 | CTAG | 0.498 | 7.05E−07 |
| 67 | GTAC | 0.495 | 7.99E−07 |
| 68 | CGCC | −0.489 | 1.14E−06 |
| 69 | TTGT | 0.486 | 1.41E−06 |
| 70 | CCAT | −0.479 | 2.03E−06 |
| 71 | GAGG | 0.477 | 2.30E−06 |
| 72 | GAAA | 0.471 | 3.15E−06 |
| 73 | CGTT | −0.470 | 3.31E−06 |
| 74 | GTCA | 0.467 | 3.90E−06 |
| 75 | ATAT | 0.465 | 4.32E−06 |
| 76 | ATCA | 0.462 | 5.31E−06 |
| 77 | CGTG | −0.459 | 6.10E−06 |
| 78 | CCTA | −0.449 | 1.02E−05 |
| 79 | CGCG | −0.447 | 1.12E−05 |
| 80 | CGAG | −0.444 | 1.31E−05 |
| 81 | TTTT | 0.443 | 1.35E−05 |
| 82 | TCGG | 0.441 | 1.50E−05 |
| 83 | CTGT | 0.438 | 1.80E−05 |
| 84 | CGAT | −0.438 | 1.80E−05 |
| 85 | TAAT | 0.434 | 2.12E−05 |
| 86 | TGGG | 0.424 | 3.52E−05 |
| 87 | GGGG | 0.423 | 3.64E−05 |
| 88 | CTAT | 0.421 | 3.97E−05 |
| 89 | TCGT | 0.415 | 5.27E−05 |
| 90 | GCGA | 0.409 | 7.00E−05 |
| 91 | GAGT | 0.408 | 7.10E−05 |
| 92 | ACCC | −0.407 | 7.66E−05 |
| 93 | GGGT | 0.399 | 1.09E−04 |
| 94 | AAAT | 0.398 | 1.14E−04 |
| 95 | GACC | −0.390 | 1.60E−04 |
| 96 | CACG | −0.389 | 1.62E−04 |
| 97 | TGCT | −0.389 | 1.68E−04 |
| 98 | TGCC | −0.388 | 1.70E−04 |
| 99 | TTGC | 0.388 | 1.75E−04 |
| 100 | GAAC | 0.386 | 1.84E−04 |
| 101 | GGCA | 0.386 | 1.90E−04 |
| 102 | GAAG | 0.378 | 2.64E−04 |
| 103 | TAGT | 0.375 | 2.94E−04 |
| 104 | TTTG | 0.369 | 3.79E−04 |
| 105 | TTCC | 0.366 | 4.13E−04 |
| 106 | GTTG | 0.366 | 4.27E−04 |
| 107 | CGAA | −0.364 | 4.50E−04 |
| 108 | TGCA | −0.360 | 5.26E−04 |
| 109 | GTAT | 0.359 | 5.42E−04 |
| 110 | GGAC | 0.357 | 5.89E−04 |
| 111 | AGCA | 0.355 | 6.45E−04 |
| 112 | CATC | −0.353 | 6.84E−04 |
| 113 | TCTC | −0.349 | 7.90E−04 |
| 114 | TGTC | −0.349 | 8.11E−04 |
| 115 | ACCT | −0.347 | 8.79E−04 |
| 116 | CACA | −0.346 | 8.92E−04 |
| 117 | ATCG | 0.343 | 9.87E−04 |
| 118 | ATTG | 0.342 | 1.03E−03 |
| 119 | TAGC | 0.339 | 1.15E−03 |
| 120 | TCTA | −0.337 | 1.22E−03 |
| 121 | TCTT | −0.334 | 1.36E−03 |
| 122 | GCGG | 0.334 | 1.39E−03 |
| 123 | GGAA | 0.332 | 1.47E−03 |
| 124 | GAAT | 0.332 | 1.47E−03 |
| 125 | GAGC | 0.330 | 1.61E−03 |
| 126 | CCGT | 0.329 | 1.65E−03 |
| 127 | TCGA | 0.328 | 1.68E−03 |
| 128 | AAAC | 0.324 | 1.92E−03 |
| 129 | GGGC | 0.320 | 2.23E−03 |
| 130 | TGTT | −0.319 | 2.28E−03 |
| 131 | TCCC | −0.318 | 2.39E−03 |
| 132 | TGCG | −0.317 | 2.50E−03 |
| 133 | TCCT | −0.315 | 2.61E−03 |
| 134 | AGAT | 0.313 | 2.87E−03 |
| 135 | GCCT | −0.302 | 3.96E−03 |
| 136 | CAAC | −0.302 | 4.05E−03 |
| 137 | TGTA | −0.301 | 4.10E−03 |
| 138 | TACC | −0.301 | 4.14E−03 |
| 139 | GCCC | −0.300 | 4.33E−03 |
| 140 | CGGC | −0.295 | 4.93E−03 |
| 141 | GCTC | −0.295 | 4.96E−03 |
| 142 | ACTC | −0.295 | 5.00E−03 |
| 143 | GGTG | 0.292 | 5.53E−03 |
| 144 | TGGC | 0.289 | 6.02E−03 |
| 145 | ATCC | 0.281 | 7.67E−03 |
| 146 | TGAT | 0.281 | 7.73E−03 |
| 147 | AGTG | 0.279 | 8.07E−03 |
| 148 | ACCG | −0.278 | 8.43E−03 |
| 149 | GGAG | 0.277 | 8.60E−03 |
| 150 | GCTT | −0.274 | 9.33E−03 |
| 151 | CTAC | 0.274 | 9.46E−03 |
| 152 | TTCG | 0.273 | 9.55E−03 |
| 153 | TCAC | −0.273 | 9.68E−03 |
| 154 | CTAA | 0.268 | 1.10E−02 |
| 155 | TCCA | −0.260 | 1.40E−02 |
| 156 | ACTT | −0.259 | 1.41E−02 |
| 157 | TTGA | 0.254 | 1.63E−02 |
| 158 | GGTA | 0.254 | 1.65E−02 |
| 159 | AGCG | 0.253 | 1.68E−02 |
| 160 | CTTC | −0.242 | 2.21E−02 |
| 161 | TGTG | −0.239 | 2.43E−02 |
| 162 | CTCA | 0.238 | 2.48E−02 |
| 163 | ATTA | 0.238 | 2.48E−02 |
| 164 | ACTG | −0.229 | 3.06E−02 |
| 165 | CTTT | −0.226 | 3.30E−02 |
| 166 | GCGC | 0.224 | 3.49E−02 |
| 167 | GACG | −0.221 | 3.78E−02 |
| 168 | CATT | −0.218 | 3.97E−02 |
| 169 | GGAT | 0.218 | 3.99E−02 |
| 170 | TCGC | 0.218 | 4.05E−02 |
| 171 | GGCC | −0.211 | 4.75E−02 |
| 172 | GCCG | −0.205 | 5.37E−02 |
| 173 | TGAC | −0.204 | 5.54E−02 |
| 174 | AATG | 0.203 | 5.62E−02 |
| 175 | GTCG | 0.202 | 5.80E−02 |
| 176 | TACT | −0.200 | 5.97E−02 |
| 177 | GACT | −0.199 | 6.20E−02 |
| 178 | GCTG | −0.195 | 6.64E−02 |
| 179 | TATT | 0.191 | 7.35E−02 |
| 180 | CAAG | −0.189 | 7.58E−02 |
| 181 | GATG | 0.188 | 7.75E−02 |
| 182 | CTGC | 0.185 | 8.32E−02 |
| 183 | GTTA | 0.184 | 8.38E−02 |
| 184 | AACA | 0.183 | 8.58E−02 |
| 185 | AGTA | 0.183 | 8.59E−02 |
| 186 | CCGC | −0.180 | 9.13E−02 |
| 187 | AATA | 0.179 | 9.36E−02 |
| 188 | CCGG | 0.178 | 9.52E−02 |
| 189 | TATA | 0.177 | 9.75E−02 |
| 190 | TCTG | −0.176 | 9.91E−02 |
| 191 | GATA | 0.176 | 9.91E−02 |
| 192 | TTTA | 0.171 | 1.09E−01 |
| 193 | TTTC | 0.167 | 1.18E−01 |
| 194 | CAAA | −0.163 | 1.27E−01 |
| 195 | AGTC | 0.159 | 1.36E−01 |
| 196 | GGCG | 0.157 | 1.41E−01 |
| 197 | GCCA | −0.156 | 1.45E−01 |
| 198 | GCTA | −0.155 | 1.47E−01 |
| 199 | AATT | 0.151 | 1.57E−01 |
| 200 | TACA | −0.151 | 1.58E−01 |
| 201 | CGGT | 0.145 | 1.76E−01 |
| 202 | GTCC | 0.144 | 1.79E−01 |
| 203 | TAAC | −0.143 | 1.81E−01 |
| 204 | GACA | 0.142 | 1.85E−01 |
| 205 | TAAG | 0.128 | 2.31E−01 |
| 206 | ACAT | −0.128 | 2.32E−01 |
| 207 | TCAA | −0.128 | 2.32E−01 |
| 208 | AGCC | 0.125 | 2.43E−01 |
| 209 | TCCG | −0.121 | 2.58E−01 |
| 210 | ATTT | 0.121 | 2.59E−01 |
| 211 | TATG | 0.118 | 2.72E−01 |
| 212 | TCAT | −0.116 | 2.80E−01 |
| 213 | TACG | −0.111 | 2.98E−01 |
| 214 | AATC | 0.111 | 3.01E−01 |
| 215 | AACG | 0.109 | 3.08E−01 |
| 216 | TAAA | 0.108 | 3.15E−01 |
| 217 | CGGA | −0.083 | 4.40E−01 |
| 218 | GCAC | −0.083 | 4.40E−01 |
| 219 | ACTA | −0.082 | 4.44E−01 |
| 220 | ATCT | 0.079 | 4.61E−01 |
| 221 | TGAA | 0.077 | 4.75E−01 |
| 222 | GGTC | 0.075 | 4.85E−01 |
| 223 | ATTC | 0.074 | 4.88E−01 |
| 224 | GCAA | −0.070 | 5.13E−01 |
| 225 | CGTA | −0.068 | 5.26E−01 |
| 226 | GTCT | 0.066 | 5.41E−01 |
| 227 | CTCG | −0.065 | 5.45E−01 |
| 228 | CGGG | 0.064 | 5.52E−01 |
| 229 | GGCT | −0.062 | 5.64E−01 |
| 230 | ACCA | −0.062 | 5.65E−01 |
| 231 | TCAG | −0.061 | 5.73E−01 |
| 232 | GATT | −0.059 | 5.84E−01 |
| 233 | CTTG | 0.057 | 5.98E−01 |
| 234 | GCAT | −0.052 | 6.27E−01 |
| 235 | TATC | −0.050 | 6.44E−01 |
| 236 | GTTC | −0.049 | 6.46E−01 |
| 237 | GATC | −0.049 | 6.50E−01 |
| 238 | AGTT | 0.048 | 6.52E−01 |
| 239 | GCAG | −0.043 | 6.91E−01 |
| 240 | AACC | −0.034 | 7.53E−01 |
| 241 | CTTA | −0.033 | 7.56E−01 |
| 242 | CTCC | −0.033 | 7.58E−01 |
| 243 | AACT | 0.032 | 7.69E−01 |
| 244 | CATG | −0.029 | 7.86E−01 |
| 245 | CTGA | 0.025 | 8.16E−01 |
| 246 | ACAA | 0.024 | 8.22E−01 |
| 247 | CTCT | 0.023 | 8.34E−01 |
| 248 | CAAT | 0.022 | 8.40E−01 |
| 249 | AGCT | 0.020 | 8.55E−01 |
| 250 | ACAC | −0.019 | 8.56E−01 |
| 251 | ACAG | −0.016 | 8.84E−01 |
| 252 | CCGA | 0.014 | 8.96E−01 |
| 253 | CATA | 0.013 | 9.03E−01 |
| 254 | GGTT | −0.013 | 9.04E−01 |
| 255 | GTTT | −0.003 | 9.81E−01 |
| 256 | TGAG | −0.001 | 9.95E−01 |

1. A method measuring a first concentration of all cell-free DNA in a biological sample of a subject, the method comprising:
measuring a first amount of a plurality of cell-free DNA fragments having a first size in the biological sample, wherein (1) the first size has an upper bound less than 231 bp and a lower bound less than 161 bp or (2) the first size has a lower bound greater than 160 bp and an upper bound greater than 230 bp; and
determining the first concentration of all cell-free DNA in the biological sample using the first amount and one or more calibration amounts of cell-free DNA fragments having the first size determined from one or more calibration samples, each having a known concentration of cell-free DNA.

2. The method of claim 1, further comprising:
measuring a second amount of the plurality of cell-free DNA fragments in the biological sample having a set of one or more sequence motifs corresponding to ending sequences of the plurality of cell-free DNA fragments, wherein determining the first concentration of all cell-free DNA in the biological sample further uses the second amount and one or more additional calibration amounts determined from one or more additional calibration samples, each having a known concentration of cell-free DNA.

3. The method of claim 1, wherein the one or more calibration amounts are a plurality of calibration amounts and the one or more calibration samples are a plurality of calibration samples, the method further comprising:
measuring other amounts of cell-free DNA fragments having other sizes, wherein determining the first concentration of all cell-free DNA in the biological sample includes using a machine learning model that (a) operates on the first amount and the other amounts and (b) is trained using a training set that include the plurality of calibration amounts and known concentrations of the plurality of calibration samples.

4. The method of claim 3, wherein the other sizes are in a range of 21 bp to 600 bp.

5. The method of claim 4, wherein the first size and the other sizes include all sizes within a range of 21 bp to 600 bp.

6. The method of claim 4, wherein the first size and the other sizes include all sizes within a range 21 bp to 160 bp and 231 bp to 600 bp.

7. The method claim 3, wherein the first size and the other sizes include a plurality of size ranges, each of a specified width, and wherein the first amount and the other amounts are each determine for one of the plurality of size ranges.

8. The method of claim 1, wherein the first size has the upper bound less than 231 bp and the lower bound less than 161 bp.

9. The method of claim 1, wherein the first size has the lower bound greater than 160 bp and the upper bound greater than 230 bp.

10. The method of claim 1, wherein the first size is a size range.

11. The method of claim 1, wherein the first amount is normalized by a total number of the plurality of cell-free DNA fragments.

12. A method measuring a first concentration of all cell-free DNA in a biological sample of a subject, the method comprising:
measuring a first amount of a plurality of cell-free DNA fragments in the biological sample having a set of one or more sequence motifs corresponding to ending sequences of the plurality of cell-free DNA fragments; and
determining the first concentration of all cell-free DNA in the biological sample using the first amount and one or more calibration amounts of cell-free DNA fragments having the set of one or more sequence motifs determined from one or more calibration samples, each having a known concentration of cell-free DNA.

13. The method of claim 12, wherein the first amount is measured using a probe-based technique.

14. The method of claim 12, further comprising:
measuring a second amount of the plurality of cell-free DNA fragments having a first size in the biological sample, wherein (1) the first size has an upper bound less than 231 bp and a lower bound less than 161 bp or (2) the first size has a lower bound greater than 160 bp and an upper bound greater than 230 bp, wherein determining the first concentration of all cell-free DNA in the biological sample further uses the second amount and one or more additional calibration amounts determined from one or more additional calibration samples, each having a known concentration of cell-free DNA.

15. The method of claim 2, wherein the one or more calibration samples are the one or more additional calibration samples.

16. The method of claim 12, wherein the set of one or more sequence motifs is a set of sequence motifs.

17. The method of claim 16, further comprising:
storing a set of reference F-profiles, wherein each reference F-profile of the set of reference F-profiles:
identifies, for each sequence motif of the set of sequence motifs, a proportion of cell-free DNA fragments having the sequence motif, and
is associated with a type of fragmentation factors;
determining a sample end-motif profile by measuring other amounts of cell-free DNA fragments having other sequence motifs corresponding to ending sequences of the plurality of cell-free DNA fragments, the sample end-motif profile including the first amount and the other amounts; and
determining proportional contributions of the set of reference F-profiles whose proportional aggregation provide the sample end-motif profile, wherein the proportional contributions sum to one,
wherein determining the first concentration of all cell-free DNA in the biological sample uses a first proportional contribution of a first reference F-profile of the set of reference F-profiles and one or more reference contributions determined from the one or more calibration samples.

18. The method of claim 17, wherein the plurality of cell-free DNA fragments has a first size with a lower bound greater than 160 bp and an upper bound greater than 230 bp.

19. The method of claim 17, wherein determining the first concentration of all cell-free DNA in the biological sample comprises comparing the first proportional contribution to the one or more reference contributions.

20. The method of claim 19, wherein the one or more reference contributions are a plurality of reference contributions, and wherein comparing the first proportional contribution to the one or more reference contributions uses a calibration function determined using the plurality of reference contributions and the known concentrations.

21.-65. (canceled)


SEQUENCE LISTING (2 sequences)

SEQ ID NO: 1 - DNA, 21 bp, synthetic construct
  source 1..21: mol_type = other DNA
gggaggtctgtggaccctatc

SEQ ID NO: 2 - DNA, 16 bp, synthetic construct
  source 1..16: mol_type = other DNA
cttccccaaccatcag